(19) United States
(12) Patent Application Publication
(10) Pub. No.: US 2006/0008800 A1
(43) Pub. Date: Jan. 12, 2006

(54) Unc-13 in the modulation of neurotransmission and secretion events

(76) Inventors: Christian Rosenmund, Houston, TX (US); Nils Brose, Gottingen (DE); Jeong-Seon Rhee, Houston, TX (US); Andrea Betz, Gottingen (DE); Jens Rettig, Homburg (DE); Uri Ashery, Tel Aviv (IL); Harald Junge, Burlingame, CA (US)

Correspondence Address: PILLSBURY WINTHROP SHAW PITTMAN LLP, ATTENTION: DOCKETING DEPARTMENT, P.O BOX 10500, McLean, VA, 22102, US

(21) Appl. No.: 10/495,083

(22) PCT Filed: Oct. 29, 2002
(86) PCT No.: PCT/EP02/12072

§ 371 Date: Oct. 28, 2004

(51) Int. Cl. C12Q 1/68; C07H 21/04; C12N 9/02

(52) U.S. Cl. 435/6; 435/69.1; 435/189; 435/320.1; 435/325; 536/23.2

(57) ABSTRACT

The present invention relates to a method for identifying and/or obtaining a molecule which is capable of modifying secretion processes comprising the steps of a) contacting an unc-13 molecule or (a) part(s) or (a) fragment(s) thereof with a candidate molecule; b) measuring and/or detecting a response; and c) comparing said response to a standard response as measured in the absence of said candidate molecule. Furthermore, a method for identifying and/or obtaining a isoform-specific modulator of isoform-specific Munc13 activity is disclosed. The invention also provides for pharmaceutical and diagnostic compositions as well as for uses of antagonist or agonists as identified and/or obtained by the inventive method for the preparation of pharmaceutical and diagnostic compositions.

The present invention relates to a method for identifying and/or obtaining a molecule which is capable of modifying secretion processes comprising the steps of a) contacting an unc-13 molecule or (a) part(s) or (a) fragment(s) thereof with a candidate molecule; b) measuring and/or detecting a response; and c) comparing said response to a standard response as measured in the absence of said candidate molecule. Furthermore, a method for identifying and/or obtaining a isoform-specific modulator of isoform-specific Munc13 activity is disclosed. The invention also provides for pharmaceutical and diagnostic compositions as well as for uses of antagonist or agonists as identified and/or obtained by the inventive method for the preparation of pharmaceutical and diagnostic compositions.

Several documents are cited throughout the text of this specification. The disclosure content of each of the documents cited herein (including any manufacturer's specifications, instructions, etc.) are hereby incorporated by reference.

Neurotransmitter release from neurons is restricted to synaptic active zones. At these electron dense presynaptic plasma membrane specializations, the final steps of synaptic vesicle exocytosis take place in a highly coordinated manner. Typically, only a fraction of vesicles in close proximity of the active zone plasma membrane are primed, i.e. fusion competent, and able to exocytose their content in response to an arriving action potential and concomitant Ca²⁺ influx. The size of this readily releasable vesicle pool and its dynamic regulation by the priming machinery determine the efficacy and signaling capacity of synapses (Südhof, 1995; Zucker, 1996).

Transient and long lasting changes in the efficacy of synaptic transmission between neurons mediate adaptive properties of the brain and contribute to learning and memory processes. Alterations of synaptic strength, which are best characterized in synaptic long term potentiation, occur in diverse brain regions where they involve successive and spatially segregated modulations of pre- and postsynaptic processes, including neurotransmitter release, transmitter receptor activation, gene expression, protein synthesis, and synaptic structure (Malenka, 1999; Soderling, 2000).

Presynaptic short term plasticity allows a given synapse to rapidly alter its transmitter release characteristics in response to acute changes in activation patterns. In most synapses, the transduction of action potentials into synaptic transmitter release is relatively inefficient (Ma, 1999; Malgaroli, 1999; Rosenmund, 1993).

In almost all studies examining presynaptic short term plasticity, alterations of presynaptic efficacy were shown to be associated with the buildup of intraterminal Ca²⁺ during high frequency stimulation (Zucker, 1999). However, the mechanism by which Ca²⁺ modulates short term changes in synaptic efficacy has remained enigmatic. In principle, interference of Ca²⁺ with every step of the synaptic vesicle cycle could result in short term changes of synaptic release probability. Thus, short term plasticity may be caused by a direct effect of Ca²⁺ on proteins regulating vesicle translocation, tethering, docking, priming, fusion or even endocytosis. In addition, rises in intraterminal Ca²⁺ concentrations could regulate cytoskeletal proteins or kinases/phosphatases, which, in turn, modulate components of the synaptic release machinery.

Synapses in the central nervous system display a striking heterogeneity in presynaptic properties including morphology, synaptic or vesicular release probability, and short term plasticity (Harris, 1989; Hessler, 1993; Huang, 1997; Rosenmund, 1993; Schikorski, 1997; Walmsley, 1998). Interestingly, such heterogeneity is not only observed among synapses from different types of neurons but also detectable between synapses formed by a single axon of an individual nerve cell (Murthy, 1997; Reyes, 1998; Rosenmund, 1993; Thomson, 1997). In none of these cases, the molecular mechanisms underlying synapse heterogeneity are known, and their physiological relevance is unclear. This is mostly due to the fact that it is very difficult to study presynaptic characteristics of individual synapses or to experimentally separate pools of synapses formed by a given individual axon.

Presynaptically, changes in the shape of action potentials, the size and duration of Ca²⁺ transients, or the refilling kinetics and size of readily releasable vesicle pools can contribute to transient and long term increases in evoked synaptic transmitter release (Hawkins, 1993; Nayak, 1999; Bennett, 2000). In addition to physiological stimuli such as high frequency action potential trains, increases in presynaptic efficacy in many experimental paradigms are readily induced by β phorbol esters (β-PEs; Majewski, 1998). These diterpenes are functional analogues of the endogenous second messenger diacylglycerol and bind with high affinity to the zinc finger-like C₁ domains of a diverse group of mammalian diacylglycerol/β-PE receptor proteins, including PKCs α, βI, βII, γ, δ, ε, η, θ, μ, and ν, Munc13-1, -2, and -3, α1/2 and β1/2 chimaerins, and RasGRP (Kazanietz, 2000).

The mechanism by which diacylglycerol and β-PEs regulate presynaptic function is very important because modulation of transmitter release by G-protein coupled receptors which in turn stimulate diacylglycerol production represents a regulatory second messenger pathway that controls synaptic transmission in brain and further secretion events in other organs. A large body of indirect pharmacological evidence suggests that β-PEs (and thus diacylglycerol) enhance transmitter release by activating PKCs. Indeed, the strong stimulation of presynaptic function by β-PEs is partially inhibited by various more or less specific antagonists of PKCs and was therefore in the past almost exclusively attributed to an activating effect of the drugs on PKC function (Majewski, 1998). K⁺ channels, Ca²⁺ channels or components of the transmitter secretion apparatus such as the SNARE protein SNAP-25 and the presynaptic regulatory factor GAP43 have been suggested as possible targets of β-PEs/PKCs in the modulation of presynaptic function (Parfitt, 1993; de Graan, 1994; Shimazaki, 1996; Redman, 1997; Hoffman, 1998; Minami, 1998; Stevens, 1998; Hori, 1999; Yawo, 1999a and 1999b; Honda, 2000; Oleskevich, 2000; Oleskevich, 2000; Waters, 2000).

Published studies on the role of PKCs in the regulation or presynaptic function are entirely based on pharmacological manipulations using β-PEs and PKC inhibitors. Nevertheless, the concept that PKCs are important physiological mediators of enhanced neurotransmitter output with a role in transient and long term potentiation of synaptic strength has become generally accepted because the effects of β-PEs and commonly used PKC inhibitors are so robust. However, β-PEs and many other pharmacological tools used to modify PKC function interact equally well with other β-PE receptors (Betz, 1998), and a number of other drugs are only moderately specific for PKCs. Several recent studies suggest the presence of alternative β-PE receptors with a regulatory role in neurotransmitter release (Stevens, 1998; Honda, 2000; Hori, 1999; Redman, 1997; Iwasaki, 2000; Waters, 2000).

As discussed herein above, the transduction of (nervous) signals depends on an efficient provision of fusion-competent vesicles (priming) as well as effective modification of signal transduction pathways. Similar events are supposed to be involved in other secretory processes, like hormone-releases (e.g. release of insulin or glucagon). Yet, a plurality of diseases and disorders are known in the art, where a correct vesicle generation, vesicle fusion and/or the release of neurotransmitters or other transmitters or hormones is disturbed. Such disease, disorders comprise, but are not limited to schizophrenia, epilepsy, Parkinson's disease, diabetes mellitus, hyper- and hypothyriodism, Morbus Addison, Morbus Cushing or hypertonus.

Therefore, there is a need in the art to provide for means and methods to influence specifically biological secretion processes without damaging effects, like for example described in the stimulation of PKCs (Protein kinase C) by phorbol esters and the like. In this respect, the technical problem of the present invention is to provide for tools which allow for the modulation of metabolic conditions influencing biological secretion processes, in particular neurotransmitter and/or hormone release.

The solution to said technical problem is achieved by the embodiments characterized in the claims.

Accordingly, the present invention relates to a method for identifying and/or obtaining a molecule which is capable of modifying secretion processes comprising the steps of a) contacting an unc-13 molecule or (a) part(s) or (a) fragment(s) thereof with a candidate molecule; b) measuring and/or detecting a response; and c) comparing said response to a standard response as measured in the absence of said candidate molecule.

The term “capable of modifying secretion processes” relates in context of this invention to the modification of the production/generation of secretory vesicles, in particular of vesicles for transmitter and/or hormone release and most preferably of neurotransmitter release. Furthermore, said term relates in particular to the capability modify the fusion competence of transmitter-or hormone vesicles, preferably of vesicles involved in neurotransmitter release on synapses. The term “modification of secretion processes” also relates to modification of vesicle priming, i.e. the modification of the prerequisite fusion competence before fusion events of said secretory vesicles, preferably of neurotransmitter or hormone vesicles, can take place, for example upon a trigger related to an increase in intracellular Ca²⁺ concentration. The term “modification” as employed herein relates to activation as well as to inhibition processes, i.e said modification comprises up- or down-regulations of said secretory events. “Modification” also relates to activity-dependent regulation (i.e. partial or complete activation or inhibition) of priming events, as, inter alia, adaptational processes that maintain synaptic transmission or hormone secretion at high action potential frequencies, the stimulation of the vegetative nervous system or stress reactions.

The term “unc13 molecule” relates to a molecule of the subgroup of the unc-family (Hosono, 1987, J. Neurochem. 49, 1820-1823; Hosono, Neurosci. Lett, 128, 243-244) which was first described in C. elegans. Brose et al (1995) described the mammalian homologues of unc 13 (Munc13). The term “unc13” in context of this invention relates not only to the mammalian Munc 13s, but also to unc13 molecules of lower vertebrates or non-vertebrates, like unc-13 from Xenopus, unc13 of C. elegans or Drosphila unc13 (Dunc13). Yet, as will be discussed herein below, most preferably the term “unc13” relates to the mammalian homologues “Munc-13”, and most preferably to the specific isoforms of Munc13 molecules (Munc13s). The mammalian Munc-13 molecules constitute a family of three members, i.e., Munc13-1, Munc13-2 and Munc 13-3 (Brose, 1995; Augustin, 1999a) The term “unc13” or “Munc13” does not only relate to brain-specific unc13 or Munc13 homologues but also comprises ubiquitously expressed unc13/Munc13 molecules (Asheri, 2000; Betz 2001)

Most preferably, the present invention relates to a method for for identifying and/or obtaining a unc13-isoform specific modulator which is able of modifying the activity of specific isoforms of unc-13, and in particular of Munc13s. Said activity of unc13-, preferably of Munc13-isoforms comprises, but is not limited to the above described modification of secretion processes.

Whereas the prior art has postulated that all known unc13/Munc13 isoforms have similar if not identical physiological properties, here it was surprisingly found that unc13 isofoms, and in particular the Munc 13 isofoms Munc13-1, Munc13-2 and Munc13-3 show a completely different and unique physiological behaviour and that said isoforms are physiologically highly divers. In particular, and as documented in the appended examples, Munc13-1 shows an ubiquitous expression but moderate depression of synaptic responses during high action potential frequencies. In contrast, Munc13-2, which is expressed in only a subset of neurons and in a subset of synapses formed by a single axon shows dramatically increased synaptic responses to such high action potential frequencies. Furthermore, it was found that Munc13-3 has similar physiological properties as Munc13-2, but plays a role in synaptic transmission in other areas of the brain. In addition the present invention surprisingly shows that Munc13 molecules and not PKCs are the main presynaptic beta-phorbolester and diacylglycerol receptors in neurons. Accordingly, the present invention provides for means and methods for identifying and/or obtaining unc13/Munc13-isoform specific modulators of unc13/Munc13 activity or expression whereby, preferably, said modulator does not interfere and/or interact with PKCs.

Therefore, the present invention provides for a method for identifying and/or obtaining a isoform-specific modulator of Munc-13-1 activity comprising the steps of: a) contacting a Munc-13-1 molecule or (a) part(s) or (a) fragment(s) thereof with a candidate molecule; b) measuring and/or detecting a response; and c) comparing said response to a standard response as measured in the absence of said candidate molecule; d) contacting the identified and/or obtained candidate molecule of step c) with Munc13-2 or (a) part(s) or (a) fragment(s) thereof or Munc13-3 or (a) part(s) or (a) fragment(s) thereof; e) measuring and/or detecting whether said candidate molecule interacts with said Munc13-2 or (a) part(s) or (a) fragment(s) thereof or said Munc13-3 or (a) part(s) or (a) fragment(s) thereof; and f) selecting a candidate molecule which is not capable of interacting with Munc13-2 or (a) part(s) or (a) fragment(s) thereof or with Munc13-3 or (a) part(s) or (a) fragment(s).

Similarly, the present invention provides for a method for identifying and/or obtaining a isoform-specific modulator of Munc-13-2 activity comprising the steps of: a) contacting a Munc-13-2 molecule or (a) part(s) or (a) fragment(s) thereof with a candidate molecule; b) measuring and/or detecting a response; and c) comparing said response to a standard response as measured in the absence of said candidate molecule; d) contacting the identified and/or obtained candidate molecule of step c) with Munc13-1 or (a) part(s) or (a) fragment(s) or Munc13-3 or (a) part(s) or (a) fragment(s) thereof; e) measuring and/or detecting whether said candidate molecule interacts with said Munc13-1 or (a) part(s) or (a) fragment(s) thereof or said Munc13-3 or (a) part(s) or (a) fragment(s) thereof; and f) selecting a candidate molecule which is not capable of interacting with Munc13-1 or (a) part(s) or (a) fragment(s) thereof or said Munc13-3 or (a) part(s) or (a) fragment (s).

Furthermore, a method for identifying and/or obtaining a isoform-specific modulator of Munc-13-3 activity is provided herein which comprises the steps of: a) contacting a Munc-13-3 molecule or (a) part(s) or (a) fragment(s) thereof with a candidate molecule; b) measuring and/or detecting a response; and c) comparing said response to a standard response as measured in the absence of said candidate molecule; d) contacting the identified and/or obtained candidate molecule of step c) with Munc13-1 or (a) part(s) or (a) fragment(s) thereof or Munc13-2 or (a) part(s) or (a) fragment(s) thereof; e) measuring and/or detecting whether said candidate molecule interacts with said Munc13-1 or (a) part(s) or (a) fragment(s) thereof or said Munc13-2 or (a) part(s) or (a) fragment(s) thereof; and f) selecting a candidate molecule which is not capable of interacting with Munc13-1 or (a) part(s) or (a) fragment(s) thereof or with Munc13-2 or (a) part(s) or (a) fragment(s).

The term “isoform-specific modulator” as employed herein relates to a modulator, for example an inhibitor or an activator which is capable of selectively interacting with and/or interfering with the specific Munc13 isoform. Said modulator may be a partial or a complete activator or inhibitor of Munc13-isofom activity. The activity of Munc13 may be measured by in vitro as well as by in vivo methods, as, inter alia, shown in the appended examples.

The term “activity” as used herein above in context of the method of the invention also comprises the “function” of the Munc13/Munc13 isoforms. Said function may comprise, as mentioned herein above, enzymatic activities or other functions, like, inter alia, involvement in signaling pathways, changes in intracellular localization, changes in vesicular release probability or vesicle priming. Such activities and modulators of such activities may be determined and/or identified by convenient in vitro or in vivo assays as described herein or by variations thereof. The underlying technology is widely and commonly known to the person skilled in the art.

The term “a molecule or (a) part or (a) fragment thereof” in context of this invention and in relation to the Munc13 isoforms Munc 13-1, Munc 13-2 or Munc 13-3 relate to the Munc13-molecules as known in the art and preferably relate to Munc13-isoforms of mouse, rat or human. These molecules are, inter alia, described in Brose (1995) or Betz (1996) Biochem. Soc. Trans. 24, 661-666. The term “part or fragment of Munc 13-isoforms” relate to partial Munc13 polypeptides which may comprise N-terminal as well as C-terminal domains, C1-domains or C2-domains or parts thereof. Furthermore combinations of parts of Munc 13 isofoms are envisaged in context of this invention, for example a combination of C1-domain with the N-terminal domain or the C-terminal domain of Munc13 isoforms.

The term “Munc13-1, Munc13-2 or Munc13-3 molecule” as used herein relates to specific (expressed) polypeptides as described herein below but also relates to mutated versions of said Munc13-isofoms. Such mutations may comprise deletions, substitutions (as for example documented in the appended examples), additions, inversions and the like. Mutated versions of the Munc13 isoforms may be physiologically active or inactive. Inactive versions may, inter alia, be employed in controls for the method of the present invention. The activity of Munc13-isoforms (Munc13s) may be measured by methods shown in the appended examples, in particular be elctrophysiological measurements. The term “Munc13-1, 13-2 and 13-3” also relate to naturally occurring and/or genetically engineered variants of said Munc13 isoforms and also comprise chemically modified and/or labeled molecules. Said variants may, e.g. comprise allelic variants or splice variants. It is also envisaged that fusionproteins are employed in context of this invention, whereby said fusion protein may comprise the full-length Munc-isoform or a part or a fragment thereof and at least one further domain besites said Munc13 isoform or fragment thereof. Said further domain maybe linked to the Munc13 isoform or to its fragment by covalent or non-covalent bonds. The linkage can be based on genetic fusion according to the methods known in the art (Sambrook, loc. cit., Ausubel, “Current Protocols in Molecular Biology”, Green Publishing Associates and Wiley Interscience, N.Y. (1989)) or can be performed by, e.g., chemical cross-linking as described in, e.g., WO 94/04686. The additional domain present in the fusionprotein comprising the Munc13 isoform or its fragment may preferably be linked by a flexible linker, advantageously a (poly)peptide linker, wherein said (poly)peptide linker preferably comprises plural, hydrophilic, peptide-bonded amino acids of a length sufficient to span the distance between the C-terminal end of said further domain and the N-terminal end of the Munc13 isoform or its fragment or vice versa. The above described fusionprotein may further comprise a cleavable linker or cleavage site, which, for example, is specifically recognized and cleaved by proteinases or chemical agents.

Additionally, said at least one further domain may be of a predefined specificity or function. In this context, it is understood that the Munc13 isoforms to be employed in the method of this invention may be further modified by conventional methods known in the art. This allows for the construction of fusionproteins comprising the proteins/protein fragments of Munc13 isoforms and other functional amino acid sequences, e.g., (vesicle or plasma-membrane) localization signals, transactivating domains, hormone-binding domains, protein tags (e.g. GST, GFP, h-myc peptide, FLAG, HA peptide, Strep), transmembrane domains or fatty acid attachment motifs, etc. which may be derived from heterologous proteins. In a preferred embodiment the fusionprotein of the Munc13 isoform to be employed in the method of the invention comprises at least one C1 of said Munc13 isoform. Yet, it is also envisaged that the highly variable N-terminal or C-terminal regions of Munc13-isoforms are employed in the method of the present invention or that these regions are comprised in the fusionproteins.

The unc13/Munc13 isoform or its fragment to be employed in the method of the invention may also be detectably labeled. A variety of techniques are available for labeling biomolecules, are well known to the person skilled in the art and are consitered to be within the scope of the present invention. Such techniques are, e.g., described in Tijssen, “Practice and theory of enzyme immuno assays”, Burden, R H and von Knippenburg (Eds), Volume 15 (1985), “Basic methods in molecular biology”; Davis L G, Dibmer M D; Battey Elsevier (1990), Mayer et al., (Eds) “Immunochemical methods in cell and molecular biology” Academic Press, London (1987), or in the series “Methods in Enzymology”, Academic Press, Inc. There are many different labels and methods of labeling known to those of ordinary skill in the art. Examples of the types of labels which can be used in the present invention include enzymes, radioisotopes (like ³²P or ¹²⁵I), colloidal metals, fluorescent compounds/fluorochromes (like fluorescein, rhodamine, Texas Red, etc.), chemiluminescent compounds, and chemi- or bioluminescent compounds (like dioxetanes, luminol or acridiniums). Commonly used labels furthermore comprise, inter alia, enzymes (like horse radish peroxidase, β-galactosidase, alkaline phosphatase), biotin or digoxygenin. Labeling procedures, like covalent coupling of enzymes or biotinyl groups, iodinations, phosphorylations, biotinylations, random priming, nick-translations, tailing (using terminal transferases) are well known in the art. Detection methods comprise, but are not limited to, autoradiography, fluorescence microscopy, direct and indirect enzymatic reactions, etc.

The term “contacting a Munc13 isoform with a a candidate molecule” may comprise tests of interaction. Such tests may be carried out by specific immunological and/or biochemical assays which are known in the art and comprise homogenous and heterogeneous assays as described herein and in the appended examples. For example, in the method of the present invention, the interaction assays to be employed in accordance with this invention may be used to detect as a response the direct or indirect interaction of the unc13/Munc13 isoform or its fragment or part with said candidate molecule. Said interaction assays employing read-out systems are well known in the art and comprise, inter alia, two hybrid screenings (as, described, inter alia, in EP-0 963 376, WO 98/25947, WO 00/02911), GST-pull-down columns, co-precipitation assays from cell extracts as described, inter alia, in Kasus-Jacobi, Oncogene 19 (2000), 2052-2059, “interaction-trap” systems (as described, inter alia, in U.S. Pat. No. 6,004,746) expression cloning (e.g. lamda gtlI), phage display (as described, inter alia, in U.S. Pat. No. 5,541,109), in vitro binding assays and the like. Further interaction assay methods and corresponding read out systems are, inter alia, described in U.S. Pat. No. 5,525,490, WO 99/51741, WO 00/17221, WO 00/14271, WO 00/05410. Said interaction assays may also comprise FRET-assays, TR-FRETs (in “A homogenous time resolved fluorescence method for drug discovery” in: High throughput screening: the discovery of bioactive substances. Kolb, (1997) J. Devlin. NY, Marcel Dekker 345-360) or commercially available assays, like “Amplified Luminescent Proximity Homogenous Assay”, BioSignal Packard.

Similarly, interacting candidate molecules may be deduced by cell-based techniques well known in the art. These assays comprise, inter alia, the expression of reporter gene constructs or “knock-in” or “knock-out” assays. Said “knock-in/out” assays may comprise “knock-in/out” in tissue culture cells, as well as in (trangenic) animals (as documented in the appended example). It is, inter alia, envisaged that such “knock-in assays” comprise the expression of the unc13 or the Munc13-isoform or its part or its fragment. Examples for successful “knock-ins” are known in the art (see, inter alia, Tanaka, J. Neurobiol. 41 (1999), 524-539 or Monroe, Immunity 11 (1999), 201-212). As documented in the appended examples, several “Munc13-knock in” or “Munc13-knock out” transgenic animals have been generated. These animals are particularly useful for the methods described herein. Accordingly, the present invention also provides for the use of a transgenic animal (over)expressing unc13/Munc13 or a fragment thereof or of a transgenic animal which does not express unc13/Munc13 for identifying and/or obtaining a molecule which is capable of modifying secretion processes or for identifying and/or obtaining an isoform-specific modulator of Munc13-1-, Munc13-2- or Munc13-3-activity. It is, e.g., envisaged that transgenic, non-human animal (like mice) express Munc13-1, Munc13-2 and/or Munc13-3 (“knock-in animals”) and are employed in the methods provided in this invention. A particularly useful transgenic animal for the specific methods of the present invention is the “knock-in” Munc13-1 mouse described in the appended examples. Similarly, it is of note that also “knock-out” transgenic, non-human animals may be employed in accordance with the methods of this invention. A specific example of such a “knock-out” transgenic animal is the Munc13-2 knock-out mouse described herein. Furthermore, biochemical assays may be employed which comprise, but are not limited to, binding of the Munc13 isoforms (or (a) fragment(s) thereof) to other molecules/(poly)peptides, peptides and assaying an interactions by, inter alia, immuno-precipitation assays, scintillation proximity assay (SPA), homogenous time-resolved fluorescence assay (HTRFA) and the like. The interacting candidate molecule may also be assessed by immuno-assays known in the art, for example ELISA, RIA, IRMA, FIA, CLIA, ECL, etc.

It is understood that the methods as disclosed herein also comprise physiological assays, in particular electrophysiological assays as documented in the appended examples. For example, it is envisaged in context of this invention that the Munc13 isoforms or its fragments are expressed in host cells and that said host cells are contacted with the candidate molecule to be tested. Said host cells may be prokaryotic as well as eukaryotic cells and may comprise bacterial, fungal, plant as well as animal cells. Said cells may also be cultured neuronal cells, like hippocampal neurons, but may also be common culture cell like PC12, CHO, HeLA and the like. Yet, the invention also envisages that Munc13 isoforms are expressed in cultured tissue, for example brain slices, and said the above described test for interaction and/or physiological response is carried out in vitro on said cultured tissue. As mentioned herein above and as documented in the appended examples, it is envisaged that the Munc13 isoforms are expressed in transgenic, non-human test animals and that the corresponding interaction and/or physiological tests are carried out on said test animals or on tissue or cells derived from said animals.

Further method(s) which may be employed comprises FRET (fluorescence resonance energy transfer; as described, inter alia, in Ng, Science 283 (1999), 2085-2089 or Ubarretxena-Belandia, Biochem. 38 (1999), 7398-7405), or fluorescence polarization assays. These methods are well known in the art and inter alia described in Fernandez, Curr. Opin. Chem. Biol. 2 (1998), 547-603.

Said “testing of interaction” may also comprise the measurement of a complex formation. The measurement of a complex formation is well known in the art and comprises, inter alia, heterogeneous and homogeneous assays. Homogeneous assays comprise assays wherein the binding partners remain in solution and comprise assays, like agglutination assays. Heterogeneous assays comprise assays like, inter alia, immuno assays, for example, ELISAs, RIAs, IRMAs, FIAs, CLIAs or ECLs.

The term “contacting a Munc13-isoform or (a) part(s) or (a) fragment(s) thereof with a candidate molecule” also relates to contacting a host carrying an expression vector comprising a nucleic acid molecule encoding for a Munc13-isoform or (a) part(s) or (a) fragment(s) thereof and operatively linked to a readout system with a compound or a collection of compounds, i.e. the candidate molecule. It may then be assayed whether said contacting results in a change of signal intensity provided by said readout system, and, optionally, identifying an individual compound within said collection of compounds that induces a change of signal. The change of signal may be then correlated with a change in the activity of Munc13-isoform or the expression of said Munc13-isoform. The host comprising/carrying said expression vector may be a eukaryotic cell, preferably a mammalian, most preferably a human cell. Said cell may be a neural cell. Yet, said host cell may also be a prokaryotic cell, e.g. a bacterium as well as an animal, like a transgenic non-human animal.

The candidate molecule to be tested in the method of the present invention may be a single isolated substance as well as a plurality of substances which may or may not be identical. Said candidate molecules/compound(s) may be comprised in, for example, samples, e.g., cell extracts from, e.g., plants, animals or microorganisms. Furthermore, said compound(s) may be known in the art but hitherto not known to be capable of influencing the activity of unc13 or of Munc13 isoforms or not known to be capable of influencing the expression of the nucleic acid molecule encoding a Munc13 isoform, respectively. The plurality of compounds may be, e.g., added to a sample in vitro, to the culture medium or injected into the cell or a test animal, preferably a transgenic test animal.

If a sample (collection of candidate molecules) containing (a) compound(s) is identified in the method(s) of the invention, then it is either possible to isolate the compound from the original sample identified as containing the compound in question or one can further subdivide the original sample, for example, if it consists of a plurality of different compounds, so as to reduce the number of different substances per sample and repeat the method with the subdivisions of the original sample. It can then be determined whether said sample or compound displays the desired properties by methods known in the art such as described herein. Depending on the complexity of the samples, the steps described above can be performed several times, preferably until the sample identified according to the method of the invention only comprises a limited number of or only one substance(s). Preferably said sample comprises substances of similar chemical and/or physical properties, and most preferably said substances are identical. The methods of the present invention can be easily performed and designed by the person skilled in the art, for example in accordance with other cell based assays described in the prior art (see, e.g., EP-A-0 403 506). Furthermore, the person skilled in the art will readily recognize which further compounds and/or cells may be used in order to perform the methods of the invention, for example, host cells as described herein above.

Compounds/Candidate molecules which can be used in accordance with the method of the present invention include, inter alia, peptides, proteins, antibodies (for example intracellular antibodies), aptamers, intramers or small organic compounds may be employed as candidate molecules in the method of the present invention. It is also envisaged that the methods described herein are employed to detect specific inhibitors or activators of Munc13-isoform gene expression. Accordingly, the method for identifying and/or obtaining a isoform-specific modulator of Munc13-isoforms and/or their activity also relates to enhancers or silencers of gene expression as well as to antisense molecules or ribozymes. Accordingly, the term “contacting a Munc13 molecule or (a) part or (a) fragment thereof with a candidate molecule” is not limited to the interaction of a Munc13-isofom polypeptide with a candidate molecule but also relates to a polynucleotide coding for said Munc13-isoform. Said polynucleotide may comprise coding as well as non-coding regions and may comprise promoter, enhancer or silencer regions.

Said compounds/candidate molecules can also be functional derivatives or analogues of known activators or inhibitors. Methods for the preparation of chemical derivatives and analogues are well known to those skilled in the art and are described in, for example, Beilstein, loc. cit. Furthermore, said derivatives and analogues can be tested for their effects according to methods known in the art and/or as described herein. Furthermore, peptidomimetics and/or computer aided design of appropriate activators or inhibitors of the expression of the nucleic acid molecules coding for Munc13 isoforms or of the activity of Munc13 isoforms can be used, for example, according to the methods described herein. Appropriate computer systems for the computer aided design of, e.g., proteins and peptides are described in the prior art, for example, in Berry, Biochem. Soc. Trans. 22 (1994), 1033-1036; Wodak, Ann. N.Y. Acad. Sci. 501 (1987), 1-13; Pabo, Biochemistry 25 (1986), 5987-5991. The results obtained from the above-described computer analysis can be used in combination with the method of the invention for, e.g., optimizing known compounds, substances or molecules. Appropriate compounds/candidate molecules can also be identified by the synthesis of peptidomimetic combinatorial libraries through successive chemical modification and testing the resulting compounds, e.g., according to the methods described herein. Methods for the generation and use of peptidomimetic combinatorial libraries are described in the prior art, for example in Ostresh, Methods in Enzymology 267 (1996), 220-234 and Dorner, Bioorg. Med. Chem. 4 (1996), 709-715. Furthermore, the three-dimensional and/or crystallographic structure of inhibitors or activators Munc13 isoforms can be used for the design of peptidomimetic inhibitors or activators of the Munc13-isoforms or the fusionprotein described herein and to be tested in the method of the invention (Rose, Biochemistry 35 (1996), 12933-12944; Rutenber, Bioorg. Med. Chem. 4 (1996), 1545-1558).

Candidate agents/candidate compounds to be tested in the methods of the present invention may also encompass numerous chemical classes, though typically they are organic molecules, preferably small organic compounds having a molecular weight of more than 100 and less than about 2,500 daltons. Candidate agents/compounds comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides. Candidate agents are obtained from a wide variety of sources including libraries of synthetic or natural compounds. In a preferred embodiment, the candidate agents are organic chemical moieties, a wide variety of which are available in the literature.

A wide variety of assays for binding agents/candidate agents/candidate compounds are provided including labeled in vitro protein-protein binding assays, immunoassays, cell based assays, etc. The methods are amenable to automated, cost-effective high-throughput screening of chemical libraries for lead compounds and have immediate application in a broad range of domestic and international pharmaceutical and biotechnology drug development programs. Identified reagents find use in the pharmaceutical industries for animal and human trials; for example, the reagents may be derivatised and re-screened in vitro and in vivo assays to optimize activity and minimize toxicity for pharmaceutical development.

In vitro binding assays employ a mixture of components including a complex or fusionprotein of the invention, which may be part of a fusion product with another peptide or (poly)peptide(s), e.g. a tag for detection or anchoring, etc. The complex or fusionprotein of the invention or fragment(s) thereof used in the methods are usually added in an isolated, partially pure or pure form and are typically recombinantly produced. The assay mixture also comprises a candidate pharmacological agent at different concentrations.

Candidate agents encompass numerous chemical classes, though typically they are organic compounds; preferably small organic compounds. Small organic compounds have a molecular weight of more than 50 Da yet less than about 2,500 Da, preferably less than about 1,000 Da, more preferably, less than about 500 Da. Candidate agents comprise functional chemical groups necessary for structural interactions with proteins and/or DNA, and typically include at least an amine, carbonyl, hydroxyl or carboxyl group, preferably at least two of the functional chemical groups, more preferably at least three. The candidate agents often comprise cyclical carbon or heterocyclic structures and/or aromatic or polyaromatic structures substituted with one ore more of the aforementioned functional groups. Candidate agents are also found among biomolecules including peptides, saccharides, fatty acids, steroids, purine, pyrimidies, derivatives, structural analogues or combinations thereof, and the like. Where the agent is or is encoded by a transfixed nucleic acid, said nucleic acid is typically DNA or RNA. As mentioned herein above, the candidate compound itself may be “nucleic acid molecule, e.g. a DNA or an RNA encoding a potential candidate or inhibiting the expression of a Munc13-isoform or unc13. Such inhibiting nucleic acid molecules comprise, inter alia, antisense oligonucleotides, RNAi and the like.

Candidate agents are obtained from a wide variety of sources including libraries of synthetic or natural compounds. For example, numerous means are available for random and directed synthesis of a wide variety of organic compounds and biomolecules. Alternatively, libraries of natural compounds in the form of bacterial, fungal, plant, and animal extracts are available or readily produced. Additionally, natural and synthetically produced libraries and compounds are readily modified through conventional chemical, physical, and biochemical means. In addition, known pharmacological agents may be subject to directed or random chemical modifications, such as acylation, alkylation, esterification, amidification, etc., to produce structural analogues.

The methods described herein are particularly suited for automated high-throughput drug screening using robotic liquid dispensing workstations. Similar robotic automation is available for high-throughput cell plating and detection of various assay read-outs.

Generally a plurality of assay mixtures are run in parallel with different agent concentrations to obtain a differential response to the various concentrations. Typically, one of these concentrations serves as a negative control, i.e. at zero concentration or below the limits of assay detection.

After incubation, the agent-biased binding and/or affinity between the Munc13 isoform and one or more binding targets o the agent-biased phsyiological response (e.g. electrophysiological response) is detected by any convenient way. For cell-free binding type assays, a separation step is often used to separate bound from unbound components. The separation step may be accomplished in a variety of ways. Conveniently, at least one of the components, e.g the Munc13-isoform molecule is immobilized on a solid substrate, which may be any solid from which the unbound components may be conveniently separated. The solid substrate may be made of a wide variety of materials and in a wide variety of shapes, e.g. microtiter plate, microbead, dipstick, resin particle, etc. The substrate is chosen to maximize the signal to noise ratios, primarily to minimize background binding, for ease of washing and cost.

Separation may be effected for example, by removing a bead or a dipstick from a reservoir, emptying or diluting a reservoir such as a microtiter plate well, rinsing a bead (e.g. beads with iron cores may be readily isolated and washed using magnets), particle, chromatographic column or filter with a wash solution or solvent. Typically, the separation step will include an extended rinse or wash or a plurality of rinses and washes. For example, where the solid substrate is a microtiter plate, the wells may be washed several times with a washing solution, which typically includes those components of the incubation mixture that do not participate in specific binding such as salts, buffer, detergent, non-specific protein, etc.

As mentioned herein above, cell-free binding type assays may be performed in homogeneous formats that do not require a separation step, e.g. scintillation proximity assay (SPA), homogenous time-resolved fluorescence assay (HTRFA) and the like.

A difference in the binding affinity of Munc13 isoform to the target in the absence of the candidate agent as compared with the binding affinity in the presence of the agent indicates that the agent modulates the Munc13 isoform to the target. Similarly, a difference in the physiological response, for example of the electrophysiological response, in absence or in presence of the candidate molecule to be tested for specific modulation of Munc13-isoform activity indicates that the candidate agent/molecule/compound is capable of modifying the Munc13-isoform activity. The difference, as used herein, is statistically significant and preferably represents at least a 50%, more preferably at least a 90% difference Analogously, in cell-based assays, a difference Munc13-isoform-specific activity in the presence and absence of an agent indicates the agent modulates Munc13-iosform specific activity. Such cell-based approaches may involve transient or stable expression assays. In this method, cells are transfected with one or more constructs encoding in sum, a polypeptide comprising a portion of the Munc13 and a reporter under the transcriptional control of an Munc13-isoform responsive promoter. Alternatively, the Munc13-isoform specific promoter itself may be linked to a suitable reporter gene, e.g. luciferase, and used in cell-based assays to screen for compounds capable of modulating, via up- or down-regulation, Munc13-isoform specific expression.

The above mentioned comparison between the response upon contacting the unc13 molecule/the Munc13-isoform molecule with said candidate molecule and the standard response as measured in the absence of said candidate molecule may proved for the presence, the absence, the decrease or the increase of a specific signal in the readout system. Said readout system, as described herein may be a, e.g., a biochemical or a physiological readout system, like a electrophysiological readout system. Genetical readout systems are also envisaged. A specific signal which is increased over the standard signal/response may thereby be classified as being an activator of unc13/Munc13-isoform function or expression, whereas a decreased signal may be classified as being diagnostic for an inhibitor of unc13/Munc13-isoform function or expression.

Candidate agents shown to modulate the expression of the nucleic acid molecules encoding Munc13 isoforms or to modulate the activity of Munc13 isoforms provide valuable reagents to the pharmaceutical industries for animal and human trials. Target therapeutic indications are limited only in that the target Munc13 isoform be subject to modulation. In particular, candidate agents obtained from drug screening assays and the subject compositions provide therapeutic applications in diseases associated with modified transmitter release and or disorders of secretory pathways, as discussed herein below.

Accordingly, and in a most preferred embodiment, the molecule which is capable of modifying secretion processes and/or which is an isoform-specific modulator of Munc13 activity is to be employed in diagnostic or pharmaceutical compositions and, preferably, does not interact with protein kinases C (PKCs). Therefore, the present invention also relates to a methods described herein above which further comprises the steps of a) contacting the identified and/or obtained candidate molecule with (a) protein kinase(s) C or (a) part(s) or (a) fragment(s) thereof; b) measuring and/or detecting whether said candidate molecule interacts with said (a) protein kinase(s) C or (a) part(s) or (a) fragment(s) thereof; and c) selecting a candidate molecule which is not capable of interacting with (a) protein kinase(s) C or (a) part(s) or (a) fragment(s) thereof.

The term “not capable of interacting with a) protein kinase(s) C or (a) part(s) or (a) fragment(s) thereof” is not limited to a (direct) physical interaction but also comprises physiological events, like the activation of a PKC specific signal pathway.

As the PKC specific pathway has been implicated in cancerogenic activity, such compound activating or modulating PKC-specific pathway are no desirable. Because of the specific role of Munc13 isoforms in secretion, such undesirable effects are not expected.

The present invention also provides for a method for identifying and/or obtaining a molecule which is capable of modifying secretion processes or which is a Munc13-isoform specific modulator and which further comprises the steps of
(a) contacting the identified and/or obtained candidate molecule with a calmodulin-binding site;
(b) measuring and/or detecting whether said candidate molecule interacts with said calmodulin-binding site; and
(c) selecting the candidate molecule which is capable of interacting with said calmodulin-binding site.

The term “calmodulin-binding site” as employed herein refers to a specific amino acid sequence which is capable of interacting with calmodulin, the ubiquitous intracellular signaling molecule. Such calmodulin-binding sites are known in the art and may be deduced by methods described in the appended examples. Preferably, the “calmodulin-binding sites” to be employed in context of this invention is a “calmodulin-binding site” of a unc-13-molecule or a Munc13-isoform molcule. As shown in the appended examples preferred calmodulin binding sites are “calmodulin-binding sites” of Munc13-1, ubMunc13-2, DUNC-13 or unc-13 of C. elegans.

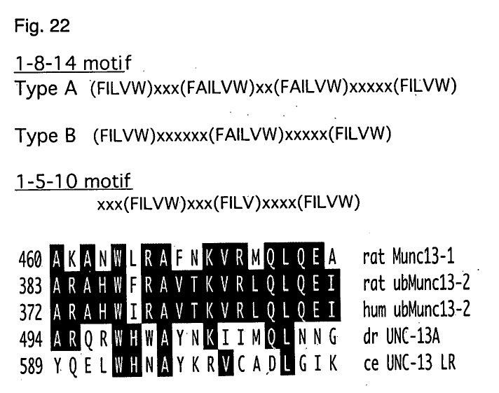

Further preferred “calmodulin binding sites” comprise an amino acid sequence as shown in SEQ ID NOs: 11 or 12 or as depicted in appended FIG. 22.

As documented in the appended examples, Munc13-1 and one of the two alternative splice variants of Munc13-2 (ubMunc13-2) contain a calmodulin binding site in the n-terminal region. These calmodulin binding sites correspond to amino acid residues 460 to 478 in SEQ ID NO: 3 (in particular W464), amino acid residues 383 to 402 in SEQ ID NO: 6 (in particular W387), amino acid residues 372 to 390 in SEQ ID NO: 5 (in particular W376), amino acid residues 494 to 512 in SEQ ID NO: 9 (in particular W498) or amino acid residues 589 to 607 in SEQ ID NO: 10 (in particular W593). The brain-specific Munc13-2 isoform (bMunc13-2) as well as Munc13-3 does not contain this Calmodulin binding site. Furthermore this calmodulin binding site is present in the drosophila Dunc13 (amino acid residues 494 to 512, in particular W498, in SEQ ID NO: 9) and the C. elegans Unc13 (amino acid residues 589 to 607, in particular W593 in SEQ ID NO: 10).

Besites this general prediction, due to the exquisite regulation of Munc13 isoform distribution and due to its presence and absolute requirement for secretion in all neurons and neuroendocrine cells, isoform-specific agonists as well as antagonists may be useful in all therapeutic strategies that target hormone or neurotransmitter secretion or are based on altering informational processing in the brain.

Munc13-1 as well as ubMunc13-2 (of rat and human) were shown to increase their enzymatic activity (vesicle priming) when primed vesicles are depleted upon usage (see examples described herein). This allows a sufficient number of primed vesicles in the nerve terminal to allow for stable synaptic responses in trains of action potentials. As documented in the appended examples, it was surprisingly shown that binding of calmodulin to Munc13-1 and ubMunc13-2 is necessary for the activity dependent upregulation of Munc13 function. The trigger for Calmodulin/Munc13 interaction is the elevation of intracellular calcium during the train of action potentials. In case of Munc13, it was surprisingly found that already a partial interaction of calmodulin at low Ca²⁺, while increases of calcium strenghtens the binding of Ca²⁺/calmodulin. In case of ubMunc13-2 Calmodulin binds only in its Calcium-bound state.

The importance of the Calmodulin/Munc13 interaction is reflected in the finding that the mutation of the Calmodulin binding site in ubMunc13-2 (W387R; as shown in SEQ ID NO: 6 or W376R as shown in SEQ ID NO: 5) converts synaptic behavior from a strong potentiation during trains of action potentials to rapid depression of the synaptic response. A similar principle can be seen when the same site is mutated in Munc13-1, although the overall effect is less pronounced. A similar behavior can also be seen when in any of the Munc13 isoforms the DAG binding C-1 region is mutated.

Given the different sequences of Munc13 calmodulin binding sites in the different isoforms, the present invention allows for a method of screening for substances which are (Munc13-/Unc13) isoform specific ligands for the calmodulin binding site that either mimick the effect of Ca2+/Calmodulin binding (agonists, allows for synaptic potentiation) or that are able to prevent Munc13 or Unc13 to respond to activity dependent potentiation (antagonists). Furthermore based on the region- and synapse-specific distribution of Munc13-isoforms treatment of these agonists and antagonists may be used to obtain a selective regulation of a particular synapse population within the brain or a particular class of hormone secreting cells in the body.

Application of agonists of the Ca²⁺/calmodulin binding site of Munc13 or Unc13 lead to a tonic potentiated state of Munc13, independent of the activity state of the cell. Without being bound by theory, this leads to an enhancement of synaptic responses, and a better performance of synapses particularly when action potentials arrive at low frequencies at the synapse. Potential treatments with Munc13-Calmodulin binding site agonists may involve neurological states that have a general weakened synaptic activity either due to loss of synaptic connections or through loss of neurons. Depending on the class of neurons and connections affected, this could be used for treatment of, inter alia, Huntigtons disease, Alzheimer's disease, Parkinsons disease, ALS, and conditions of imbalanced relationships between excitation and inhibition with neuronal networks. Furthermore, many mood disorders or sleep disorders are characterized by reduced or imbalanced activity of certain neuronal pathways, and a stimulation of specific pathway through these agonist may be counteract these syndromes. Outsite of the brain any disease related to hypofunction of of secretory cells may be applicable such as diabetes mellitus, or hypothyroidism. Antagonists of the here described calmodulin-binding site of Munc13/Unc13 would in general prevent synapses to maintain high synaptic activity when neurons are highly excited or fire at high action potential frequencies. This would not lead to a dampening of synptic transmission at low activity, but prevent in particular overexcitation. Diseases that may be treated with antagonists of the calmodulin-binding site of Munc13/Unc13 are, inter alia, disorders of increased or imbalanced activity of certain neuronal pathways such as occurring at certain forms of epilepsy and/or the treatment of ischemic states. Furthermore, any disease state with secretory cell or gland function such as hypertyhroidism may be treatable with such antagonists.

As mentioned herein above, preferably the unc-13 molecule to be employed in the method of the invention is selected from group consisting of Xenopus Unc-13, Drosphila dUnc-13, C. elegans Unc13, mouse Munc-13, rat Munc13, human Munc13. Most preferably said mouse, rat or human Munc-13 is Munc13-1, Munc-13-2 or Munc13-3.

Munc13-isoforms are known in the art. For example, the Munc13-isoforms or the part/fragment thereof to be employed in this invention may be encoded by a polynucleotide comprising a nucleotide sequence as shown in GenBank Accession number: AB028955 (human Munc13-1 cDNA), AF020202 (human ubiquitous Munc13-2), AK010728 (mouse Munc13-1 cDNA), AF115848 (mouse ubiquitous Munc13-2), U24070 (rat Munc13-1), U24071 (rat brain Munc13-2), AF159706 (rat ubiquitous Munc13-2), U24072 (rat Munc13-3), NM_{—}059692 (C. elegans Unc-13 LR) or NM_{—}059693 (C. elegans Unc-13 MR). The person skilled in the art can readily deduce further Munc13 sequences, in particular the different isoforms by methods known in the art, like, inter alia, PCR methods. It is of note that also mutated Munc13 isoforms may be employed in the method of the present invention. Employing techniques as illustrated in the appended examples, it is easily established, whether a mutated version of the Munc13 isoforms is functional. The surprising results documented in said examples allow the distinction between different Munc13 isofoms, e.g. by electrophysiological means.

Preferably, said Munc13-1 may be encoded by a nucleic acid molecule encoding a polypeptide as shown in SEQ ID NO: 1 (partial protein sequence of mouse Munc13-1), SEQ ID NO: 2 (partial protein sequence of human Munc 13-1) or SEQ ID NO.3 protein sequence of rat Munc13-1).

Preferably, said Munc13-2 may be encoded by a nucleic acid molecule encoding a polypeptide as shown in SEQ ID NO.: 4 (ubiquitous mouse Munc 13-2 polypeptide), SEQ ID NO: 5 (ubiquitous human Munc13-2 polypeptide), SEQ ID NO: 6 (ubiquitous rat Munc13-2 polypeptide) or SEQ ID NO: 7 (brain-specific rat Munc13-2 polypeptide).

Preferably, said Munc13-3 is encoded by a nucleic acid molecule encoding a polypeptide as shown in SEQ ID NO.: 8 (rat Munc13-3 polypeptide)

An example of a Drosophila Unc13 (DUNC-13) is shown in SEQ ID NO: 9, whereas SEQ ID NO: 10 depicts an exemplified amino acid sequence of C. elegans Unc13.

However, and as mentioned herein above, isoforms and variants of Munc13 isoforms may be employed in accordance with this invention. Said variants comprise may. Inter alia, allelic isofoms or tissue-specific isoforms. Furthermore, variants, like splice-variants of the Munc13s may be employed in the methods of the present invention or in the pharmaceutical or diagnostic compositions described herein. It is also envisaged that Munc13-variants, like specific mutations, are employed in the methods of the invention. Examples for such mutations comprise the specific exchange of a tryptophan to arginine; for example it is envisaged that a W464R-mutation in SEQ ID NO: 3, a W387R mutation in SEQ ID NO: 6, a W376R mutation in SEQ ID NO. 5, a W498R mutation in SEQ ID NO: 9 or a W593R mutation in SEQ ID NO: 10 is employed in the methods or compositions of this invention. Said variants and isofoms may also be useful in the preparation of such pharmaceutical and diagnostic compositions and could be employed in the medical settings described herein below. Most preferably, the Munc-isoforms to be employed in accordance with this invention are more than 70%, more preferably more than 80%, more preferably more than 90% homologous to the polypeptides as encoded by the polynucleotides as shown in SEQ ID NOs:1 to 8.

Munc13 isoforms show an overall homology of about 70%, yet they are highly distinct in their N-terminal region and show also distinct differences in the C-terminus. However, also the C1-region is distinct and, accordingly, the person skilled in the art can easily deduce the C1-region from Munc13-1, Munc13-2 or Munc13-3, respectively.

In a preferred embodiment of the present invention, the part or fragment of unc-13 to be employed in the method of the invention comprises the C1-region of unc13. Accordingly, said part or fragment of Munc13-1, Munc13-2 or Munc13-3 comprises, preferably, the C1-region of Munc13-1, Munc13-2 or Munc13-3. Yet, and as mentioned herein above, it is also envisaged that the N-terminal, the C-terminal regions or the C2-regions of Munc13 isoforms, as well as combinations thereof are employed in the method of the present invention.

In a most preferred embodiment of the present invention, said molecule which is capable of modifying secretion processes or said isoform-specific modulator and to be identified and/or obtained is an antagonist or an agonist of unc13 expression or activity or of Munc13-isofrorm specific activity or expression.

It is, for example envisaged that an antagonist of Munc13/Unc13 is an inhibiting nucleic acid molecule.

Accordingly, potential antagonistic or inhibiting compounds comprise nucleic acid molecules that are capable of reducing Munc13/Unc13 activity in a cell by way of interfering the gene expression of Munc13/Unc13 and/or gene expression. Such antagonists/inhibitors are, inter alia, antisense oligonucleotides, antisense DNA, antisense RNA, iRNA, ribozymes or siRNA. Said inhibiting nucleic acids will be described herein below in more detail.

An inhibiting/antagonistic nucleic acid molecule is preferably complementary to any Munc13/Unc13 sequence, for example 5′-untranslated regulatory region, the open reading frame or 3′-untranslated region of Munc13-1, Munc13-2, Munc13-3 or Unc13 as described herein. Mutatis mutandis, these nucleic acid molecules may also target the corresponding region(s) of genes encoding Munc13/Unc13 proteins as defined herein. Said inhibiting nucleic acid molecules are preferably used for repression of expression of a gene comprising such sequences, for example due to an antisense or triple helix effect or for the construction of appropriate ribozymes (see e.g., EP-B1 0 291 533, EP-A1 0 321 201, EP-B1 0 360 257) which specifically cleave the (pre)-mRNA of a gene comprising a sequence of the Munc13/Unc13 gene or genes encoding Munc13/Unc13-isoforms. Selection of appropriate target sites and corresponding ribozymes can be done as described for example in Steinecke, Ribozymes, Methods in Cell Biology 50, Galbraith et al. eds Academic Press, Inc. (1995), 449-460.

The person skilled in the art can easily deduce corresponding nucleic acid sequences to be employed in this context, since the sequences for, inter alia, Munc13-1, Munc13-2, Munc13-3 from rat, mouse, human etc. are well known and shown in the appended sequence listing.

Antisense technology can be used to control gene expression through triple-helix formation or antisense DNA or RNA, whereby the inhibitory effect is based on specific binding of a nucleic acid molecule to DNA or RNA. For example, the 5′ coding portion of a nucleic acid molecule encoding a Munc13 or Unc13 to be inhibited can be used to design an antisense oligonucleotide, e.g., of at least 10 nucleotides in length. The antisense DNA or RNA oligonucleotide hybridises to the mRNA in vivo and blocks translation of said mRNA and/or leads to destabilization of the mRNA molecule (Okano, J. Neurochem. 56 (1991), 560; Oligodeoxynucleotides as antisense inhibitors of gene expression, CRC Press, Boca Raton, Fla., USA (1988)).

For applying a triple-helix approach, a DNA oligonucleotide can be designed to be complementary to a region of the gene encoding a Munc13-isoforms or Unc13 to be inhibited according to the principles laid down in the prior art (see for example Lee, Nucl. Acids Res. 6 (1979), 3073; Cooney, Science 241 (1988), 456; and Dervan, Science 251 (1991), 1360). Such a triple helix forming oligonucleotide can then be used to prevent transcription of the specific gene. The oligonucleotides described above can also be delivered to target cells via a gene delivery vector as described above in order to express such molecules in vivo to inhibit gene expression of the respective protein.

Examples for antisense molecules are oligonucleotides specifically hybridizing to a polynucleotide encoding a polypeptide having Munc13 or Unc13 activity. Such oligonucleotides have a length of preferably at least 10, in particular at least 15, and particularly preferably of at least 50 nucleotides. They are characterized in that they specifically hybridize to said polynucleotide, that is to say that they do not or only to a very minor extent hybridize to other nucleic acid sequences.

Another suitable approach is the use of nucleic acid molecules mediating an RNA interference (RNAi) effect. RNAi refers to the introduction of homologous double stranded RNA (dsRNA) to specifically target a gene's product, resulting in null or hypomorphic phenotypes. Introduction of dsRNA into a cell results in the loss of the targeted endogenous Munc13 isoforms or Unc13 mRNA as defined herein. Because RNAi is also remarkably potent (i.e., only a few dsRNA molecules per cell are required to produce effective interference), the dsRNA must be either replicated and/or work catalytically. Thereby, the formation of double-stranded RNA leads to an inhibition of gene expression in a sequence-specific fashion. More specifically, in RNAi constructs, a sense portion comprising the coding region of the gene to be inactivated (or a part thereof, with or without non-translated region) is followed by a corresponding antisense sequence portion. Between both portions, an intron not necessarily originating from the same gene may be inserted. After transcription, RNAi constructs form typical hairpin structures. In accordance with the method of the present invention, the RNAi technique may be carried out as described by Smith (Nature 407 (2000), 319-320) or Marx (Science 288 (2000), 1370-1372).

Likewise, RNA molecules with ribozyme activity which specifically cleave transcripts of a gene encoding a Munc13 isoform or Unc13 can be used. Said ribozymes may also target DNA molecules encoding the corresponding RNAs. Ribozymes are catalytically active RNA molecules capable of cleaving RNA molecules and specific target sequences. By means of recombinant DNA techniques it is possible to alter the specificity of ribozymes. There are various classes of ribozymes. For practical applications aiming at the specific cleavage of the transcript of a certain gene, use is preferably made of representatives of two different groups of ribozymes. The first group is made up of ribozymes which belong to the group I intron ribozyme type. The second group consists of ribozymes which as a characteristic structural feature exhibit the so-called “hammerhead” motif. The specific recognition of the target RNA molecule may be modified by altering the sequences flanking this motif. By base pairing with sequences in the target molecule these sequences determine the position at which the catalytic reaction and therefore the cleavage of the target molecule takes place. Since the sequence requirements for an efficient cleavage are low, it is in principle possible to develop specific ribozymes for practically each desired RNA molecule. In order to produce DNA molecules encoding a ribozyme which specifically cleaves transcripts of a gene encoding Munc13 isoforms or Unc13, for example a DNA sequence encoding a catalytic domain of a ribozyme is bilaterally linked with DNA sequences which are homologous to sequences encoding the target protein. Sequences encoding a catalytic domain and DNA sequence flanking the catalytic domain are preferably derived from the polynucleotides encoding Munc13 isoforms or Unc13. The expression of ribozymes in order to decrease the activity in certain proteins is also known to the person skilled in the art and is, for example, described in EP-B1 0 321 201 or EP-B1 0 360 257.

In a preferred embodiment, the inhibiting nucleic acid molecule is siRNA as dislosed in Elbashir ((2001), Nature 411, 494498)) and as illustrated in the appended examples.

It is also envisaged in accordance with this invention that for example short hairpin RNAs (shRNAs) are employed in accordance with this invention as anti-Munc13-isoform compounds, Munc13/Unc13 antagonists or Munc13/Unc13 inhibitors. The shRNA approach for gene silencing is well known in the art and may comprise the use of st (small temporal) RNAs; see, inter alia, Paddison (2002) Genes Dev. 16, 948-958.

As mentioned above, approaches for gene silencing are known in the art and comprise “RNA”-approaches like RNAi or siRNA. Successful use of such approaches has been shown in Paddison (2002) loc. cit., Elbashir (2002) Methods 26, 199-213; Novina (2002) Mat. Med. Jun. 3, 2002; Donze (2002) Nucl. Acids Res. 30, e46; Paul (2002) Nat. Biotech 20, 505-508; Lee (2002) Nat. Biotech. 20, 500-505; Miyagashi (2002) Nat. Biotech. 20, 497-500; Yu (2002) PNAS 99, 6047-6052 or Brummelkamp (2002), Science 296, 550-553. These approaches may be vector-based, e.g. the pSUPER vector, or RNA polIII vectors may be employed as illustrated, inter alia, in Yu (2002) loc. cit.; Miyagishi (2002) loc. cit. or Brummelkamp (2002) loc. cit.

Inhibiting molecules acting as Munc13 isoform specific antagonists/inhibitors may be introduced via gene therapy approaches (e.g. the introduction of heavy and/or light chains or at least the variable regions thereof or the introduction of scFvs by use of corresponding vector systems). Inhibiting RNAs as defined herein and to be employed as Munc13/Unc13 may also be introduced by vector systems and/or “gene therapy” approaches yet further introduction systems for the herein defined Munc13/Unc13 antagonists/inhibitors are envisaged. For example, liposomes, may also be employed in this context. Liposomes as transfection systems have been described in the art and have not only been employed for the introduction of genes and proteins/peptides but also for the transfection with oligonucleotides, like RNA; see, inter alia, Paul (2002) loc. cit.

20- to 50-nucleotide RNAs, preferably 15, 18, 20, 21, 25, 30, 35, 40, 45 and 50-nucleotide RNAs are chemically synthesized using appropriately protected ribonucleosite phosphoramidites and a conventional DNA/RNA synthesizer. Most conveniently, siRNAs and the like are obtained from commercial RNA oligo synthesis suppliers, which sell RNA-synthesis products of different quality and costs. In general, 20 to 50-nucleotide RNAs are not too difficult to synthesize and are readily provided in a quality suitable for RNAi. However, specific gene silcencing may also be obtained by longer RNA, for example long dsRNA which may comprise even 500 nt; see, inter alia, Paddison (2002) PNAS 99, 1443-1448. The preferred targeted region is selected from a given nucleic acid sequence beginning, inter alia, 50 to 100 nt downstream of the start codon.

Additionally, the present invention relates to a method of refining the compound or the agent identified by the method(s) described herein which, for example, modulates the activity of Munc13-isforms comprising (a) modeling said compound by peptidomimetics; and (b) chemically synthesizing the modeled compound.

Peptidomimetics is well known in the art and disclosed, inter alia, in Beeley, Trends Biotech 12 (1994), 213-216, Wiley, Med. Res. Rev. 13 (1993), 327-384, Hruby, Biopolymers 43 (1997), 219-266, or references cited therein. Methods of the generation and use of peptidomimetic combinatorial libraries are described in the prior art, for example in Ostresh, Methods in Enzymology 267 (1996), 220-234 and Dorner, Bioorg. Med. Chem. 4 (1996), 709-715. Methods for the chemical synthesis and/or the preparation of chemical derivatives and analogues are well known to those skilled in the art and are described in, for example, Beilstein, loc. cit. and “Organic Synthesis”, Wiley, New York, U.S.A., see supra.

The invention also relates to a composition comprising Munc13 polypeptide or a fragment thereof or a polynucleotide encoding a Munc13 polypeptide or a fragment thereof, comprising an antibody specifically detecting a Munc 13 polypeptide or comprising an antagonist or an agonist as identified and/or obtained by the method of the invention. Most preferred is a pharmaceutical or a diagnostic composition. It is of note that, inter alia, antibodies or antibody fragments or antibody derivatives specific for Munc13/Unc13 may function as isoform-specific modulators of Munc13 or as molecules which are capable of modidfying secretion processes. Accordingly, such antibodies, antibody fragments or antibody derivatives are envisaged to function as specific (e.g. isoform-specific) antagonists or agonists of Munc13/Unc13. Yet, also further compounds are envisaged as antagonists/agonists of Munc13/Unc13. These compounds, like the antibodies characterized herein may be tested by the methods described herein and illustrated in the appended examples, and said compounds may be, inter alia, selected from the group consisting of aptamers and small molecules.

As documented in the appended examples, it has surprisingly be found that Munc13-isoforms are highly divers in their physiological roles. Accordingly, a specific detection of Munc13-isoform expression or function, may be employed diagnostically. Therefore, polynucleotides encoding Munc13-isoforms or specific antibodies (of fragments or derivatives thereof) and the like may be employed to detect the expression level of Munc13-isoforms or mutations in the genes encoding Munc13-isoforms.

Such diagnostic methods may be carried out in vivo, ex vivo as well as in vitro.

It is, for example, envisaged that antibodies which specifically bind the unc13 and/or Munc13-isoforms may be used for the diagnosis of conditions or diseases described herein, or in assays to monitor patients being treated with the agonists, antagonists, activators or inhibitors as identified by the method of the present invention. The antibodies useful for diagnostic purposes (as well as in pharmaceutical and medical settings disclosed herein) may be prepared by methods known in the art. The general methodology for producing antibodies is well-known and has, for monoclonal antibodies, been described in, for example, Köhler and Milstein, Nature 256 (1975), 494 and reviewed in J. G. R. Hurrel, ed., “Monoclonal Hybridoma Antibodies: Techniques and Applications”, CRC Press Inc., Boco Raron, Fla. (1982). In accordance with the present invention the term “antibody” relates to monoclonal or polyclonal antibodies. Polyclonal antibodies (antiserum) can be obtained according to conventional protocols. Antibody fragments or derivatives comprise F(ab′)₂, Fab, Fv or scFv fragments; see, for example, Harlow and Lane, “Antibodies, A Laboratory Manual”, CSH Press 1988, Cold Spring Harbor, N.Y. Preferably the antibody of the invention is a monoclonal antibody. Antibodies in accordance with this invention also comprise humanized, chimeric antibodies, a CDR-grafted antibody, a bivalent antibody-construct, an antibody fusion protein, a scFv, a synthetic antibody and the like. In context of antibody molecules which function as inhibitors or activators of Munc13/Unc13 activity in context of this invention, it is preferred that said antibodies/antibody fragments or derivative may be expressed in cells or that said molecules are capable of transferring biological membranes or barriers, like the plasma membrane of cells or the blood-brain barrier of animals or humans.

Diagnostic assays for Munc13-isoforms include methods which utilize the antibody molecules described herein and a label to detect the said Munc13-isoform in human body fluids or extracts of cells or tissues. The antibodies or fragments or derivatives thereof may be used with or without modification, and may be labeled by joining them, either covalently or non-covalently, with a reporter molecule. A wide variety of reporter molecules which are known in the art may be used several of which are described above.

A variety of protocols including ELISA, RIA, and FACS for measuring the Munc13-isoforms are known in the art and provide a basis for diagnosing altered or abnormal levels of Munc13-isoforms or aberrant expression of said Munc13-isoforms. Normal or standard values for the Munc13-isoform expression are established by combining body fluids or cell extracts taken from normal mammalian subjects, preferably human, with antibody to the Munc13-isoforms under conditions suitable for complexes formation. The amount of standard Munc13-isofrom expression or activity may be quantified by various methods, but preferably by photometric, means. Quantities of the Munc13-isoforms expressed in control and disease samples from biopsied tissues are compared with the standard values. Deviation between standard and subject values establishes the parameters for diagnosing disease.

In another embodiment of the invention, the polynucleotides encoding the Munc13-isoforms may be used for diagnostic purposes. The polynucleotides which may be used include oligonucleotide sequences, antisense RNA and DNA molecules, and PNAs. The polynucleotides may be used to detect and quantitate gene expression in biopsied tissues in which expression of individual Munc13-isoforms may be correlated with disease. The diagnostic assay may be used to distinguish between absence, presence, and excess expression of the Munc13-isoforms, and to monitor regulation of the Munc13-isoforms levels during therapeutic intervention. The diagnostic composition of the invention provides for means of diagnosing and/or screening for a disorder or a disease associated with aberrant gene-expression of Munc13 isoforms or of aberrant Munc13-isoform activity. Said diagnostic composition is particularly useful in diagnosing a neurological or a secretarial disease or disorder as defined herein below or in detecting a predisposition for developing such a disease/disorder. The

The invention also provides for the use of an antagonist or an agonist of unc13 as identified and/or obtained by the method of claim 13 for the preparation of a pharmaceutical composition for the treatment of a neurological or a secretarial disorder or disease

Furthermore, the invention also relates to the use of a Munc13 polypeptide or a fragment thereof or a polynucleotide encoding a Munc13 polypeptide or a fragment thereof for the preparation of a pharmaceutical composition for the treatment of a neurological or a secretorial disorder or disease.

It is, inter alia, envisaged that specific Munc13-isoforms are to be expressed in a patient in need thereof. The pharmaceutical composition may, accordingly, also comprise nucleic acid molecules encoding the different Munc-13 isoforms or their parts or fragments as well as vectors or hosts comprising said nucleic acid molecules.

For therapeutic applications, in particular gene therapy applications, nucleic acids encoding the Munc13-isoforms described herein, may be cloned into a gene delivering system, such as a virus and the virus used for infection and conferring disease ameliorating or curing effects in the infected cells or organism. As mentioned herein above, the nucleic acid molecule(s) and/or vector(s) encoding Munc13-isoforms may be employed in order to modulate/alter the gene expression or intracellular concentration of said Munc13 isoforms. Said modulation/alteration may also be achieved by antisense-approaches. Gene therapy, which is based on introducing therapeutic genes into cells by ex-vivo or in-vivo techniques is one of the most important applications of gene transfer. Suitable vectors, methods or gene-delivering systems for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813, Isner, Lancet 348 (1996), 370-374; Muhlhauser, Circ. Res. 77 (1995), 1077-1086; Onodua, Blood 91 (1998), 30-36; Verzeletti, Hum. Gene Ther. 9 (1998), 2243-2251; Verma, Nature 389 (1997), 239-242; Anderson, Nature 392 (Supp. 1998), 25-30; Wang, Gene Therapy 4 (1997), 393-400; Wang, Nature Medicine 2 (1996), 714-716; WO 94/29469; WO 97/00957; U.S. Pat. No. 5,580,859; U.S. Pat. No. 5,589,466; U.S. Pat. No. 4,394,448, Geddes, Front Neuroendocrinol. 20 (1999), 296-316 or Geddes, Nat. Med. 3 (1997), 1402-1404 or Schaper, Current Opinion in Biotechnology 7 (1996), 635-640, and references cited therein. The nucleic acid molecules and vectors of encoding Munc13 isoforms may be designed for direct introduction or for introduction via liposomes, viral vectors (e.g. adenoviral, retroviral, lentiviral), electroporation, ballistic (e.g. gene gun) or other delivery systems into the cell. Additionally, a baculoviral system can be used as eukaryotic expression system.

In a preferred embodiment of the invention, the invention relates to the use of an antibody or a fragment or a derivative thereof, an receptor or an aptamer specifically detecting or binding to a Munc-13 molecule, in particular to a Munc13-isoform, for the preparation of a diagnostic composition for detecting a neurological or a secretorial disorder or disease. As mentioned herein above, it is also envisaged that receptors of Munc13/Munc13 isoforms, like antibodies or their fragments or derivatives as defined herein, aptamers, intramers and the like be employed in pharmaceutical settings. It is, for example, envisaged that said antibodies (or derivatives or fragments thereof) may be tested in accordance with the method of the present invention and may function as specific activators or inhibitors of Munc13/Munc13-isoforms.

The present invention also provides for a method for diagnosing a disease or disorder associated with a modified secretion process comprising the steps of
(a) measuring the expression level or activity of Unc13 or a Munc13-isoform in a tissue or cell sample; and
(b) correlating the measured expression level or activity with a reference sample representing a tissue, or cell sample not affected by said disease or disorder or with a reference sample representing a tissue- or cell sample affected by said disease or disorder.

Preferably, said disease or disorder to be diagnosed is a secretarial disease or disorder or is a neurological disease or disorder.

The diagnostic method of this invention may be carried out in vivo, ex vivo and most preferably in vitro.

The expression level of Unc13 or Munc13-isoforms may be determined by detecting the expressed protein (or fragments thereof), for example by immuno(histo)chemical methods like Western-Blotting or microscopical means (fluorescence-microscopy etc.). The activity of Unc13 or Munc13-isoforms may be measured by means described in the appended examples, e.g. electrophysiological methods. Samples to be used in the diagnostic methods described above may comprise samples from brains, liver, pancreas, kidney, thyroid gland.

In context of this invention, it is envisaged that the neurological disease or disorder to be diagnosed or treated is selected form the group consisting of schizophrenia, epilepsy, Parkinson's disease, Alzheimer's disease, Huntington's disease, (ischemic) or stroke and that said secretorial disease or disorder is selected from the group consisting of diabetes mellitus, Morbus Addison, hypothryodism, hyperthryodism, Morbus Cushing, hypertonus and diabetic nephropathy,

As mentioned herein above, the different Munc13 isoforms are expressed in different regions of the brain. For example, Munc 13-1 is expressed in all neurons of the nervous system. In contrast, Munc13-2 and munc13-3 exhibit strikingly different expression patterns. Munc13-2 is only present in the rostral brain regions, while Munc13-3 is mostly restricted to the cerebellum. Thus neurons coexpress Munc13-1 with either Munc13-2 or Munc13-3 depending on the brain region. Accordingly, with the surprising finding shown herein and illustrated in the appended examples, it is now feasible to specifically treat and diagnose neurological and secretorial disorders.

For example, agonists for Munc13-1 as identified and/or obtained by the method of the present invention lead to an overall increase of synaptic activity, especially in excitatory cells. This may be therapeutically be used to counteract diseases related to loss of brain function, e.g. Alzheimer, Parkinson's- and Huntington-disease. Hypthalamic dysfunction leading to reduced secretion of peptide hormones such as Hypothyriodism or Morbus Addison may be treated by Munc13-1 agonists, in particular agonists of the C1 domain. Antagonists of Munc13-1 activity or expression and as identified and/or obtained by the method of the present invention may be useful to counteract general pathopysiologically enhanced synaptic activity such as epilepsy, or during stroke or for hyperthyroidism.

Since Munc13-2 is mainly expressed in the forebrain and exhibits the surprising physiological features as disclosed herein, antagonist as well as agonists against this molecule, identifiable by the method disclosed herein, may be therapeutically employed. As schizophrenia is related to dysfunction of synaptic activity, both agonists and antagonists of Munc13-2 function may be used to counterbalance dysregulation of synaptic function. This may also be used to treat Parkinsons, Alzheimers and Huntington. Munc13-3 agonists and antagonists may be used to interfere with dysfunctions related to cerebellar or brain stem related diseases.

Yet, the agonists and antagonists of Munc13 and/or Munc13-isoforms may also be employed for the treatment of hormonal diseases, since, e.g. Munc13-1 and Munc13-3 are expressed in the adrenal glands (Asheri, 2001). Agonists against Munc13 may, therefore, be employed in the treatment of diseases related to adrenal hypofunctional (agonists). Antagonists may be employed in hyperfunctional diseases such as Morbus Addison, Morbus Cushing or hypertonus. ubMunc13-2 is expressed in kidney and in the pancreas and has been implicated in diabetic nephropathy. Diabetes mellitus may, inter alia, be treated with agonists of Munc13, in particular Munc13-2 function.

It is envisaged to employ in the pharmaceutical uses as disclosed herein inhibitors, activators, antagonists or agonists against Munc13 molecules. However, it is most preferred that isoform-specific inhibitors, activators, antagonists or agonists be employed.

The invention also provides for method for the preparation of a pharmaceutical composition comprising the steps of the method as disclosed and, additionally, formulating the identified and/or obtained molecule in a pharmaceutically acceptable form.

Examples of suitable pharmaceutical carriers, excipients and/or diluents are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical, intradermal or intranasal administration and the like. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. Proteinaceous pharmaceutically active matter may be present in amounts between 1 ng and 10 mg per dose; however, doses below or above this exemplary range are envisioned, especially consitering the aforementioned factors. If the regimen is a continuous infusion, it should also be in the range of 1 μg to 10 mg units per kilogram of body weight per minute, respectively. Progress can be monitored by periodic assessment. The compositions of the invention may be administered locally or systemically. The compositions of the invention may also be administered directly to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents depending on the intended use of the pharmaceutical composition.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use. After pharmaceutical compositions have been prepared, they can be placed in an appropriate container and labeled for treatment of an indicated condition. Pharmaceutical compositions suitable for use in the invention include compositions wherein the active ingredients are contained in an effective amount to achieve the intended purpose. The determination of an effective dose is well within the capability of those skilled in the art. For any compounds, the therapeutically effective does can be estimated initially either in cell culture assays, e.g., of cultured neuronal cells, cell lines, or in animal models, usually mice, rabbits, dogs, or pigs. The animal model may also be used to determine the appropriate concentration range and route of administration. Such information can then be used to determine useful doses and routes for administration in humans. A therapeutically effective dose refers to that amount of active ingredient, for example the activators, inhibitors, agonists or antagonsits of unc13/Munc13 or specific Munc13-isoforms. Therapeutic efficacy can toxicity may be determined by standard pharmaceutical procedures in cell cultures or experimental animals, e.g., ED50 (the does therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage from employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art employ different formulations for nucleotides than for proteins or their inhibitors.

Accordingly, the present invention also relates to a method for preventing, ameliorating and/or treating a neurological or a secretarial disorder or disease comprising the administration of Munc13 polypeptide or a fragment thereof or of a polynucleotide encoding a Munc13 polypeptide or a fragment thereof or of a molecule as identified by the method of the invention. Preferably, said Munc13 polypeptide is a specific isoform of Munc13. Most preferably, the individual to be treated is a human.

The invention also provides for the use of a transgenic, non-human animal for identifying and/or obtaining a molecule which is capable of modifying secretion processes and/or which is an isoform-specific modulator.

Preferably, said transgenic animal comprises in its somatic and/or germ cells at least one gene encoding Unc13 (or a part or a fragment thereof) and/or a Munc13-isoform (or a part or a fragment thereof). Such an animal may be a “knock-in” animal. Yet, said transgenic animal to be used may also be a transgenic animal which does not express a functional Unc13 (or a part or a fragment thereof), a functional Munc13-1 (or a part or a fragment thereof), a functional Munc13-2 (or a part or a fragment thereof), a functional Munc13-3 (or a part or a fragment thereof). Such animal may be a transgenic animal in which the corresponding gene has been inactivated or deleted (e.g. a “knock-out animal” or an animal expressing a mutated version of the corresponding Unc13/Munc13isoform). In said “knock-out animal” Unc13/Munc13-1 isoform(s) is/are inactivated or suppressed. The appended examples illustrate how such transgenic animals may be obtained and employed in the methods of the present invention. Preferred examples of such transgenic animals are Munc13-1 “knock-in” animals as well as Munc13-2 “knock-out” animals as described and characterized in the appended examples.

The figures show:

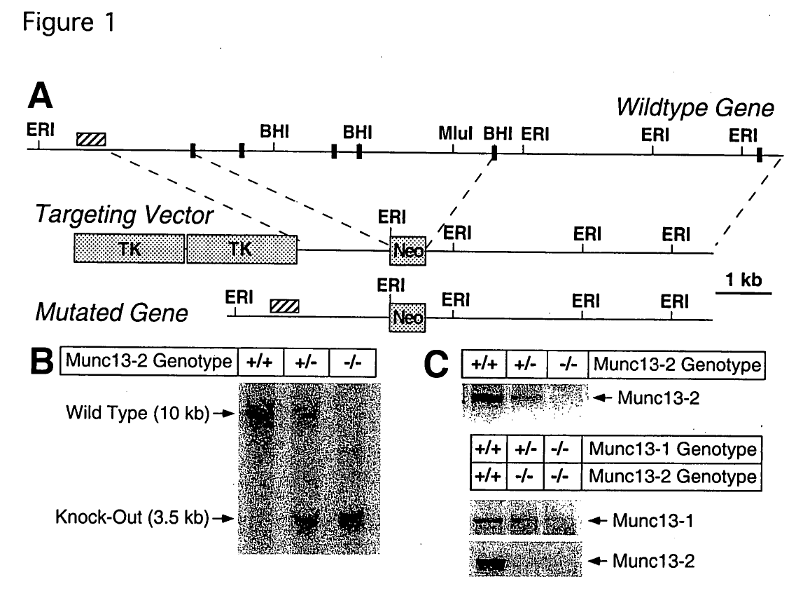

FIG. 1. Mutation of the Murine Munc13-2 Gene

(A) Deletion of Munc13-2 in the mouse. Maps of the wildtype Munc13-2 gene, the respective targeting vector, and the resulting mutant gene are shown. Positions of exons (black boxes) and restriction enzyme sites are indicated. The position of the probe used to identify the mutant allele is indicated by a hatched bar. Neo, neomycin resistance gene; TK, thymidine kinase gene.

(B) Southern blot analysis of the Munc13-2 deletion mutations in mice. Mouse tail DNA from adult wildtype mice (+/+), and mice heterozygous (+/−) or homozygous (−/−) for the mutation in the Munc13-2 gene were analyzed as described in Experimental Procedures. Positions of wild type and mutant (knock-out) alleles are indicated.

(C) Munc13-2 expression in deletion mutant mice. Brain homogenates (20 μg protein per lane) from adult wildtype (+/+), heterozygous (+/−) and homozygous (−/−) mutant mice were analyzed by SDS-PAGE and immunoblotting using an antibody that recognizes both, bMunc13-2 and ubMunc13-2.

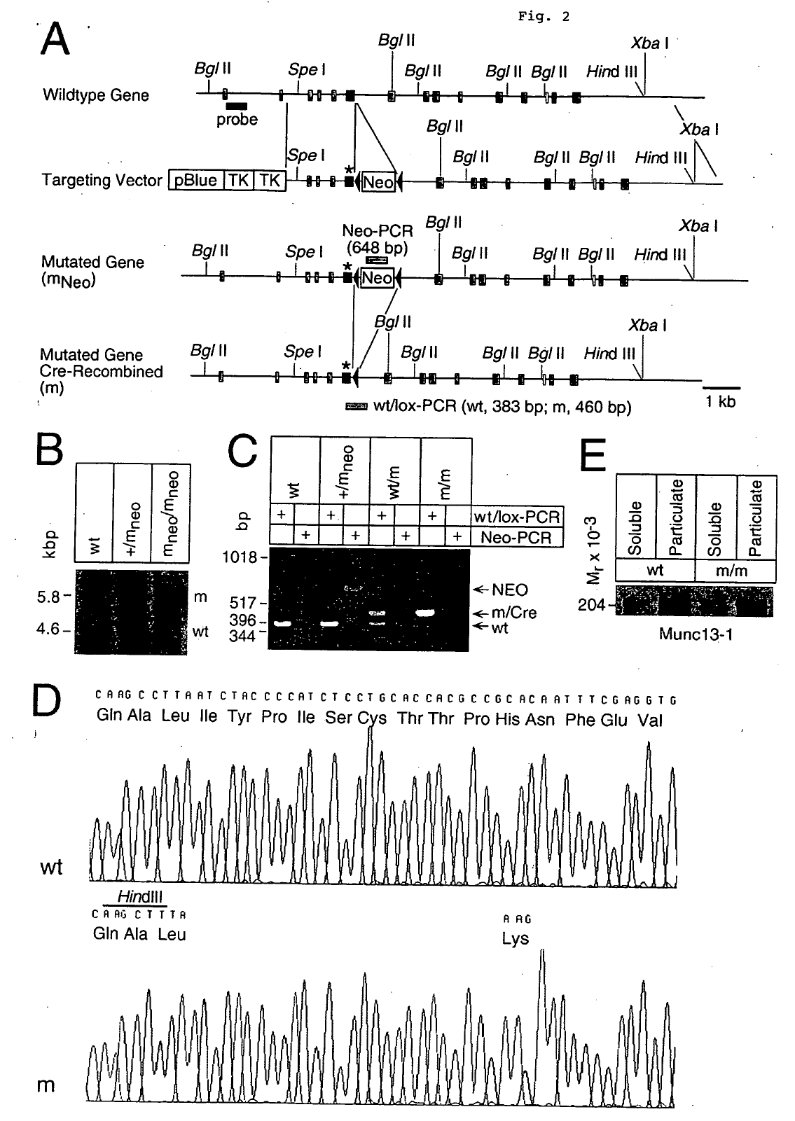

FIG. 2. Strategy for the Generation of the Munc13-1^{H567K} Mutation in Mouse Embryonic Stem Cells and Basic Characterization of Homozygous Mutant Mice

(A) Wild type Munc13-1 gene (wt), targeting vector, mutated gene after homolgous recombination (m_{Neo}), and mutated gene after Cre recombination (m). Exons are indicated by black (C₁ domain) or gray (all other) boxes. Black triangles indicate loxP sites. Asterisk indiates H567K mutation. Black horizontal bar indicates probe used for Southern analysis (Bgl II digested tail DNA) of mutated genes in mice. Products of diagnostic PCR reactions (size in brackets) are indicated by horizontal gray bars. Neo, neomycin resistance gene; pBlue, pBluescript KS; TK, herpes simplex virus thymidine kinase.

(B) Southern blot analysis of mutated genes using Bgl II digested mouse tail DNA and the probe indicated in (A). m_{Neo}, mutated gene; wt, wild type.

(C) PCR analysis of indicated phenotypes using the PCR strategy depicted in (A). m, mutated gene after Cre recombination; m_{Neo}, mutated gene; wt, wild type.

(D) Sequence analysis of PCR products obtained with the wt/lox PCR depicted in (A) and (C) using mouse tail DNA as template. wt, wild type; m, mutated gene after Cre recombination

(E) Western blot analysis of brain homogenates from wild type (wt) and Munc13-1^{H567K}/Munc13-1^{H567K} (m/m) mice. Soluble and particulate fractions were separated by centrifugation at 300.000×g for 10 min. Proteins were separated by SDS-PAGE and immunoblotted for Munc13-1 using a specific monoclonal antibody to the C-terminus of the protein.

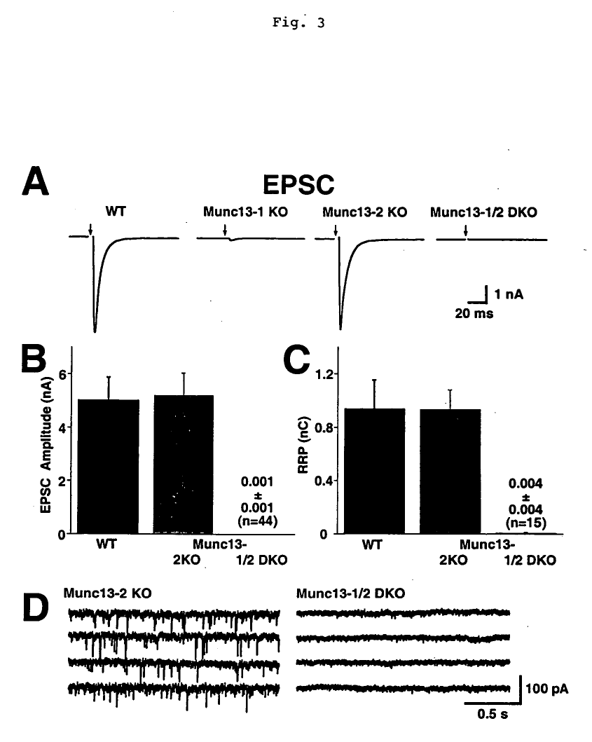

FIG. 3. Partial Redundancy of Munc13 Isoforms in Excitatory Neurons

(A) Examples of EPSCs recorded from wild type, Munc13-1, Munc13-2 and Munc13-1/2 deficient neurons.

(B) Mean EPSC amplitudes measured in wild type, Munc13-2, and Munc13-1/2 deficient neurons.

(C) Mean readily releasable vesicle pool sizes in wild type, Munc13-2, and Munc13-1/2 deficient neurons as estimated by the charge integral measured following release induced by application of 500 mOsm hypertonic solution for 4 seconds.

(D) mEPSC activity recorded from a Munc13-1/2 double deficient (left) and a Munc13-2 deficient neuron (right). Holding potential −70 mV.

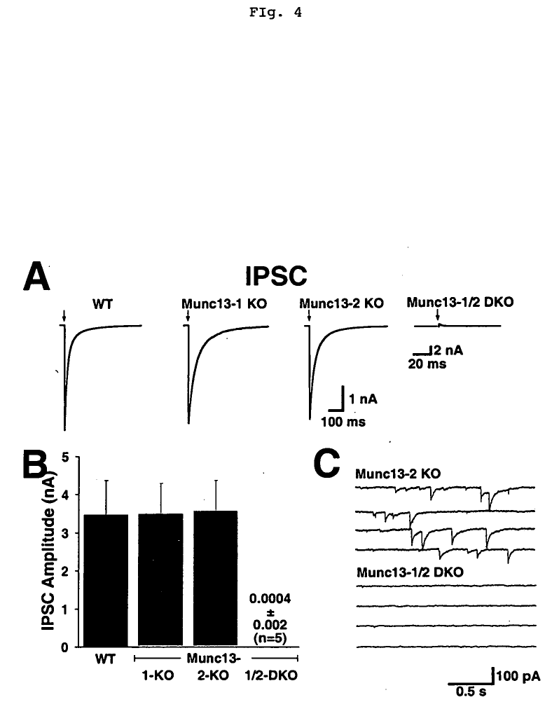

FIG. 4. Complete Redundancy of Munc13 Isoforms in Inhibitory Neurons

(A) Exampes of IPSCs recorded from wild type, Munc13-1, Munc13-2 and Munc13-1/2 deficient neurons. Munc13-1/2 deficient inhibitory neurons were identified following each experiment by detection of mIPSC events induced by application of α-latrotoxin.

(B) Mean IPSC amplitudes measured in wild type, Munc13-2, and Munc13-1/2 deficient neurons.

(C) mIPSC activity recorded from a Munc13-2 deficient (top) and a Munc13-112 double deficient neuron (bottom). Pipette solution contained 140 mM KCl.

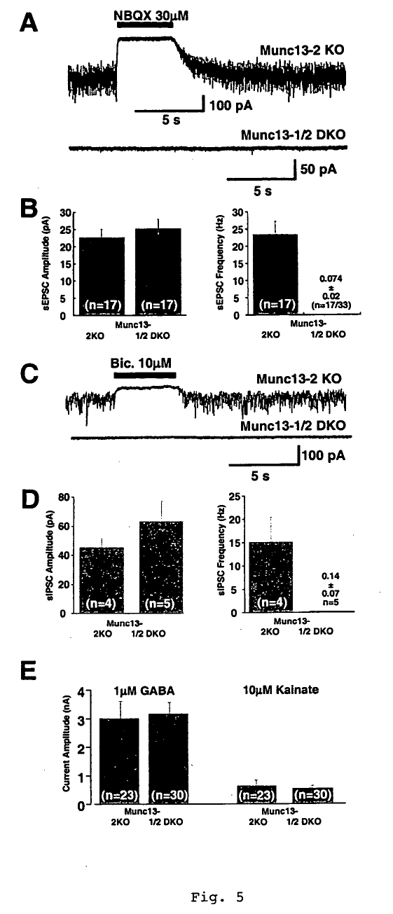

FIG. 5. Normal Postsynaptic Responsiveness in Release Incompetent Munc13-1/2 Double Deficient Neurons

(A) Examples of mEPSC activity from a Munc13-2 deficient control cell (top) and a Munc13-1/2 double deficient neuron (bottom) following application of 1 nM α-latrotoxin for 1 minute. Note that signals are blocked by NBQX.

(B) Average mEPSC amplitude and frequency following treatment of Munc13-2 deficient control and Munc13-1/2 double deficient neurons with 1 nM α-latrotoxin.

(C) Examples of mIPSC activity from a Munc13-2 deficient control cell (top) and a Munc13-1/2 double deficient neuron (bottom) following application of 1 nM α-latrotoxin for 1 minute. Note that signals are blocked by bicuculline.

(D) Average mIPSC amplitude and frequency following treatment of Munc13-2 deficient control and Munc13-1/2 double deficient neurons with 1 nM α-latrotoxin.

(E) Mean peak current responses evoked by exogenous application of GABA and kainate recorded from Munc13-2 deficient (black bars) and Munc13-1/2 double deficient neurons (white bars).

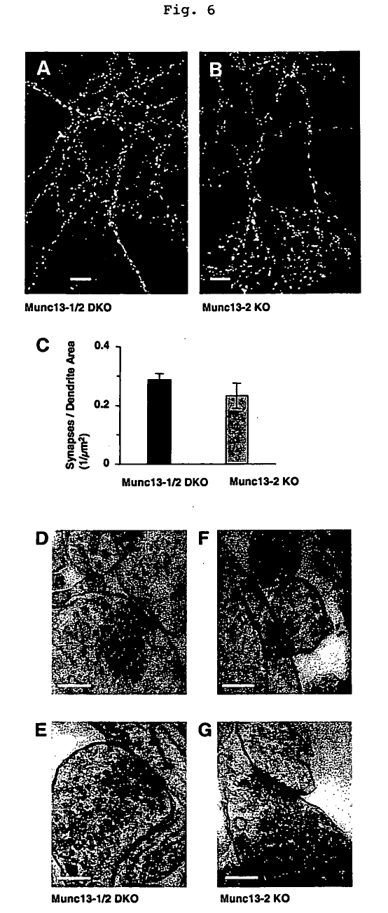

FIG. 6. Normal Synapse Density and Morphology in Release Incompetent Munc13-1/2 Double Deficient Neurons

(A, B) Staining of a Munc13-2 deficient (A) and a Munc13-1/2 double deficient (B) cell for the synapse specific marker Synaptophysin. A secondary antibody conjugated to Alexa-488 was used. Bar: 10 μm.

(C) Comparison of the synapse density, calculated from the number of identified synapses and the dendrite area (n=9 and 10 cells).

(D, E) Electron micrographs of an example of a Munc13-2 deficient (left) and a Munc13-1/2 double deficient synapse (F,G). Bars: 500 nm.

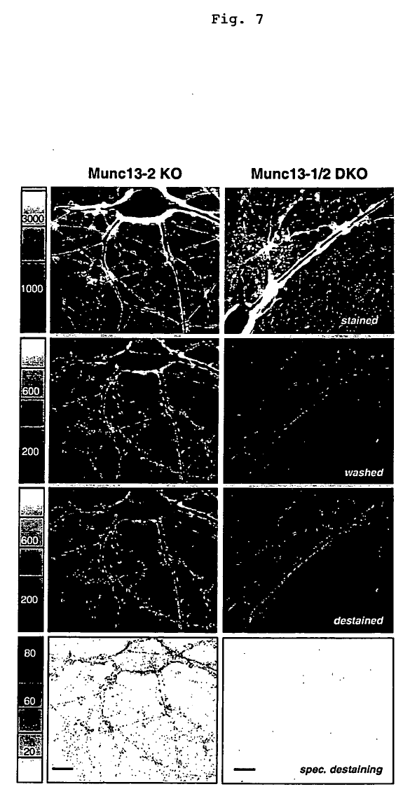

FIG. 7. No Endocytosis in Release Incompetent Munc13-1/2 Double Deficient Neurons

Localization of endocytotically and exocytotically active synapses by activity dependent FM1-43 staining and destaining in Munc13-2 deficient (left) and Munc13-1/2 double deficient (right) neurons. Top row, images during staining; second row, images after 3 minutes wash; third row, images following 20 s of destaining with K⁺; bottom row, specific loss of fluorescence as calculated from procedures described in Experimental Procedures. Gray scale bars on the left indicate fluorescence intensity. Bar: 10 μm.

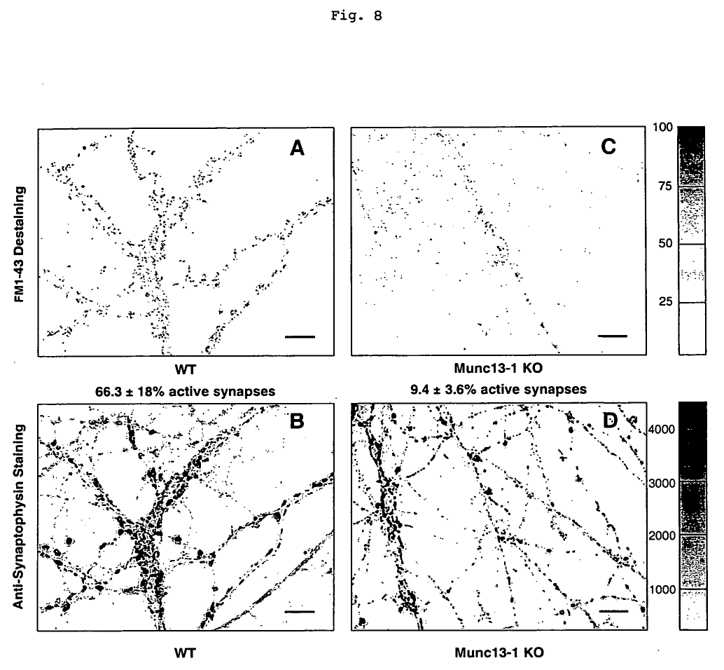

FIG. 8. Distinct Pools of Synapses Employing Munc13-1 or Munc13-2 as Priming Factors

(A, C) Localization of active synapses in dendritic regions of autaptic neurons. Difference images representing specific FM1-43 destaining (see Experimental Procedures) were calculated in wild type (A) and Munc13-1 deficient neurons (C).

(B, D) Localization of all synapses in the same cell regions as shown in (A) and (C). Neurons were stained with an antibody to Synaptophysin followed by a secondary Alexa-488 conjugated antibody.

(E) The fraction of active synapses determined for glutamatergic wild type (n=18 cells) and Munc13-1 deficient cells (n=12 cells). Ratios were calculated by dividing the number of synapses that stained with the antibody to Synaptophysin and stained/destained with FM1-43 by the total number of Synaptophysin positive synapses in the same region. Bar: 10 μm.

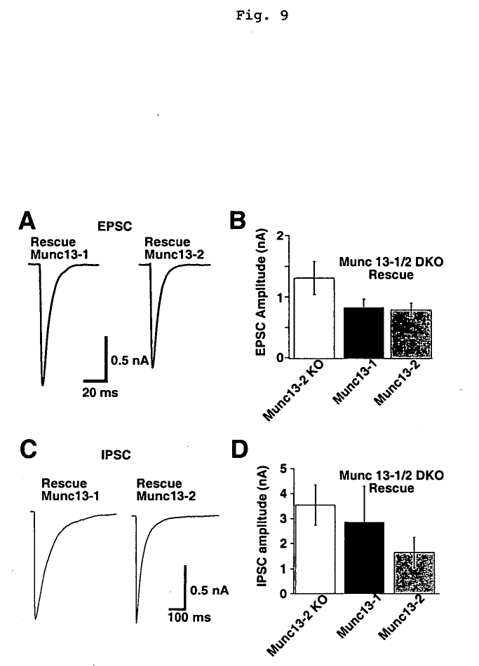

FIG. 9. Rescue of EPSCs and IPSCs in Munc13-1/2 Double Deficient Neurons By Virus Mediated Overexpression of Munc13-1 or ubMunc13-2

(A, C) Typical EPSC (A) and IPSC(C) from Munc13-1/2 double deficient neurons following virus mediated overexpression of Munc13-1 or Munc13-2.

(B, D) Mean EPSC (B) and IPSC (D) amplitudes from Munc13-2 deficient neurons (white bars), and from Munc13-1/2 double deficient neurons after virus mediated overexpression of Munc13-1 (black bar) or Munc13-2 (gray bar).

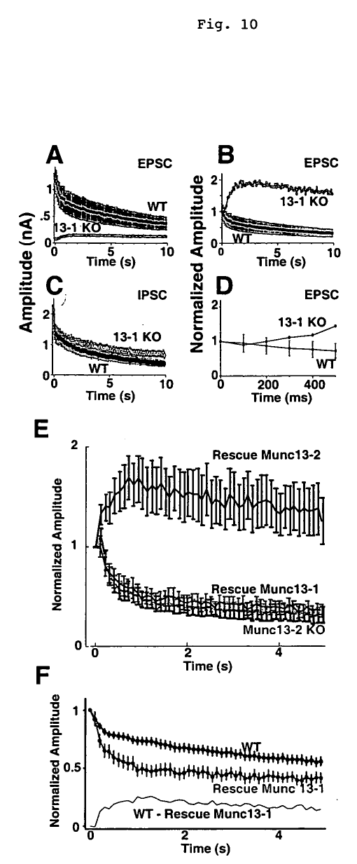

FIG. 10. Depression and Augmentation of EPSCs During Action Potential Trains in Munc13-1 and Munc13-2 Dependent Synapses

(A, B) Absolute (A) and normalized (B) synaptic responses from excitatory wild type and Munc13-1 deficient cells during a 10 Hz train (10 s). Wild type EPSCs (WT, black circles) show about 70% depression after 100 evoked responses. EPSCs in Munc13-1 deficient cells are much smaller than wild type signals but increase to 170% of the initial amplitude.

(C) Normalized IPSC amplitudes from wild type (WT, black circles) and Munc13-1 deficient cells (white circles). IPSCs in both genotypes show about 70% depression after 100 evoked responses.

(D) Expanded time course of data shown in (B) to illustrate the delayed increase of EPSC amplitudes in Munc13-1 deficient cells beginning with the fourth EPSC.

(E) Normalized EPSC amplitudes during a 10 Hz train measured in Munc13-2 deficient, wild type like control neurons and in Munc13-1/Munc13-2 double deficient cells overexpressing either Munc13-1 or ubMunc13-2.

(F) Comparison of amplitude time courses during 10 Hz train in wild type (black circles, n=21) and Munc13-1/Munc13-2 double deficient cells overexpressing Munc13-1 (gray circles, n=19). The black line is the calculated difference between the two groups and resembles the amplitude time course of the Munc13-2 dependent synapses (see FIGS. 8B and 8E). Note that the data sets of FIGS. 1A-1D and FIGS. 1E-1F were obtained in separate experiments.

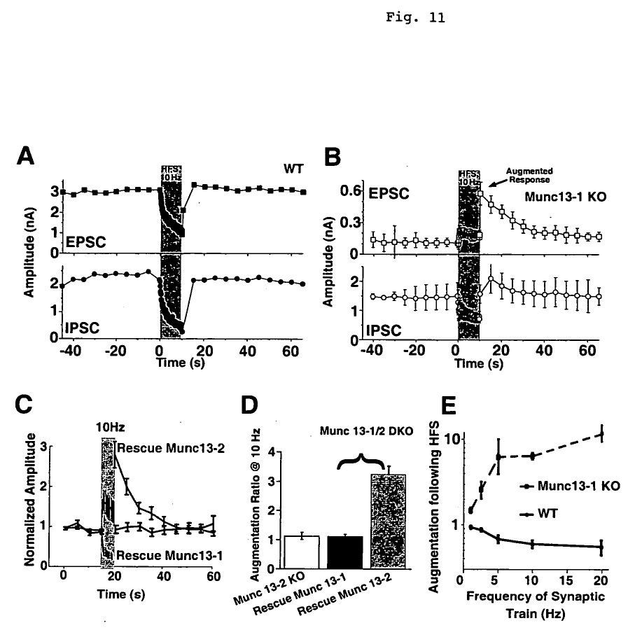

FIG. 11. EPSC Amplitudes in Munc13-1 Deficient, Munc13-2 Dependent Synapses are Augmented Following Action Potential Trains

(A, B), Average absolute EPSCs (top) and IPSCs (bottom) before, during (gray area), and after a 10 Hz train (50 stimuli) in wild type (A) and Munc13-1 deficient cells. To determine augmentation ratios, augmentation of the EPSC was measured 2 s after the train.

(C) Normalized EPSCs showing depression and augmentation following a 10 Hz action potential train (50 stimuli) in Munc13-1/Munc13-2 double deficient neurons overexpressing Munc13-1 (bottom trace) or Munc13-2 (top trace), respectively.

(D) Average augmentation in Munc13-2 deficient, Munc13-1 dependent, wild type like control neurons (white bar), and in Munc13-1/Munc13-2 double deficient neurons overexpressing Munc13-1 (black bar) or ubMunc13-2 (gray bar). Give n values

(E) Average augmentation of EPSCs in wild type (black squares) and Munc13-1 deficient cells (black circles) as a function of the train frequency (1 Hz, 20 stimuli; 2.5 Hz, 40 stimuli; 5 and 10 Hz, 50 stimuli; 20 Hz, 100 stimuli). Measurements are from 17-45 cells.

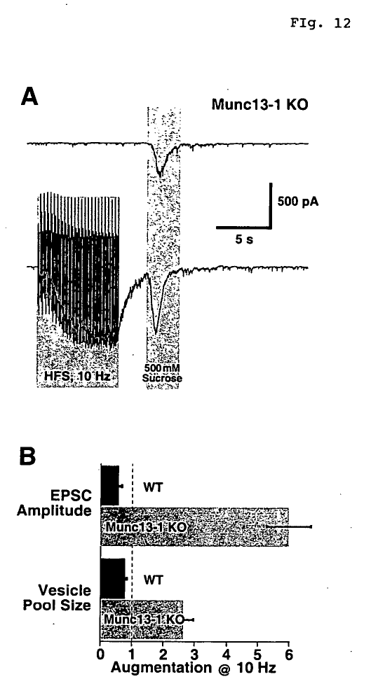

FIG. 12. Augmentation is Accompanied by an Increase of the Readily Releasable Vesicle Pool and of the Vesicular Release Probability

(A) Two current responses to hypertonic sucrose stimulation (3 s, 500 mOsm added to the external medium, gray area) from the same excitatory Munc13-1 deficient neuron before (top) and immediately after (bottom) a 10 Hz action potential train.

(B) Bar graph showing the average synaptic augmentation and pool size change following the 10 Hz train for wild type (black bars) and Munc13-1 deficient neurons (gray bars). Data were normalized to the control EPSC/pool size measurement. Dotted line indicates the size of the control EPSC/pool size.

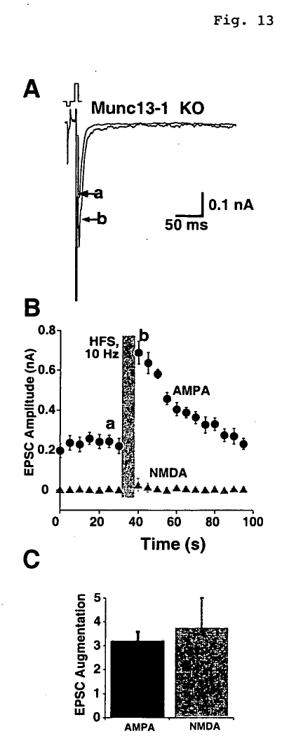

FIG. 13. Augmentation is Not Due to a Reawakening of Silent Synapses Munc13-1 deficient neurons. Synaptic NMDA EPSCs were initially blocked by a series of EPSCs stimulated at low stimulation frequency (0.2 Hz) in the presence of 5 pMMK-801.

(A) Dual component EPSCs after washout of MK-801 at time points immediately preceding (a) or following (b) the 10 Hz train (7 s) are shown for an example neuron. The remaining slow NMDA component of the EPSC augmented not more than expected.

(B) Time course of averaged AMPA and NMDA mediated amplitudes before and after the 10 Hz train (gray area).

(C) Average augmentation of the remaining NMDA (gray bar) and the AMPA component (black bar) 2 seconds following the 10 Hz train compared to the control period preceding the train (n=6).

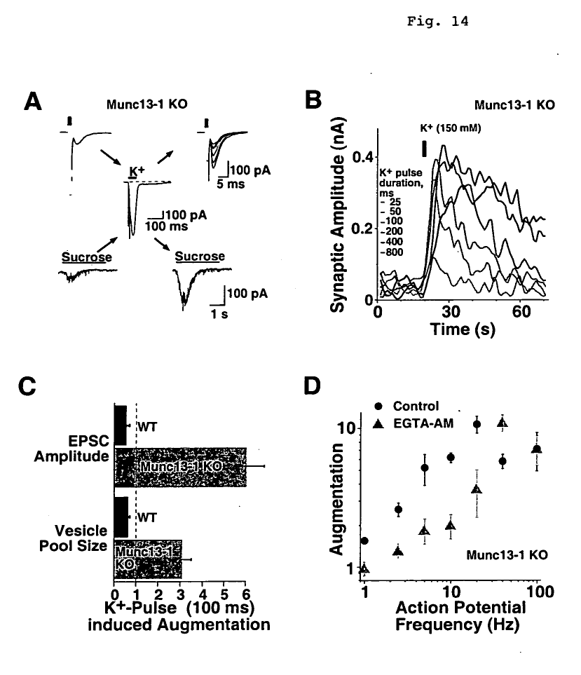

FIG. 14. Augmentation Depends on Increases of the Intracellular Ca²⁺ Concentration

(A) Synaptic (upper row) and sucrose induced (lower row) EPSC responses from a Munc13-1 deficient neuron before (left site) and after (right site) a 100 ms application of 150 mM KCl (in standard external solution) to the entire neuron (middle). The KCl application induced a somatic inward current, presumably due to the opening of inward rectifier K⁺ channels, and was usually accompanied by several unclamped spikes. The tail current following the K⁺ pulse was reminiscent of massive mEPSC activity due to activation of voltage dependent Ca²⁺ channels in the unclamped nerve terminal population. The right site of the panel shows the transient augmentation of the synaptic response seen in the series of EPSCs recorded at 0.2 Hz (top right) and the increase of the vesicle pool two seconds following the K⁺-pulse (bottom right).

(B) Augmentation of EPSC amplitudes (measured at 1 Hz) in a Munc13-1 deficient neuron induced by K⁺ pulses of increasing duration (150 mM; 25, 50, 100, 200, 400, 800 ms; from light gray to black traces). Augmentation was maximal with a 100 ms pulse. Longer pulses led to delayed (due to pool depletion) and longer lasting augmentations. Similar observations were made in 5 additional cells.

(C) Bar plot showing the degree of modulation of the synaptic response and pool size in wild type and Munc13-1 deficient glutamatergic neurons by a 100 ms pulse (150 mM, 4 mM external Ca²⁺).

(D) Increased presynaptic Ca²⁺ buffering reduces frequency dependent augmentation. Action potential frequency/augmentation profile from control (black circles) and EGTA-AM treated (gray triangles) Munc13-1 deficient neurons. Augmentation in EGTA-AM treated cells was consistently lower for frequencies of 1-20 Hz. Numbers of stimuli were as given in FIG. 2E; 40 Hz, 100 stimuli; 100 Hz, 100 stimuli. Measurements are from 5-12 cells. Control data set is identical to FIG. 13C.

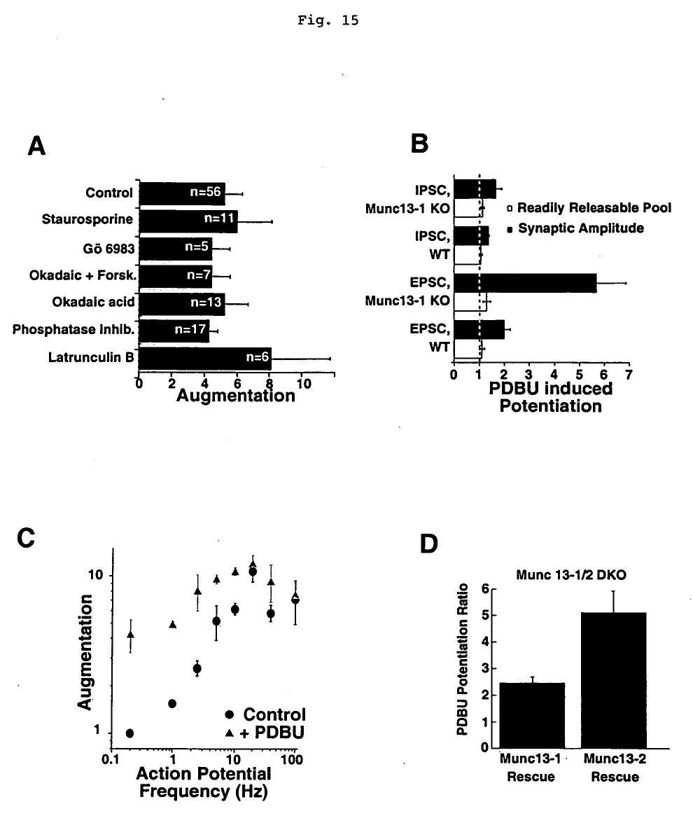

FIG. 15. Augmentation depends on Phospholipase C activity and is Mimicked by β-Phorbol Esters But is Independent of Kinases, Phosphatases or Changes in Actin Cytoskeletal Dynamics

(A) Bar diagram showing the degree of EPSC augmentation in Munc13-1 deficient cells in the absence (control) or presence of various drugs. Blockers of kinases and phosphatases (cypermethrin, 1 nM; calyculinA, 10 μM; cyclosporine A, 1 μM), and blockers of actin polymerization (latrunculin B; 10 μM) were ineffective in blocking augmentation. Likewise, a combination of forskolin (1 μM) with the phosphatase 2A inhibitor okadaic acid (1 μM) or okadaic acid alone (1 μM) were unable to block augmentation. However, treatment with the phospholipase C inhibitor U73222 (3 μM), but not its inactive analogue U73445 (3 μM) largely inhibited the induction of augmentation.

(B) Effects of the β-phorbol ester PDBU (1 μM, 60 s) on synaptic (wt, n=9; KO, n=17) and sucrose (wt, n=8; KO, n=11) induced IPSC and synaptic (wt, n=7; KO, n=17) and sucrose (wt, n=4; KO, n=5) induced EPSC responses in wild type and Munc13-1 deficient neurons. Synaptic and sucrose induced responses were normalized to control responses measured before PDBU application. Dashed line indicates the control value.

(C) Nonadditivity of action potential train induced augmentation and β-phorbol ester induced potentiation in Munc13-1 deficient neurons. Action potential frequency/augmentation profile from Munc13-1 deficient neurons in the absence (Control, black circles) or presence of PDBU (1 μM, gray triangles). Data for PDBU treated cells were normalized to the basal EPSC amplitude measured before treatment. EPSC augmentation in untreated and treated synapses reached similar values for frequencies at and above 20 Hz. Numbers of stimuli were as given in FIG. 12D. Measurements are from 4-12 cells.

(D) β-Phorbol ester induced EPSC potentiation in Munc13-1/Munc13-2 double deficient neurons that had been rescued by viral overexpression of Munc13-1 (n=11) or Munc13-2 (n=12).

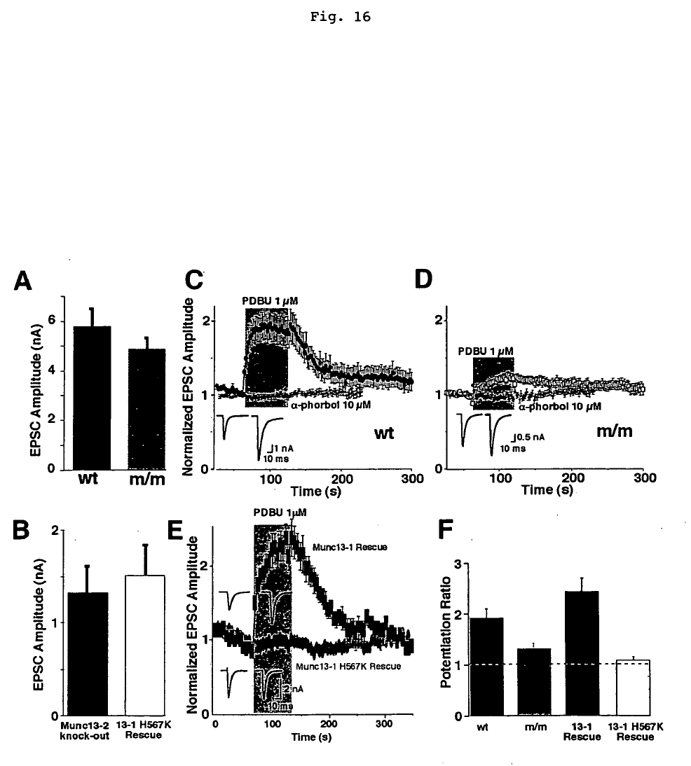

FIG. 16. Effects of the Munc13-1^{H567K} Mutation on Synaptic Responses and Their A-PE Mediated Potentiation

(A) Evoked excitatory autaptic currents from wild type (n=83) and Munc13-1^{H567K}/Munc13-1^{H567K} (n=91) neurons. Error bars indicate standard error of mean.

(B) Synaptic currents from Munc13-2 deficient (n=17) and Munc13-1/Munc13-2 double deficient cells overexpressing Munc13-1^{H567K} (n=21). Error bars indicate standard error of mean.

(C, D) Time course of β-PE effects on evoked EPSCs in wild type (C; n=30) and Munc13-1^{H567K}/Munc13-1^{H567K} (D; n=34) neurons. Application period (1 min) of β-PE dibutyrat (PDBU, 1 μM) or its inactive analogue α-phorbol (10 μM, assayed in separate experiments) is indicated by a gray box. EPSCs were evoked at 0.5 Hz and normalized to the initial amplitude. Error bars indicate standard error of mean.

(E) Time course of β-PE effects on evoked EPSCs in Munc13-1/Munc13-2 double deficient cells overexpressing wild type Munc13-1 (n=12) or Munc13-1^{H567K} (n=17). Application period (1 min) of β-PE dibutyrat (PDBU, 1 μM) is indicated by a gray box. EPSCs were evoked at 0.5 Hz and normalized to the initial amplitude. Error bars indicate standard error of mean.

(F) Average potentiation of evoked EPSC amplitude from experiments shown in (E) 30 s following onset of PDBU application. Dotted line is control amplitude. Holding potential—75 mV. Error bars indicate standard error of mean.

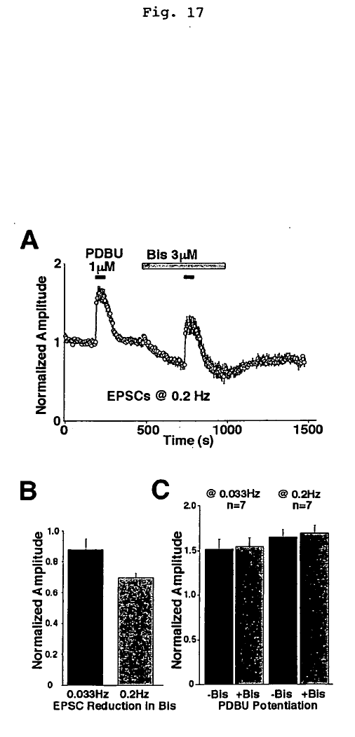

FIG. 17. Pharmacology of β-PE Induced Potentiation in Wild Type Neurons

(A) Time course of β-PE effects on evoked EPSCs in wild type neurons (n=6). Application of PDBU (1 μM) is indicated by a white box.

(B) Effects of bisindolylmaleimide 1 (3 μM; gray box) on the same neurons as recorded in (A). Preincubation of bisindoylmaleimide I led to a partially irreversible rundown of evoked EPSC amplitudes, but did not block the potentiation induced by application of PDBU (1 μM) for 1 min (white box).

(C) Average evoked EPSC amplitudes before β-PE application in untreated neurons before and after bisindoylmaleimide 1 (3 μM) pretreatment (n=6).

(D) Average β-PE mediated potentiation in untreated and bisindoylmaleimide I (3 μM) pretreated neurons, calculated by dividing the evoked EPSC amplitude 30 seconds following by that immediately before application of 1 μM PDBU (n=6).

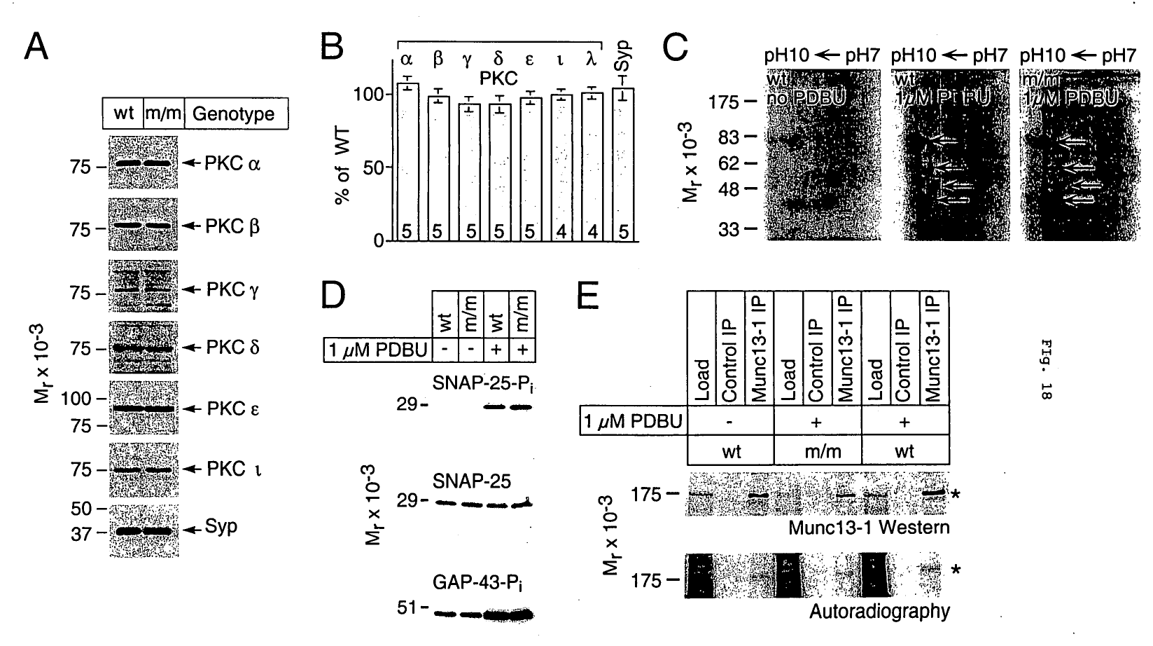

FIG. 18. Expression and Function of PKCs in Munc13-1^{H567K}/Munc13-1^{H567K} Neurons

(A) Western blot analyses of various PKCs and Synaptophysin (Syp) in brain homogenates from a wild type (wt) and a Munc13-1^{H567K}/Munc13-1^{H567K} (m/m) brain.

(B) Quantitative analysis of PKC levels determined by Western blotting. Protein levels in Munc13-1^{H567K}/Munc13-1^{H567K} brains are expressed as % of wild type controls. Respective n values are given at the base of histogram bars. Syp, synaptophysin. Error bars indicate standard error of mean.

(C) Autoradiographs of 2D gels (selected sections) obtained from ³²P-labeled cultures of wild type (wt) and Munc13-1^{H567K}/Munc13-1^{H567K} (m/m) brains in the absence or presence of β-PE (1 μM PDBU). Arrows indicate proteins that are specifically phosphorylated in a β-PE dependent manner.

(D) β-PE dependent phosphorylation of SNAP-25 and GAP-43 in wild type (wt) and Munc13-1^{H567K}/Munc13-1^{H567K} (m/m) cultures. Cultures were treated as decribed in Experimental Procedures. Identical amounts of proteins were separated by SDS-PAGE and probed with specific antibodies for phosphorylated SNAP-25 (SNAP-25-P_{I}), total SNAP-25 (SNAP-25), and phosphorylated GAP-43 (GAP-43-P_{I}).

(E) Test for β-PE dependent phosphorylation of Munc13-1. Wild type (wt) and Munc13-1^{H567K}/Munc13-1^{H567K} (m/m) cultures were labeled with ³²P-orthophosphate and stimulated with 1 μM PDBU. Proteins were extracted and Munc13-1 was immunoprecipitated using specific polyclonal antibodies (Munc13-1 IP) or control preimmune serum (Control IP). Immunoprecipitated material as well as the extract before precipitation (Load) were separated by SDS-PAGE. Labeled proteins were visualized by autoradiography. Immunoprecipitated Munc13-1 was detected by immunoblotting with a specific monoclonal antibody. Asterisks indicate Munc13-1 bands.

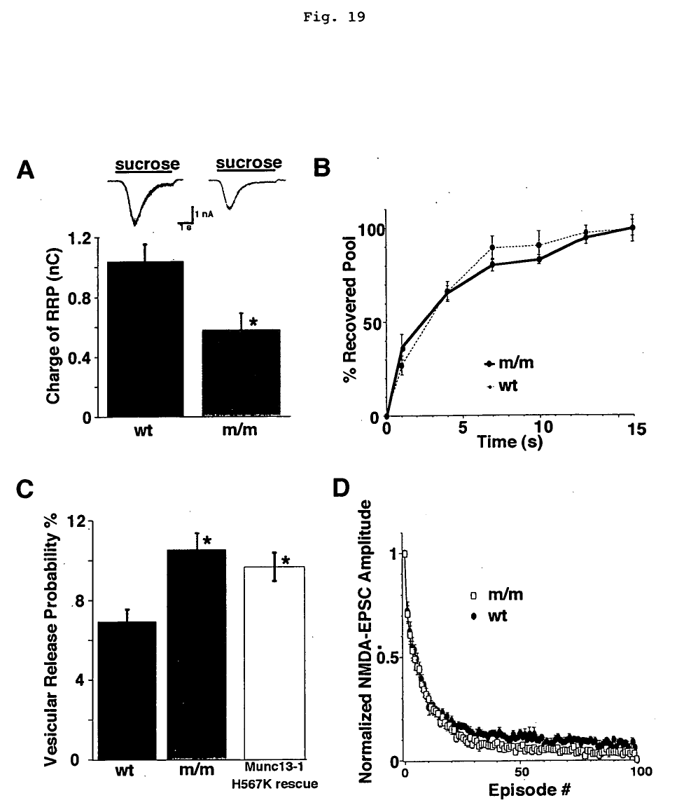

FIG. 19. Readily Releasable Vesicle Pool and Release Probability in Wild Type and Munc13-1^{H567K}/Munc13-1^{H567K} Neurons

(A) Average transient charge component of release of the readily releasable vesicle pool in glutamatergic neurons induced by hypertonic sucrose solution (500 mOsm hypertonic, 4 seconds). Asterisk indicates significant reduction (p<0.001) of the readily releasable vesicle pool in Munc13-1^{H567K}/Munc13-1^{H567K} (n=81) over wild type control neurons (n=72).

(B) Refilling kinetics of the readily releasable vesicle pool under resting conditions as measured in a paired pulse protocol consisting of two 4 s applications of hypertonic solution at varying inter pulse intervals. The transient charge component of the second response was divided by the first response.

(C) Average vesicular release probability Pᵥᵣ of wild type (n=72) and Munc13-1^{H567K}/Munc13-1^{H567K} neurons (n=81) as well as Munc13-1/Munc13-2 double deficient neurons overexpressing Munc13-1^{H567K} (rescue; n=17) was calculated by dividing the evoked EPSC charge by the charge of the response following depletion of the readily releasable vesicle pool with hypertonic solution. Asterisks indicate significant increase (p<0.01) of Pᵥᵣ over wild type control neurons.

(D) Synaptic release probability as determined by NMDA-EPSC amplitude decay during ongoing EPSC stimulation in the presence of the irreversible NMDA open channel blocker MK-801 (5 μM). EPSC amplitude was normalized to the first response in the repsence of MK-801. External solution contained 2.7 mM Ca²⁺, no Mg²⁺, and 10 μM glycine. Stimulation frequency was 0.33 Hz.

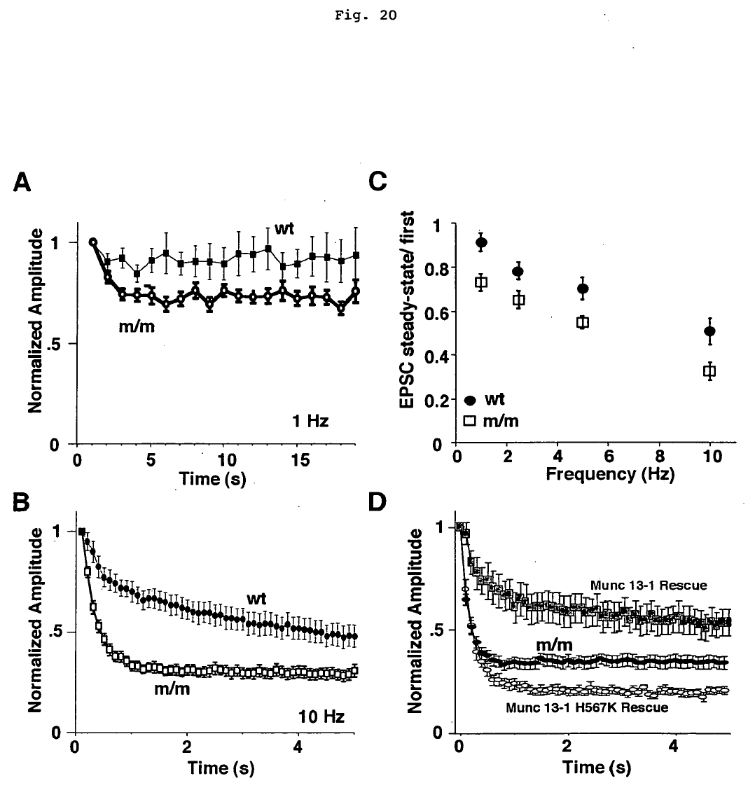

FIG. 20. Release Dynamics in Wild Type and Munc13-1^{H567K}/Munc13-1^{H567K} neurons

(A, B) Normalized evoked EPSC amplitudes of wild type (n=8) and Munc13-1^{H567K}/Munc13-1^{H567K} neurons (n=16) at 1 Hz (A) and 10 Hz (B) stimulation rates.

(C) Depression of EPSC amplitudes during action potential trains of 1, 2.5, 5 and 10 Hz as calculated from steady-state amplitude divided by first amplitude of the train (n=6-8 for wild type and n=15-16 for Munc13-1^{H567K}/Munc13-1^{H567K} neurons).

(D) Comparison of 10 Hz depression time course between Munc13-1^{H567K}/Munc13-1^{H567K} neurons (black circles; n=19) and Munc13-1/Munc13-2 double deficient neurons overexpressing wildtype Munc13-1 (gray squares; n=13) or Munc13-1^{H567K} (open circles; n=19). Note that depression is less pronounced in the Munc13-1^{H567K}/Munc13-1^{H567K} neurons presumably because of a remaining fraction of synapses that employ Munc13-2.

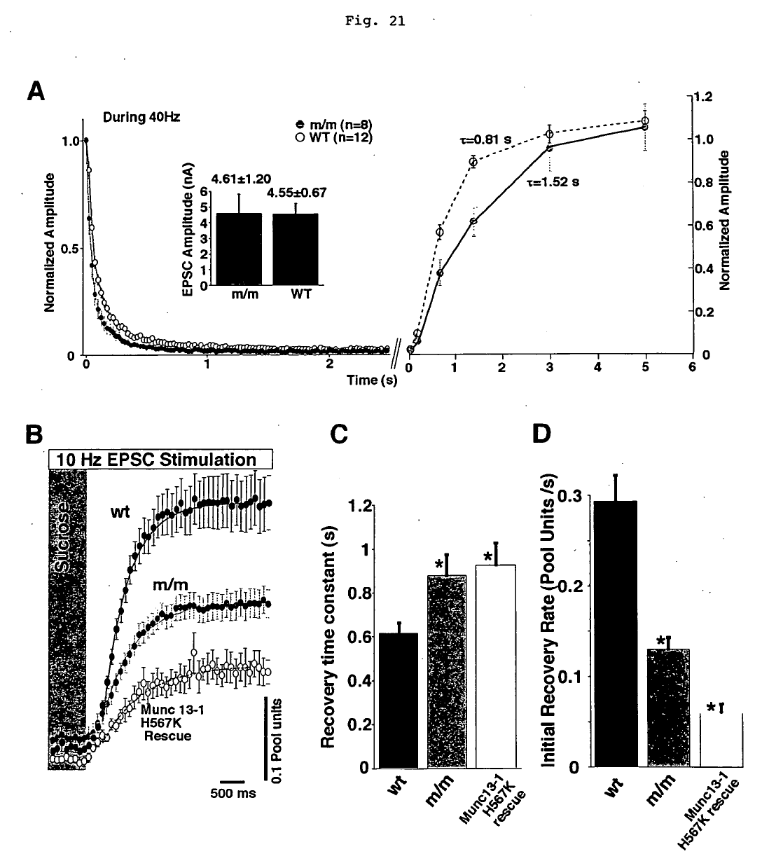

FIG. 21. Activity Dependent Refilling of Readily Releasable Vesicle Pools in Munc13-1^{H567K}/Munc13-1^{H567K} neurons.

(A) Recovery of evoked EPSC amplitudes following depletion of the pool by hypertonic sucrose application during ongoing 10 Hz stimulation. A train of 10 Hz was applied to excitatory cells. After 5 s of stimulation at 10 Hz (not shown), the readily releasable vesicle pool was intermittendly depleted by an application of hypertonic solution for 4 s (gray bar), and the recovery of the synaptic response during ongoing 10 Hz stimulation was monitored for another 10-20 s. Shown are average evoked EPSC amplitudes of wild type (black circles; n=29) and Munc13-1^{H567K}/Munc13-1^{H567K} cells (gray circles; n=36), as well as of Munc13-1/Munc13-2 double deficient neurons overexpressing Munc13-1^{H567K} (open circles; n=14) as they recover from depletion upon return to normal osmolarity of. The amplitudes were normalized to the initial EPSC amplitude in the train. It was assumed that the initial amplitude results from a fully filled readily releasable vesicle pool (1 pool unit).

(B) Time constants of recovery of evoked EPSC amplitudes following removal of hypertonic solution, calculated by an exponential fit of data set in (E). Values of n as in (E). Asterisks indicate significant change compared to wild type cells (p=0.015). Note that the recovery time constant is also influenced by the rate of depletion.

(C) Initial slope of recovery of evoked EPSC amplitudes following return to normal osmolarity, calculated from data set shown in (E). Values of n as in (E). Asterisks indicate significant change compared to wild type cells (p=0.015).

FIG. 22: A Calmodulin recognition motif_is present in UNC/Munc13 family members.

Upper panel: Predominantly Ca2+ dependent Calmodulin recognition motifs fall into several classes based on the position of hydrophobic residues within amphipathic helices. There are three known motifs (1-8-14 Type A, 1-8-14 Type B, 1-5-10). Amino acid residues that should occur at a given position are shown in brackets, while x can be any residue. Lower panel: Alignment of putative Calmodulin binding N-terminal fragments of several UNC/Munc13 proteins. These fragments are predicted to constitute an amphipathic helix with secondary structure prediction software and have a net positive charge. Hydrophobic residues occur at the following positions (W is at position one): 1-8-12. Given the flexibility of Calmodulin in recognizing its targets, these fragments constitute Calmodulin binding sites.

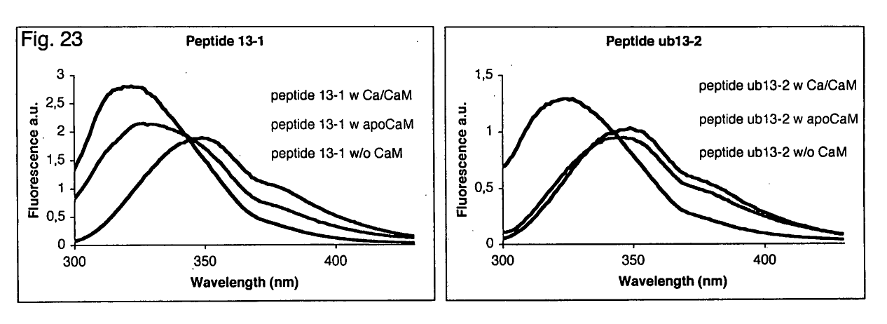

FIG. 23: Tryptophane fluorescence emission spectra of peptides reflecting the Calmodulin binding site of rat Munc13-1 and ubMunc13-2. 10 μM of each peptide were measured in NH4Acetate 0.25 M, 1 mM EGTA. Excitation was at 290 nm.

The Excitation shutter was adjusted to 1 nm bandwith, the emission shutter to 8 nm bandwidth. Pathlenght was 3 mm. All measurements were carried out at room temperature. The exact sequences of each peptide are given in the text. Upon addition of 10 μM apocalmodulin (Ca-free calmodulin) a blueshift is only observed for the maximum of peptide 13-1 but not ubMunc13-2, indicating partially Ca2+ independent binding for the Munc13-1 peptide. Both peptides show a pronounced blueshift and intensity increase upon addition of Ca2+/Calmodulin, indicating that binding is tighter in presence of Ca2+. In case of the Munc13-1 peptide, Calcium independent binding is also observed, while the binding of Munc13-2 peptides strictly requires Ca2+ for binding.

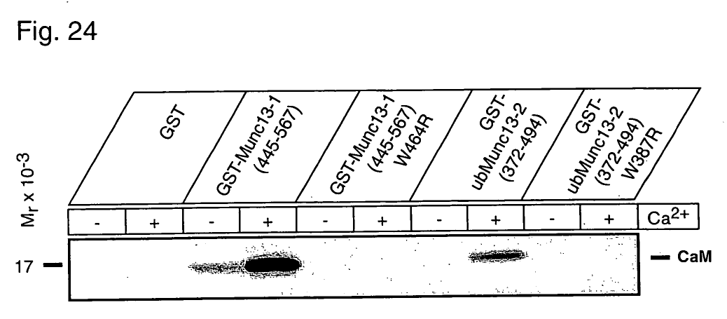

FIG. 24: Introduction of W/R point mutations into the Calmodulin binding sites of Munc13 proteins abolishes Calmodulin binding in cosedimentation assays.

GST fusion proteins containing the Calmodulin binding sites of rat Munc13-1 and ubMunc13-2 as well as GST alone were expressed in bacteria, purified and adsorbed onto glutathione beads in equal amounts for subsequent use in cosedimentation assays with rat brain synaptosome extract. Proteins that bound to the immobilized GST fusion proteins were analyzed by SDS-PAGE and immunoblotting with a specific Calmodulin antibody. Calmodulin binds specifically to wt GST-Munc13-1 (445-567) and GST-ubMunc13-2 (372-494). Calmodulin binding is completely abolished by the point mutations in the conserved tryptophanes (Position 1 in the alignment figure x). Furthermore it is shown that Calmodulin binding to the Munc13-1 fragment is partially independent of Ca²⁺.

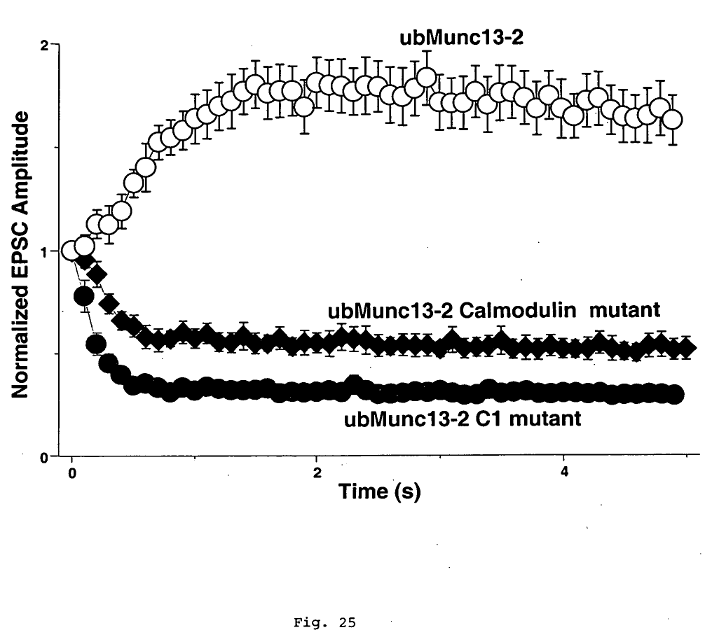

FIG. 25: The role of Calmodulin binding and DAG-binding to ubMunc13-2 for short-term plasticity.

Munc13-1/Munc13-2 double deficient neurons were either rescued by overexpression of ubMunc13-2 (n=20), or loss of binding point mutant derivatives at the calmodulin binding site (W387R, n=12) or DAG-binding. C region (H491K, n=16). In both cases, activity dependent maintenance of synaptic responses during trains of action potentials at 10 Hz were strongly blocked.

The examples illustrate the invention.

### EXAMPLE 1

### Experimental Procedures for the Following Illustrative Examples

A) Stem Cell Experiments and Mutant Mouse Strains

Munc13-2 deletion mutant mice were generated by homologous recombination in embryonic stem cells (Thomas, 1987; Augustin, 1999b; Reim, 2001). For that purpose, a targeting vector was generated on the basis of mouse genomic clone pM13-17.3 (FIG. 1A). In the targeting vector, a 6 kb genomic fragment containing multiple exons of the Munc13-2 gene which are shared by the two Munc13-2 splice variants (corresponding to bp 2907-3904 of bMunc13-2 cDNA [GenBank Acc. No. U24071] and bp 1439-2436 of ubMunc13-2 cDNA [GenBank Acc. No. AF159706]) was replaced by a neomycin resistance cassette. To allow negative selection of random integrations, the vector also contained two copies of the HSV thymidine kinase gene. Following electroporation and selection, recombinant stem cell clones were analyzed by Southern blotting after digestion of DNA with EcoR I. For hybridization, an outsite probe localized 5′ of the targeting vector short arm was used. Three positive clones were identified and two of these were injected into mouse blastocysts to obtain highly chimeric mice that transmitted the mutation through the germ line. Germ line transmission of the mutations was confirmed by Southern blotting of genomic DNA digested with EcoR I (FIG. 1B) and immunoblotting of brain extracts using an antibody directed against the C-terminus of Munc13-2 that is shared by both splice variants (FIG. 1C). Subsequently, routine genotyping was performed by PCR. Furthermore, the Munc13-1 deletion mutant as described in Augustin (1999) was employed.

B) Cell Culture and Electrophysiology

Microisland culture preparation was performed according to Bekkers and Stevens (1991). The extracellular medium contained (mM): NaCl, 167; KCl, 2.4; HEPES, 10; glucose, 10; CaCl₂, 4; MgCl₂, 4 (340 mOsm, pH 7.3). Solutions were applied and recordings were performed as described in Rosenmund (1995). To examine synaptically activated currents, experiments were performed using recurrent synapses (autapses) or only single autaptic neuroeses on microisland cultures at room temperature (=24° C.). Pipette solutions included (mM): KCl, 120, or K-Gluconate, 125; HEPES, 10; EGTA, 1; MgCl2, 4.6; Na₄ATP, 4; Creatinephosphate, 15; creatinephosphokinase 50 U/ml (320 mOsm, pH 7.3). Wild type and Munc13-1 deficient glutamatergic hippocampal neurons were used to study the characteristics of Munc13-1 and Munc13-1 independent synapses. For these purposes, individual neurons grown on microisland beds of glial cells (Bekkers, 1991) were employed. In this autaptic culture system, where isolated nerve cells make multiple synaptic contacts, so called autapses, with their own dendritic trees, spontaneous transmitter release, release of the readily releasable vesicle pool induced by hypertonic solutions, and action potential evoked release arise from the same synapse population and can be measured readily with patch clamp techniques. Autaptic responses were obtained after brief (1-2 ms) somatic depolarization. This induces an unclamped action potential that is followed by a postsynaptic response with a delay of 2-4 ms. Excitatory, glutamatergic and inhibitory, GABAergic synaptic currents were distinguished by their characteristic pharmacological and kinetic properties. Excitatory glutamatergic cells were mainly characterized because the differential equipment of presynaptic terminals formed by the same axon with Munc13 priming factors is not apparent in GABAergic neurons.

Data are expressed as mean±SE. Significance was tested using Student's t-test or one-way ANOVA with the Bonferroni-Dunn procedure for multiple comparisons (Instat).

Design of and infection with Semliki Forest Virus was done according to published procedures (Betz, 2001; Ashery, 2000). Cells were measured 22-26 h after infection.

C) Imaging Experiments

Hippocampal neurons were grown for 15-25 days on round glass cover slips (18 mm diameter, 0.17 mm thickness). Cover slips were used as the bottom glass of the perfusion chamber which was mounted on a stage of an inverted microscope (Olympus IX70) with an Olympus 40/1.35 or 60/1.4 NA oil objective. Fluorescence was excited at 488±7.5 nm using a Xenon lamp with a grating monochromator (Polychrome II, TILL Photonics). Emitted fluorescence light was bandpass filtered (525-575 nm) and detected with a CCD camera (1300×1030 pixel resolution; Princeton Micromax 1300YHS). Images were taken every 7 s with a 500 ms exposure time. To identify and count active synapses, cells were stimulated and synchronously stained with 15 μM FM1-43 (Molecular Probes) in two experiments per sample. In the first run, cells were stimulated by action potential induction (15 Hz, 10 s), in the second run by K⁺ induced depolarization (15 s; with modified external medium: 65 mM K⁺, 104.4 mM Na⁺). To release the endocytosed dye, neurons were stimulated again 180 s after staining by induction of action potentials (15 s, 20 Hz) or K⁺ mediated depolarization (20 s, 65 mM). All imaging experiments were performed at 30° C. The images were recorded using Axon Imaging Workbench 2.2 (Axon Instruments) and analyzed with TILLvisION 3.3 (TILL Photonics). For each series of images, the amount of specific destaining upon stimulation of the neuron for each pixel was calculated The amount of specific destaining (due to evoked exocytosis of stained vesicle content) was calculated from the fluorescence intensities in three images, which were recorded at 21 s time intervals: The first and the second image were recorded before stimulation/unloading of the cell, the third after stimulation/unloading. The unloading procedure was performed immediately following the acquisition of the second image. Unspecific destaining (ΔIᵤ) due to bleaching and passive diffusion of FM1-43 in the time interval between two stimuli was quantified by subtracting the second image from the first. The sum of specific and unspecific destaining (ΔIₜ) was determined by subtracting the third image (recorded after stimulation) from the second one (recorded before stimulation). The amount of specific destaining (ΔIₛ) was then calculated by subtracting ΔIᵤ from ΔIₜ. To identify all synapses independently of their exocytotic activity, the cultures from the FM1-43 experiments were fixed as described (Betz, 1998) and stained with a primary monoclonal antibody directed against Synaptophysin (Synaptic Systems) followed by an Alexa-488-labeled secondary antibody (Molecular Probes). Fluorescence images were taken from the same subcellular regions that were observed during FM1-43 recordings.

D) Image Analysis for Detection of Synapses

To quantify the number and size of fluorescent spots that represent synapses (FIG. 6), images of autaptic neurons stained for synaptic structures (Synaptophysin) and dendritic structures (MAP2) were analyzed as follows. Pixel noise in the image was first reduced by applying a ‘sharpening’ function, followed by low pass filtering. Then, synapses were distinguished from background fluorescence by setting a threshold that only shows objects above this threshold intensity. Objects smaller than 0.17 μm² were discarded, larger objects were detected by an object recognition algorithm and sorted by size. The average sized synapse was identified as the object that appeared at the highest frequency in the image. The proper threshold was calculated by a custom written algorithm that analyzed an image series with varying thresholds. Optimal threshold was determined when the total fluorescence intensity from the objects with highest occurrence (0.53±0.14 μm², 19 images, 20622 objects) was maximal. Objects were consitered to be synapses when their size was larger than 50% of the average synapse size. Spots whose summed fluorescence intensity was larger than twice the average were counted as two synapses.

To determine the ratio of active to inactive synapses in a given neuron (FIG. 6), superpositions of corresponding images from FM1-43 destaining experiments and immunostainings were analyzed manually. Synaptophysin containing boutons that stained with FM1-43 and destained upon stimulation were counted as active synapses while synaptophysin positive boutons that did not stain/destain in FM1-43 experiments were taken to be inactive.

E) Phosphorylation Experiments and Protein Analysis

Levels of PKCs were determined in brain homogenates of wild type and Munc13-1^{H567K}/Munc13-1^{H567K} mice by SDS-PAGE (Laemmli, 1970), followed by transfer of separated proteins to nitrocellulose (Towbin, 1979), and immunoblotting using commercially available antibodies (Transduction Laboratories). Immunolabeled bands were visualized by enhanced chemoluminescence (ECL; Amersham Pharmacia Biotech) and quantified densitometrically. For the analysis of β-PE dependent phosphorylation of proteins in cultured neurons, cortical/hippocampal neurons from wild type and Munc13-1^{H567K}/Munc13-1^{H567K} brains were plated at high density on polylysine/collagene. For radioactive labeling, cells were washed twice with phosphate free MEM-Eagle medium and incubated in this medium for 2 h in the presence of 0.2 mCi/ml ³²P-orthophosphate (Amersham Pharmacia Biotech). Cells were then stimulated with 1 μM PDBU for 1 h, washed in phosphate free MEM-Eagle medium and harvested in extraction buffer (1% sodium cholate, 100 mM NaCl, 2 mM EGTA, 25 mM HEPES-KOH, pH 7.4, 5 μM microcystin LR, 20 mM NaF, 20 mM Na-pyrophosphate, 1 mM p-nitrophenyl phosphate, 1 μg/ml aprotinin, 0.5 μg/ml leupeptin, 0.2 mM phenylmethylsulfonyl fluoride) for 2D-electrophoresis or immunoprecipitation experiments. 2D-Electrophoresis (isoelectric focusing/SDS-PAGE) was performed using IPG-strips (pH 3-10; Amersham Pharmacia Biotech; Görg, 1988) in an IPGphor Isoelectric Focusing System (Amersham Pharmacia Biotech) for the first dimension and the Mini Protean II System (Bio-Rad) for the second dimension. Gels were dried and analyzed by autoradiography. Immunoprecipitation of Munc13-1 was done as described with a specific polyclonal antibody (N395; Betz, 1997). Separated proteins were blotted to nitrocellulose and analyzed by immunoblotting with a monoclonal antibody to Munc13-1 (Betz, 1998) and ECL (Amersham Pharmacia Biotech). Immunoprecipitated Munc13-1 was quantified densitometrically. Phosphorylation of SNAP-25 and GAP43 was analyzed by stimulating cortical/hippocmpal neurons with 1 μM PDBU for 30 min. Cells were then washed with PBS and harvested for direct analysis by SDS-PAGE and immunoblotting using phosphopeptide specific antibodies (Iwasaki, 2000; Kawakami, 2000).

### EXAMPLE 2

### Generation and Basic Characteristics of Munc13-2 Deletion Mutant Mice

Munc13-2 deletion mutant mice were generated as described in Example 1. Southern blot analysis of offspring obtained by interbreeding heterozygous Munc13-2 deletion mutants showed that the respective genotypes (+/+, +/−, −/−) were present at the expected Mendelian frequency (FIG. 1B). Western blot analyses of brain homogenates from these mice demonstrated that Munc13-2 protein expression is completely abolished in homozygous mutants and reduced by about 50% in heterozygous littermates (FIG. 1C). Apart from sporadic seizures in older animals (>1 yr) the overall phenotype of Munc13-2 deletion mutants was very similar to that of wild type littermates. Homozygous mutants were viable and fertile and had no morphological abnormalities. In particular, the brains of Munc13-2 deficient mice showed a normal cell density, cytoarchitecture, connectivity, distribution/density of synapses, and ultrastructural morphology (not shown). In addition, the levels of all tested synaptic proteins (Munc13-1, Munc13-2, α-SNAP, Complexin I, Complexin II, Munc18-1, NSF, Rab3A, SNAP-25, Synapsin I/IIa, Synapsin IIb, Synaptobrevin 2, Synaptophysin, Synaptotagmin I, Syntaxin 1) were unaffected by the lack of Munc13-2 (not shown). Thus, like Munc13-3 (Augustin, 2001) but in contrast to Munc13-1 (Augustin, 1999b), Munc13-2 is not essential for normal physiological function.

### EXAMPLE 3

### Basic Synaptic Properties of Munc13-2 Deficient and Munc13-1/2 Double Deficient Neurons

Previously, it was demonstrated that Munc13-1 is an essential priming factor for most excitatory synapses in hippocampal cultures, while inhibitory synapses are apparently independent of Munc13-1 function (Augustin, 1999b). To study the role of Munc13-2, the only other Munc13 isoform expressed in hippocampus (Augustin, 1999a), in excitatory and inhibitory synaptic transmission, and to examine the degree of redundancy between Munc13 isoforms, excitatory and inhibitory synaptic transmission in Munc13-2 deficient and Munc13-1/2 double deficient hippocampal neurons in comparison to wild type and Munc13-1 deficient cells were analyzed. Cultured single autaptic neurons (9-16 days in culture) from embryonic (E18) or newborn (P0) mice were employed. Synaptic currents from voltage clamped neurons held at −70 mV were evoked by 2 ms depolarization to 0 mV.

Excitatory postsynaptic currents (EPSCs) from wild type and Munc13-2 deficient neurons were not significantly different. EPSCs were 5.0±0.8 nA, n=10, for wild type cells and 5.2±0.8 nA, n=19, for Munc13-2 deficient cells (FIGS. 3A and 3B). Likewise, elimination of Munc13-2 had no apparent effect on inhibitory synaptic transmission. IPSCs were 3.5±0.9 nA, n=13, for wild type cells and 3.5±0.8 nA, n=5, for Munc13-2 deficient cells (FIGS. 4A and 4B). mEPSC and mIPSC amplitudes and frequencies were also not affected by the Munc13-2 deletion (see examples in FIGS. 3D and 4C). In addition, the readily releasable vesicle pool of Munc13-2 deficient neurons was unchanged as determined by application of hypertonic sucrose solution (0.94±0.21 nC, n=10, for wild type excitatory neurons vs. 0.93±0.14 nC, n=10, for Munc13-2 deficient excitatory cells; FIG. 3C; data for inhibitory neurons not shown).

In striking contrast to the elimination of Munc13-2, deletion of Munc13-1 leads to a complete shut down of the majority of synapses formed by a single excitatory neuron, resulting in 90% reductions of readily releasable vesicle pools and evoked transmitter release (FIGS. 3A-C; see also Augustin, 1999b). Unless neurons can utilize alternative, Munc13 independent pathways for vesicle priming, the differences between the consequences of the Munc13-1 and Munc13-2 deletions suggest a partial redundancy of Munc13 isoforms in hippocampal neurons. In excitatory cells, Munc13-1 can compensate for loss of Munc13-2 but not vice versa. In contrast, Munc13-1 and Munc13-2 are entirely redundant in inhibitory neurons.

In order to overcome the problem of redundancy between Munc13 isoforms in cultured hippocampal neurons, Munc13-1/2 double deficient mice were generated by interbreeding animals homozygous for the Munc13-2 mutation and heterozygous for the Munc13-1 mutation (Augustin, 1999b). In offspring from such interbreedings, homozygous Munc13-2 single mutants, which are indistinguishable from wild type (see above) and served as control in all subsequent analyses, and Munc13-1/2 double deletion mutants, which expressed neither Munc13-1 nor Munc13-2 (FIG. 1D), were present at similar frequencies. Munc13-1/2 double mutants were very fragile and often born dead. All double mutants that were born alive died within 1 h after birth. However, hippocampi from double mutant embryos and newborn pups showed normal cell density and cytoarchitecture (not shown). For these reasons, we used embryonic (E18) double mutant mice and Munc13-2 deficient litter mates for culturing hippocampal neurons. It was found that Munc13-1/2 double deficient neurons developed a normal cell morphology in culture (see example in FIG. 6A). Whole cell recordings from these neurons showed normal voltage dependent Na⁺, K⁺ and Ca²⁺ currents (not shown). However, synaptic events were completely absent. From a total of 45 recorded neurons, none showed any sign of evoked or spontaneous release (FIGS. 3 and 4). Moreover, all recorded neurons were insensitive to application of hypertonic sucrose solution, indicating a complete absence of release competent vesicles. These data allow several important conclusions: 1. Since release by hypertonic shock does not require Ca²⁺ channel activity (Rosenmund, 1996), the inability of Munc13-1/2 double deficient neurons to release transmitter is not due to effects of the double mutation on the Ca²⁺ triggered release step (see Augustin, 1999b for corresponding data on Munc13-1 single mutants). Rather, the complete absence of spontaneous, hypertonically induced, or evoked release in Munc13-1/2 double deficient cells suggest a complete loss of fusion competent vesicles (see Augustin, 1999b for corresponding data on Munc13-1 single mutants). 2. As all modes of vesicular release (spontaneous, action potential evoked, hypertonically evoked) are equally abolished in Munc13-1/2 double mutant cells, they depend on the same vesicular priming process and draw from the same pool of release competent, primed vesicles, suggesting that hypertonically induced responses are a faithful measure for the pool of fusion competent vesicles. 3. Assuming that no developmental problems in synaptogenesis contribute to the Munc13-1/2 double mutant phenotype (see Examples below), no priming factors other than Munc13s exist in hippocampal neurons.

Although the usual discrimination of inhibitory and excitatory neurons by response shape and pharmacological characteristics was impossible in Munc13-1/2 double mutant neurons, it is presumed that inhibitory cells were equally affected by deletion of Munc13-1 and -2 because staining of Munc13-1/2 double deficient cultures with antibodies to glutamic acid decarboxylase (GAD) revealed normal frequency of GAD positive cells (approx. 20%; not shown). Thus, the likelihood that none of the 45 recorded cells was inhibitory is extremely low. More importantly, strong stimulation of release by α-latrotoxin at the end of each experiment allowed the successful induction of very rare mPSC events (see below) and thus identification of transmitter phenotypes on the basis of mPSC shape, amplitude and pharmacology. Using this approach, 5 of a total of 45 recorded cells were shown to be GABAergic.

Munc13-1 deletion mutant neurons are characterized by a dramatic reduction in readily releasable vesicle pools and evoked transmitter secretion. Interestingly, this phenotype is specific for glutamatergic neurons while GABAergic cells remain completely unaffected by the lack of Munc13-1. Within a given Munc13-1 deficient glutamatergic neuron, a subpopulation of glutamatergic synapses appears to be completely shut down whereas the remaining synapses release with normal release probability (Augustin, 1999b). In contrast to Munc13-1, deletion of Munc13-3, the only Munc13 isoform that is coexpressed with Munc13-1 in cerebellum, has only very mild phenotypic consequences that are not compatible with an essential role of Munc13-3 in GABAergic synaptic vesicle priming (Augustin, 2001). Experiments shown herein demonstrate that the same is true for Munc13-2 (FIGS. 3 and 4). Thus, neither Munc13-2 nor Munc13-3 are as essential for GABAergic transmission as Munc13-1 is for glutamatergic cells. In the past, the surprising lack of phenotypic alterations in Munc13-2 and Munc13-3 deletion mutants has led us to speculate that the specificity of Munc13-1 mediated vesicle priming for glutamatergic synapses is unlikely to be caused by simple compensatory mechanisms involving Munc13-2 or Munc13-3, but rather due to the fact that GABAergic neurons utilize a different, Munc13 independent priming mechanism (Augustin, 1999b). The current study demonstrates clearly that this is not the case in hippocampal neurons where the only two Munc13 isoforms present, Munc13-1 and Munc13-2, exhibit a complex pattern of redundancy. In GABAergic hippocampal neurons, the loss of Munc13-1 can be compensated by the presence of Munc13-2 and vice versa (FIG. 4), indicating a high degree of redundancy. In glutamatergic hippocampal neurons, on the other hand, Munc13-1 can functionally compensate for Munc13-2 loss but not vice versa (FIG. 3). In both cell types, the complete absence of Munc13 priming factors leads to a total arrest of synaptic vesicle priming and elimination of readily releasable vesicles (FIGS. 3 and 4), demonstrating that Munc13 mediated vesicle priming is a common and essential feature of two different fast neurotransmitter systems. Interestingly, recent data on the role of Munc13 proteins in catecholamine secretion from adrenal chromaffin cells suggest that Munc13s may also be involved in the priming of chromaffin granules (Ashery, 2000). It is, therefore, proposed that Munc13 mediated vesicle priming is an essential part of all regulated secretory processes.

The present study (FIG. 4) demonstrate that Munc13-1 and Munc13-2 are completely redundant in GABAergic hippocampal neurons. This suggests that in wild type GABAergic neurons, all synapses are equipped with both, Munc13-1 and Munc13-2. However, this is not the case for glutamatergic cells which apparently form two types of synapses that are differentially equipped with Munc13-1 and Munc13-2. Currently available antibody tools for the detection of Munc13-1 and Munc13-2 are insufficient such that a direct comparison of the synaptic distribution of these two proteins in our cultures has not been possible. Nevertheless, the FM1-43 imaging experiments shown herein above showed that the distribution of Munc13-1 independent synapses did not follow any obvious pattern such as preferential somatic or dendritic localization. It is suggested that the heterogeneity of synapses is intrinsically present. This view is supported by data from semiquantitative immunoelectron microscopy studies showing that Munc13-1 is only present in 45% and 56% of type I (excitatory) presynaptic compartments in the parietal cortex and stratum radiatum of hippocampus, respectively (Betz, 1998). This differential synapse specific localization of Munc13-1 is striking because in situ hybridization demonstrated that almost all neurons in these regions, and in the brain in general, express Munc13-1 mRNA (Augustin, 1999a). Thus, the morphological data on the ultrastructural distribution of Munc13-1 suggest that the heterogeneity of synapses with respect to Munc13-1 dependence in cultured neurons is not artifactual. Rather, glutamatergic neurons in situ likely express a similarly heterogeneous synapse population with respect to Munc13 isoform content and dependence. That individual axons in situ can indeed form functionally different types of synapses is also evident from experiments in cortical (Reyes, 1998; Thomson, 1997) and hippocampal (Scanziani, 1998) slices.

### EXAMPLE 4

### Normal Postsynaptic Responsiveness Despite Complete Block of Release in Munc13-1/2 Double Deficient Neurons

Given that neither action potentials nor hypertonic stimuli successfully evoked synaptic transmission and no mPSCs were detectable in Munc13-1/2 double deficient cells, it is possible that postsynaptic development is impaired in mutant cells. To test this directly, the spider toxin α-latrotoxin was used which allows to partially circumvent the need for priming factors in vesicular release (Augustin, 1999b). It was found that application of 1 nM α-latrotoxin induced mEPSC and mIPSC activity in Munc13-1/2 double mutant neurons, but the release rate was much lower than that of wild type and Munc13-2 single mutant control cells (FIGS. 5A-D). Five minutes following a 60 s application of 1 nM α-latrotoxin, Munc13-2 single mutant control neurons exhibited mEPSCs at a frequency of 23.4±3.9 Hz (n=17) (FIGS. 5A and 5B). The average amplitude of these events was 22.7±2.4 pA (FIG. 5B). Both, mEPSC frequency and amplitude values are similar to wild type values (not shown). In contrast, Munc13-1/2 double deficient neurons showed mEPSC-like events after α-latrotoxin treatment at a frequency of only 0.074±0.024 Hz (n=17) (FIGS. 5A and 5B). However, the amplitude of these rare mEPSC events was comparable to that observed in wild type and Munc13-2 single mutant neurons (25.6±2.6 pA, n=17; FIG. 5B). Similarly, mIPSC-like events in the presence of α-latrotoxin were observed in five Munc13-1/2 double deficient cells at low frequency but with amplitudes comparable to wild type values (FIGS. 5C and 5D).

The facts that mEPSC and mIPSC amplitudes were not affected in the Munc13-1/2 double mutant background (FIGS. 5B and 5D) and that the detected events were blocked by the appropriate postsynaptic antagonists (not shown) suggest that postsynaptic equipment with and accumulation of functional AMPA or GABA receptors is not impaired. This conclusion was supported by the observation that the localization of the glutamate receptor subunit GluR2 in Munc13-1/2 double mutant neurons was indistinguishable from control data and largely overlapped with that of the presynaptic marker Synaptophysin (not shown). In addition to the apparently normal incorporation of postsynaptic receptors, the overall density of receptors was normal in Munc13-1/2 double mutant neurons as determined by the responses of double mutant and control cells to applications of 10 μM kainate and 1 μM GABA (FIG. 5E).

### EXAMPLE 5

### Normal Density and Ultrastructure of Synapses in Munc13-1/2 Double Deficient Neurons

To exclude the possibility that the dramatic reduction in all modes of transmitter release from cultured Munc13-1/2 double deficient neurons is caused by an inability of mutant cells to form sufficient numbers of structurally intact synapses, synapse density and ultrastructure in cultures from double mutant and control hippocampi were examined. Autaptic cultures were maintained in vitro for 10-12 days and then stained for the dendritic marker MAP2 and the presynaptic marker Synaptophysin using specific primary and fluorescent secondary antibodies. The total number of Synaptophysin positive synapses as well as the density of synapses per μm² MAP-2 positive dendrite were then determined with automatic synapse detection algorithms. Both, the total number of synapses per cell (not shown) and the synapse density were very similar in Munc13-2 control and Munc13-1/2 double mutant neurons (FIGS. 6A-C). Thus, synapse formation in culture as determined by these light microscopic criteria is not altered upon complete shut down of transmitter release in Munc13-1/2 double deficient neurons.

Next, the ultrastructure of Munc13-1/2 double deficient synapses was examined in order to detect any effects of the loss of Munc13 priming factors or lack of synaptic activity on synapse assembly. For this purpose hippocampal neurons from control Munc13-2 deficient and Munc13-1/2 double deficient mice were cultured at high density. After 12 days in vitro, cells were processed for standard electron microscopic analysis (Augustin, 1999b). No change of synapse structure in Munc13-1/2 double mutant neurons were observed (FIGS. 6D and 6E), supporting previous observations made in Munc13-1 deletion mutant neurons where loss of the essential priming factor Munc13-1 had no effect on synapse structure (Augustin, 1999b). Taken together, these data demonstrate that the striking phenotype of Munc13-1/2 double mutant neurons is not caused by a more general dysfunction in synapse assembly. In addition, the present data set shows that synapses assemble normally even in the complete absence of spontaneous and evoked transmitter release activity.

This study extends published data on Munc18-1 deficient mice in which transmitter release is also completely shut down (Verhage, 2000). Synapses in Munc18-1 deficient brains are initially formed normally during brain development but degenerate as the embryo matures. At birth, higher brain structures such as the hippocampus and cerebral cortex appear morphologically intact while lower structures such as the spinal cord and most parts of the brain stem are completely degenerated. On the basis of these observations, it was concluded that synaptic transmitter release is not necessary for the initial formation of synapses but essential for their maintenance (Verhage, 2000). In contrast to the Munc18-1 deletion mutants, here no evidence was found for degenerative processes in the completely release incompetent Munc13-1/2 double deficient brains. In particular, spinal cord and brainstem, which more or less exclusively express Munc13-1 and Munc13-2 and not Munc13-3 (Augustin, 1999a and 2001), were intact (not shown). These observations, together with data on synaptogenesis and synaptic characteristics of Munc13-1/2 double deficient neurons in culture (FIGS. 5 and 6) demonstrate that synaptic activity is not only dispensable for initial synaptogenesis but also not necessary for the maintenance of synapses and many of their basic characteristics. The degeneration observed in Munc18-1 deficient brains may therefore be due to the perturbation of a Munc18-1 dependent process that is not directly related to transmitter secretion.

### EXAMPLE 6

### No Endocytotic Vesicle Recycling in Munc13-1/2 Double Deficient Neurons

Since no signs of release in Munc13-1/2 double deficient synapses was detected, it was next tested whether this lack of release capacity affects the synapses' ability to endocytose plasma membrane components. Stimulation induced uptake and release of the styryl dye FM1-43 was assayed with quantitative fluorescence microscopy to follow synaptic exo- and endocytosis in Munc13-2 single mutant control and Munc13-1/2 double deficient neurons (Ryan, 1996; Murthy, 1997; Cochilla, 1999). Exocytosis was stimulated by high K⁺ mediated depolarization, and FM1-43 was added to trace the subsequent endocytotic uptake of membrane. Cells were then washed and the remaining membrane staining was used as a measure of endocytosis. The specific loss of FM1-43 staining during a second high K⁺ mediated depolarization was then used to determine the degree of release of previously endocytosed dye (FIG. 7; see Example 1 for details of the procedure). It was found that Munc13-2 deficient neurons showed fluorescence increases (staining/endocytosis) and losses (destaining/exocytosis) that are typical for our culture system (FIG. 7, left). In contrast, Munc13-1/2 double deficient synapses neither stained nor destained specifically in the presence of FM1-43 (FIG. 7, right), indicating that these double mutant cells are not only unable to exocytose but also lack Ca²⁺ triggered endocytotic activity. Assuming that Munc13s are not directly involved in endocytosis, these data show directly that synaptic endocytosis is coupled to exocytosis and does not take place when exocytosis is arrested.

### EXAMPLE 7

### Distinct Populations of Munc13-1 Dependent and Munc13-1 Independent Synapses in Individual Excitatory Neurons

Previous, it was shown that glutamatergic Munc13-1 deficient neurons, like the Munc13-1/2 double mutant neurons studied here, form normal numbers of synapses (Augustin, 1999b). These Munc13-1 deficient neurons were characterized by a 90% reduction in readily releasable vesicle pools and evoked transmitter release. Interestingly, indirect functional and pharmacological assays to determine the fraction of active synapses in Munc13-1 mutants showed that the dramatic 90% deficit in transmitter release competence in Munc13-1 mutant cells was not due to an equal reduction in all synapses. Rather, 10% of glutamatergic Munc13-1 deficient synapses transmitted with normal release characteristics while the majority of synapses appeared to be completely shut down (Augustin, 1999b). To show this directly, the fraction of active synapses in Munc13-1 deficient neurons was measured by assaying the uptake and release of the fluorescent membrane dye FM1-43 with quantitative fluorescence microscopy (Cochilla, 1999).

Exocytotically active synapses were identified on fluorescence images from selected subregions of autaptic neurons by measuring the stimulation induced decrease of FM1-43 fluorescence intensity (see Example 1). In each experiment, loading and unloading of FM1-43 were performed twice, first with action potential trains and second with K⁺ induced depolarization. Both data sets were analyzed independently and yielded similar activity patterns. The total synapse population was determined retrospectively by immunocytochemical staining for Synaptophysin and counting of synapses in the same regions that were examined in the FM1-43 experiments. Verifying previous results (Augustin, 1999b), it was found that the density of immunocytochemically defined Synaptophysin positive synapses did not differ significantly between Munc13-1 deficient and wild type cells (not shown). However, the fraction of active synapses, which is the ratio between the number of active synapses and the total Synaptophysin positive synapse count, was 66.3±18% for wild type cells but only 9.4±3.6% in Munc13-1 deficient neurons (FIGS. 8A-8E). This indicates that in excitatory/glutamatergic Munc13-1 deficient neurons, only a small subpopulation of synapses is active while the majority is silent. The present data indicate that this pool of active synapses in Munc13-1 deficient neurons is primed by Munc13-2 because it is not detectable in Munc13-1/2 double deficient cells.

In earlier experiments on Munc13-1 deficient hippocampal neurons, it was demonstrated that the overall readily releasable vesicle pools were dramatically reduced which, in turn, caused a strong reduction in evoked transmitter release. Interestingly, the remaining release activity in Munc13-1 deficient glutamatergic neurons exhibited apparently normal synaptic release probability (Augustin, 1999b). The observation of normal synaptic release probabilities despite dramatically reduced readily releasable vesicle pools suggested that in Munc13-1 deficient excitatory neurons the majority of synapses is completely shut down while a small subpopulation functions normally. Indirect support for this view was obtained in pharmacological experiments involving use dependent blockade of synaptic NMDA receptors by MK-801. In these experiments, it was found that in Munc13-1 deficient cells only a small subset of synaptic NMDA receptors (20%) is activated and subsequently blocked by MK-801 during synaptic activity. In contrast, the same treatment in wild type cells led to the activation and subsequent MK-801 mediated block of most cellular NMDA receptors (75%) (Augustin, 1999b). In the present study, direct evidence is provided for the view that glutamatergic hippocampal neurons form two types of synapses. Using FM1-43 to detect active synapses and retrospective immunostaining for Synaptohysin to detect all synapses it was found that the fraction of active synapses (the ratio between the number of active synapses and the total Synaptophysin positive synapse count in the same field) in wild type cells was seven times higher than that observed in Munc13-1 deficient neurons (FIGS. 8A-8E).

Interestingly, the synaptic release activity that is detectable in Munc13-1 deficient neurons is completely abolished when Munc13-2, the only other Munc13 isoform expressed in hippocampus, is also deleted (FIGS. 3 and 4). Thus, active synapses in Munc13-1 deficient neurons are exclusively driven by Munc13-2 and should represent an ideal tool to study the characteristics of Munc13-2 mediated vesicle priming in isolation. Indeed, a detailed analysis of such Munc13-2 driven synapses in glutamatergic neurons revealed striking differences to Munc13-1 driven synapses with respect to short term plasticity, suggesting that the differential dependence of synapses on Munc13 priming factors may have important consequences for complex neural mechanisms in the central nervous system, as documented herein below.

### EXAMPLE 8

### Reawakening of Silent Munc13-1/2 Double Deficient Synapses by Viral Overexpression of Munc13-1 or Munc13-2/Munc13-1 Independent Synapses Empoly Munc13-2 as a Priming Factor

Although Munc13-1 is the dominant isoform in hippocampus, western blot analysis revealed an additional Munc13 isoform, Munc13-2 (Augustin, 1999a; 2001). In order to examine whether Munc13-1 independent synapses employ Munc13-2 as an alternative priming factor, Munc13-2 deficient mice were crossed with Munc13-1 mice to produce Munc13-1/2 double deficient synapses. Cultured neurons from these double deficient mice developed normal as judged from gross morphological inspections. These neurons, however, displayed no evoked synaptic responses, when stimulated with action potentials (n=45), indicating that the remaining isoform underlying the response from Munc13-1 deficient neurons relies on the priming function of Munc13-2 (FIG. 9A). To test whether the loss of synaptic transmission is not due to an additional defect besite the lack of a Munc13 priming factor, both hippocampal Munc13 were overexpressed isoforms in the Munc13-1/2 double deficient neurons. Munc13-1 or Munc13-2 were reintroduced using Semliki forest virus expression vectors (REF). Treatment with both Munc13 isoforms rescued synaptic transmission in the release incompetent neurons with high efficiency (FIG. 9). EPSC amplitudes of the wild type like Munc13-2 deficient neurons were 1.3±0.3 nA (n=17), while Munc13-1/2 double deficient neurons overexpressing Munc13-1 and Munc13-2 showed EPSC amplitudes of 0.84±0.14 nA (n=25) and 0.78±0.11 nA (n=32), respectively (FIGS. 9A and 9B). Similar rescue efficiencies were observed in inhibitory neurons although a smaller number of cells were examined (FIGS. 9C-9D). These data show that Munc13 deficient, release incompetent neurons can be rescued to a wild type like phenoptype by virus mediated reintroduction of Munc13 proteins. The fact that rescued EPSC amplitudes approach wild type levels irrespective of the Munc13 isoform suggests that all preformed synapses accept both Munc13 isoforms as a priming factor. This is remarkable because in wild type neurons, 90% of all synapses are exclusively dependent on Munc13-1 and therefore do not express Munc13-2 ((Augustin, 1999b) and FIGS. 8A-8E). In summary, these data show that both Munc13 isoforms functions in wildtype hippocampus in different synapse population.

### EXAMPLE 9

### Munc13-1 Dependent and Munc13-2 Dependent Synapses Exhibit Different Types of Short Term Synaptic Plasticity During High Frequency Stimulation

Reproducing our previous observations (Augustin, 1999b), it was found found that at low stimulation frequencies (0.2 Hz), EPSCs measured in Munc13-1 deficient neurons are dramatically reduced (107±17 pA, n=56, versus 1.28±0.25 nA, n=45, in wild type or heterozygous mutant neurons; see also (Augustin, 1999b)). This reduction is due to a selective priming deficit in the majority (90%) of synapses formed by a single axon of Munc13-1 deficient cells (see above). The remaining 10% of synapses in Munc13-1 deficient neurons employ Munc13-2 as a priming factor, and are therefore a good system to study the function of Munc13-2. Munc13-2 dependent synapses release transmitter with an apparently wild type like release probability when stimulated at low frequency (Augustin, 1999b). However, Munc13-2 driven synapses were found to differ profoundly from Munc13-1 dependent synapses when analyses were performed at higher stimulation frequencies. Typically, EPSCs in wild type, Munc13-1 dependent synapses depress with time when stimulated at frequencies between 1-20 Hz. Indeed, at action potential frequencies of 10 Hz, EPSC amplitudes of wild type neurons decreased steadily over 100 stimuli to 32% of the initial amplitude (1170±250 pA to 373±121 pA, n=24; FIG. 8A). EPSCs of Munc13-1 deficient, decreased wild type like for the first two consecutive responses (FIG. 10D). However, the EPSC amplitude then increased (augmented) to about 200% of the baseline value during 10 stimuli (from 78±16 pA to 147±15, n=47). After 100 stimuli, the EPSC amplitude in Munc13-1 deficient cells was still 70% larger than the initial amplitude (121±15 pA, n=47; FIG. 8B). Despite this strongly augmenting behavior of Munc13-1 deficient neurons, the steady state amplitude of EPSCs during high frequency stimulation remained below 30% of the wild type value (FIG. 10A). Because this remaining release component in Munc13-1 deficient neurons is not detectable in the completely release incompetent Munc13-1/Munc13-2 double deficient neurons, it can be conclude that it is maintained by Munc13-2 mediated vesicle priming.

In contrast to excitatory/glutamatergic synapses, Munc13-1 deficient inhibitory/GABAergic cells showed the typical wild type like depression of IPSCs during 10 Hz stimulation (2.0±0.5 nA to 0.36±0.092 nA, n=10, for wild type cells and 2.12±0.34 nA to 0.64±0.11 nA, n=27, for Munc13-1 deficient cells; FIG. 3C). This finding supports our previous observation (Augustin, 1999b) that loss of Munc13-1 has little effect on inhibitory neurotransmission, most likely because Munc13-2 is coexpressed with Munc13-1 in all GABAergic synapses.

To verify that augmenting phenotype in Munc13-1 deficient neurons is strictly coupled to the presence of Munc13-2 and not due to culture artifacts (the neurons have 90% less active synapses than wildtype neurons), Munc13-1/Munc13-2 double deficient neurons were examined after Semliki Forest Virus mediated reintroduction of Munc13-1 or ubMunc13-2.). Cells rescued with the ubMunc13-2 isoform showed the EPSC augmentation during 10 Hz stimulation that is characteristic of Munc13-1 deficient neurons in which Munc13-2 is assumed to drive vesicle priming (compare FIGS. 10B and 10E). Conversely, cells rescued with the Munc13-1 isoform showed a depression of EPSC amplitudes during 10 Hz stimulation that is also typically seen in wild type and Munc13-2 deficient cells (compare FIGS. 10B and 10E). These data demonstrate that overexpression of Munc13-1 or ubMunc13-2 in Munc13-1/Munc13-2 double deficient, priming incompetent hippocampal neurons creates phenocopies of wild type/Munc13-2 deficient and Munc13-1 deficient neurons, respectively. This observation lends additional support to the view that synaptic release activity in wild type or Munc13-2 deficient cells is driven by Munc13-1 while synaptic transmitter release in Munc13-1 deficient cells is due to the priming action of Munc13-2 only. Thus, the striking differences in short term plasticity between wild type and Munc13-1 deficient neurons (FIG. 10 and additional data below) is due to functional differences between Munc13-1 and Munc13-2, which differentially control changes in release dynamics during high frequency action potential trains. Closer examination of the degree of depression during 10 Hz trains in wild type neurons and Munc13-1/Munc13-2 double deficient cells overexpressing Munc13-1 indicated that double mutant neurons rescued with Munc13-1 depressed more strongly than wild type cells (FIG. 10F). The time course of the computed difference amplitude between wild type and Munc13-1 rescue after normalization had a shape that was similar to the one obtained in Munc13-2 dependent synapses, suggesting that Munc13-2 dependent synapses contribute as expected from the Munc13-1 deficient mice to total steady state responses in wild type neurons.

The differential expression of Munc13 isoforms in these two synapse populations has striking functional consequences. Munc13-2 dependent synapses facilitate during trains of action potentials and show augmentation following high frequency stimulation while Munc13-1 dependent synapses exhibit depression and no augmentation under the same stimulation conditions (FIG. 10). Thus, beyond simply acting as essential vesicle priming factors (Aravamudan, 1999; Augustin, 1999b; Richmond, 1999), the characteristics and distribution of Munc13 isoforms determine short term plasticity properties of individual synapses.

The differential equipment of synapses with Munc13 isoforms is unlikely to be an artifact of the culture system used in the present and previous studies (Augustin, 1999b). Semiquantitative immunoelectron microscopy studies showed that Munc13-1 is only present in 45% and 56% of type I (excitatory) presynaptic compartments in the parietal cortex and stratum radiatum of hippocampus, respectively (Betz, 1998). This differential synapse specific localization of Munc13-1 is striking because in situ hybridization demonstrated that almost all neurons in these regions, and in the brain in general, express Munc13-1 mRNA (Augustin, 1999a). Thus, the morphological data on the ultrastructural distribution of Munc13-1 suggest that the heterogeneity of synapses with respect to Munc13-1 dependence in cultured neurons is not artifactual. Rather, glutamatergic neurons in situ likely express a similarly heterogeneous synapse population with respect to Munc13 isoform content and dependence.

That individual axons in situ can indeed form functionally different types of synapses is evident from experiments in cortical (Reyes, 1998; Thomson, 1997) and hippocampal (Scanziani, 1998) slices. These studies showed that a single axon can form both, facilitating and depressing synapses, features mirroring the functional differences between Munc13-1 and Munc13-2 dependent synapses observed in this study. In some cases this heterogeneity is dependent on the target cell type (Reyes, 1998). In addition, neurotrophic factors (Asztely, 2000; Davis, 1994) and developmental changes control the differential formation of facilitating and depressing synapses (Pouzat, 1997; Reyes, 1999). In view of the present data and the essential role of Munc13 mediated vesicle priming for synaptic transmission (Augustin, 1999b), it is proposed that at least in some cases where functionally distinct facilitating and depressing synapses are observed in vivo, their differential equipment with Munc13 priming factors is causing the functional differences. These data suggest a simple mechanism, i.e. switch from Munc13-1 to Munc13-2 in the presynaptic terminal or vive versa, by which a depressing or facilitating synaptic phenotype could be transformed into the respective other.

### EXAMPLE 10

### Munc13-1 and Munc13-2 Dependent Synapses Exhibit Different Types of Short Term Synaptic Plasticity Following High Frequency Stimulation

In order to determine which release parameters are differentially affected by Munc13-1 and Munc13-2 during high frequency action potential trains, synaptic amplitudes (stimulated at 0.2 Hz) immediately after a 10 Hz action potential train were examined. It was found that in wild type neurons, EPSCs (n=27) and IPSCs (n=18) recovered from depression to baseline values within one or two post train stimuli (FIG. 11A). In striking contrast, glutamatergic Munc13-1 deficient neurons showed a dramatic increase of EPSC amplitudes 2 s following the 10 Hz train, reaching 578±108 pA (n=45), representing a more than fivefold augmentation over basal EPSC amplitudes (FIG. 11B). Munc13-1 deficient inhibitory/GABAergic cells also showed a significant, albeit much more subtle degree of IPSC augmentation (FIG. 4B, bottom), providing the first evidence for an effect of the Munc13-1 deletion on GABAergic neurons (see Augustin, 1999b). Here, the baseline amplitude was 1.45±0.43 nA, and following the 10 Hz action potential train, amplitudes were augmented to 2.09±0.49 nA (n=12).

In order to verify that the differences in post train EPSC augmentation between wild type and Munc13-1 deficient neurons are really due to functional differences between Munc13-1 and Munc13-2, which drive vesicle priming in the respective neurons, rescue experiments with Munc13-1/Munc13-2 double deficient neurons. Were employed. As with short term plasticity characteristics during high frequency action potential trains, it was found that Semliki Forest Virus mediated overexpression of Munc13-1 generated a phenocopy of non augmenting wild type/Munc13-2 deficient cells while overexpression of ubMunc13-2 resulted in an EPSC augmenting phenotype that closely resembled that of Munc13-2 driven Munc13-1 deficient neurons (FIGS. 11 C and D). Taken together, these data demonstrate that wild type or Munc13-2 deficient neurons serve as ideal models to study Munc13-1 mediated vesicle priming, while Munc13-1 deficient neurons allow the analysis of Munc13-2 function in isolation. It was concluded that the differences in short term plasticity of synaptic transmission between wild type and Munc13-1 deficient neurons are mainly, if not exclusively, due to functional differences between Munc13-1 (depressing) and Munc13-2 (augmenting). The post train EPSC augmentation in Munc13-2 synapses is transient (FIGS. 11B top and 11C), and augmented EPSCs returned to baseline with a time constant of 5-18 s (8.3±2.1 s; n=45). These characteristics (identical basal release probability, delayed onset of amplitude increase, time course of decay to baseline) are indeed consistent with forms of short-term plasticity that are classically defined as augmentation (Delaney and Tank, 1994; Magleby and Zengel, 1976; Nussinovitch and Rahamimoff, 1988; Zucker, 1973). Therefore, Munc13-2 dependent synaptic transmission in Munc13-1 deficient neurons was used as a model for the analysis of augmentation mechanisms. To detect a possible postsynaptic contribution to the augmentation observed in Munc13-1 deficient, Munc13-2 dependent cells, augmentation of the slow NMDA component of the EPSC was first compared with the fast AMPA component. To measure both components simultaneously, EPSCs were stimulated in the presence of 10 μM glycine and 2.7 mM Ca²⁺ but in the absence of Mg²⁺. The transmitter release probability under these conditions was slightly higher than under our standard conditions (4 mM Ca²⁺/4 mM Mg²⁺). Augmentation was induced with a 10 Hz action potential train, and the dual component EPSC was examined before and 2 s after augmentation. Presumably due to the elevated release probability, augmentation was generally less pronounced than under standard ionic conditions, but the degree of augmentation was indistinguishable between the AMPA (2.4±0.2 fold, n=14) and NMDA components (2.57±0.13 fold, n=9) (not shown). In additional experiments, potential changes in postsynaptic sensitivity during augmentation (e.g. due to postsynaptic insertion of new receptors) were tested by probing the total responsiveness of postsynaptic AMPA receptors in Munc13-2 dependent cells. It was expected that this approach would allow us to reliably detect increases in postsynaptic receptor density or sensitivity due to augmentation, because a large fraction of cellular AMPA receptors is synaptically localized (Craig and Boudin, 2001) and an average EPSC augmentation of some 500% would therefore be paralleled by a readily detectable increase in cellular responses to exogenously added AMPA receptor agonists. However, when responses of Munc13-1 deficient, Munc13-2 dependent cells were compared to exogenously applied kainate (10 μM) before and after induction of augmentation, it was found that induced responses were not changed after induction of augmentation (103±1%, n=7). Together, these data exclude any postsynaptic contribution to the augmentation seen in Munc13-2 dependent neurons.

The vesicle supply/consumption balance was quantified by examining augmentation as a function of train frequency (1-20 Hz). At all stimulation frequencies, Munc13-1 dominated wild type neurons showed depression, and the degree of depression increased with the train frequency as expected (FIG. 4E). In Munc13-1 deficient, Munc13-2 driven synapses, however, the degree of augmentation generally increased with train frequency. At a stimulation frequency of 1 Hz, we measured 50% augmentation, and at 20 Hz, augmentation reached 1120±210% (n=7) of the basal amplitude. The lack of any form of depression and almost linear increases of augmentation with vesicular consumption in Munc13-2 driven synapses suggests that, in contrast to the wild type situation, vesicular supply is not the rate limiting step for synaptic release in Munc13-2 driven synapses.

### EXAMPLE 11

### Augmentation in Munc13-2 Dependent Synapses is Mediated by an Increase of the Readily Releasable Vesicle Pool Size and the Vesicular Release Probability

Synaptic depression in wild type neurons has been interpreted as depletion of the readily releasable pool of vesicles because pool sizes and synaptic responses are reduced in parallel during high frequency stimulation. Accordingly, recovery from such depression is thought to reflect refilling of a partially depleted vesicle pool (Rosenmund and Stevens, 1996; Wang and Kaczmarek, 1998). To examine whether the pronounced augmentation in Munc13-2 dependent synapses during high frequency stimulation is caused by an increase in the size of the readily releasable vesicle pool, pool sizes during augmentation was determined. Although augmentation is transient, its decay time constant of approximately 8 s allowed to estimate the pool size during the augmented state by applying hypertonic sucrose solution 2 s after the induction of augmentation (FIG. 12A). Hypertonic sucrose solution releases the entire readily releasable pool of vesicles within 3 seconds in a Ca²⁺ independent manner. The total charge integrated over the transient part of the inward current induced by the hypertonic solution represents the size of the readily releasable vesicle pool (Rosenmund and Stevens, 1996). Knowing the average charge produced by a single mEPSC, the number of vesicles in the readily releasable vesicle pool (from all autapses of this neuron) can be determined. For non augmented Munc13-2 dependent synapses, the total pool size summed over all autapses of a neuron was 275±34 vesicles. In contrast, 2 s following a 10 Hz/7 s action potential train, the size of the readily releasable vesicle pool summed over all autapses of a given neuron had increased by 265±40% to 730±110 vesicles (n=12; FIG. 12B, bottom). Under the same conditions, wild type neurons showed a 25±4% decrease in pool size from 3120±450 vesicles before the 10 Hz train to 2355±340 after the train (n=10). Thus, in Munc13-1 deficient, Munc13-2 dependent neurons, the ratio between the numbers of readily releasable vesicles available after and before a 10 Hz stimulation train is 3.5 fold larger than in wild type neurons (FIG. 12B, bottom). Parallel measurements of synaptic EPSC amplitudes before and after a 10 Hz train showed an increase of 600±71% in Munc13-2 dependent synapses (n=35; FIG. 12B, top), which is larger than the increase observed for the pool sizes (265±40%; FIG. 12B, bottom). On the other hand, evoked amplitudes of wild type neurons decreased by 41±8% (n=7; FIG. 5B, bottom). These data demonstrate that high frequency stimulation of Munc13-1 deficient, Munc13-2 dependent synapses primes a pool of vesicles that is reluctant to be primed at low stimulation frequencies. In addition, synaptic responses from Munc13-2 dependent synapses augment more strongly than the pool size itself (600±71 vs. 265±40%), demonstrating that the vesicular release probability immediately after induction of augmentation is also increased approximately twofold, most likely due to the elevated intraterminal Ca²⁺ concentration and thus representing a facilitation component.

So far, it was assumed that our electrophysiological analysis of Munc13-1 deficient, Munc13-2 dependent synapses indicated that the augmentation following high frequency stimulation is due to increases in pool size and release probability. However, it is also possible that high frequency stimulation leads to a ‘reawakening’ of previously silent synapses. To examine whether such reawakening occurs, NMDA receptors of active synapses in Munc13-1 deficient, Munc13-2 dependent neurons at low stimulation frequencies were irreversibly blocked using pharmacological means and subsequently tested for a transient appearance of a new synapse population following a 10 Hz train. EPSCs were stimulated in the presence of 10 μM glycine and 2.7 mM Ca²⁺ but in the absence of Mg²⁺. The slow NMDA receptor component of EPSCs can be blocked specifically, completely, and irreversibly by repeated stimulation of synaptic release in the presence of the open NMDA receptor channel blocker MK-801. Consequently, an induction of augmentation via synapse reawakening should induce a large de novo NMDA EPSC component if a population of previously silent synapses reawakened during the augmentation-inducing 10 Hz train. The NMDA component of EPSCs was blocked by evoking 120 NMDA EPSCs in the presence of 5 μM MK-801. This led to a reduction of the NMDA EPSC component by more than 90%. Following washout of MK-801, the synaptic amplitudes of both AMPA (fast component) and NMDA components (slow component) of EPSCs were monitored before and after a 10 Hz train (7 s). Following a 10 Hz train, the AMPA mediated component of EPSCs transiently increased as expected (n=6, FIGS. 13A-13C). However, apart from the expected augmentation of the minute remaining NMDA component, a new substantial NMDA mediated component did not appear (FIGS. 13B and 13C). This demonstrates (1) that the synapses mediating the augmentation in Munc13-1 deficient, Munc13-2 driven neurons are the same synapses that are active at low stimulation frequencies, (2) that augmentation is restricted to the Munc13-2 dependent synapse population, and (3) that synapse reawakening does not contribute to the observed augmentation in Munc13-1 deficient cells.

### EXAMPLE 12

### Increase of Pool Size and Release Probability During Action Potential Trains is Triggered by Elevation of the Intracellular Ca²⁺ Concentration

High action potential frequencies lead to an increase of the basal intraterminal Ca²⁺ concentration by several hundred nM (Helmchen, 1996). This rise in the Ca²⁺ concentration may be an important factor in short term plastic changes of transmitter release. Consequently, both phenomena contributing to the augmentation observed in Munc13-1 deficient, Munc13-2 dependent synapses following high frequency stimulation, i.e. increases of readily releasable pool size and vesicular release probability, may be mediated by elevated levels of the intraterminal Ca²⁺ concentration. In this case, similar degrees of augmentation should be observed following increases of the intraterminal Ca²⁺ concentration due to manipulations that do not involve the induction of action potentials.

The intraterminal Ca²⁺ concentration independently of action potentials was elevated by external application of short K⁺ pulses (65-150 mM, 0.025-0.8 s) in the presence of 4 mM external Ca²⁺. This K⁺ application should lead to a depolarization of unclamped terminals and to an opening of presynaptic voltage-dependent Ca²⁺ channels, which in turn would result in an increase of the intraterminal Ca²⁺ concentration and augmentation. First the dose dependency and the time course of augmentation was examined in response to K⁺ induced depolarizations. Short pulses (25-800 ms) of isotonic K⁺ (150 mM) in the presence of 4 mM Ca²⁺ were applied to Munc13-1 deficient, Munc13-2 dependent neurons, and synaptic amplitudes were monitored at 1 Hz resolution (FIG. 14). Particularly at short pulse duration (25-100 ms), augmentation developed within the first few synaptic responses, suggesting that Ca²⁺ is able to transform reluctantly primed vesicles into fusion competent vesicles within a few seconds. After longer lasting depolarizations (200-800 ms) the degree of augmentation was smaller and its time course slowed down, presumably due to significant depletion of vesicles from the readily releasable pool during the depolarization and longer lasting and larger Ca²⁺ elevations in the terminal. These data suggests that the Ca²⁺ dependent process underlying augmentation develops very rapidly (1-2 s), making an involvement of certain biochemical effects such as phosphorylation less likely.

To quantify the degree of EPSC and pool augmentation, the briefest K⁺ pulse that caused maximal augmentation (150 mM external K⁺ for 100 ms) was chosen and compared EPSCs and sucrose responses before and after the K⁺ pulse. Both, synaptic responses (603±85%, n=32) and the size of hypertonically induced responses (312±41%, n=6) increased dramatically upon K⁺ stimulation (FIGS. 12A and 12C). In a given cell, K⁺ induced augmentation correlated directly with the augmentation seen after 10 Hz trains of action potentials (EPSC amplitude: 587±53%, n=12; pool size: 262±43%, n=5; see also FIG. 12). In contrast to Munc13-1 deficient, Munc13-2 dependent cells, wild type neurons showed depression of both, synaptic amplitude (to 57±12%, n=14) and pool size (to 64±6%, n=14, not shown) following the K⁺ pulse, again closely resembling the measurements obtained with trains of action potentials (FIG. 12). Above data demonstrate that K⁺ pulses mimic the effects of high frequency stimulation with respect to augmentation induced in Munc13-1 deficient, Munc13-2 dependent synapses, suggesting that in both cases, Ca⁺⁺ elevation in Munc13-1 deficient synapses is responsible for the observed augmentation. To test this further, the stimulation frequency dependence of augmentation in Munc13-1 deficient, Munc13-2 synapses was compared under standard conditions with cells that were pretreated with the membrane permeable derivative of the Ca²⁺ chelator EGTA, EGTA-AM (50 μM, 20 min). This treatment should increase the Ca²⁺ buffering capacity in presynaptic terminals significantly and therefore lower the intraterminal Ca²⁺ elevations during action potential trains. Although not quantified, the synaptic EPSC amplitudes at low frequency stimulation rates were smaller in EGTA pretreated cells as compared to untreated cultures, indicating the presence of significant EGTA concentrations in presynaptic terminals. As shown in the frequency-augmentation profile (FIG. 14D), augmentation was strongly reduced in the EGTA-AM pretreated cells at frequencies below 40 Hz (p<0.02 at all frequencies tested, except 100 Hz). At 10 Hz, untreated Munc13-2 dependent cells augmented 6.17±0.51 fold (n=29), while EGTA-AM treated cells augmented only 2.0±0.4 fold (n=8, p<0.0002). Augmentation in untreated Munc13-2 dependent cells reached a maximum (>10 fold) at around 20 Hz, while at 40 and 100 Hz, augmentation was submaximal (5-7 fold), which correlates nicely with the submaximal augmentation induced by K⁺ pulses with a duration above 200 ms (FIG. 14B). EGTA-treated Munc13-2 dependent cells reached maximal augmentation (11.0±1.5 fold, n=8) at a higher frequency (40 Hz) than untreated cells but showed a similar degree of augmentation at 100 Hz, further demonstrating that, presumably due to buffer saturation, higher train frequencies are needed to reach full augmentation.

The data on the effects of EGTA-AM support the conclusion that Ca²⁺ elevation in Munc13-1 deficient, Munc13-2 dependent synapses is responsible for the observed augmentation. This leads to the question as to whether the function of Munc13-1 deficient synapses is changed at the final Ca²⁺ triggered step of exocytosis. If so, this can be shown by examining the Ca²⁺ dependency of evoked release at low stimulation frequencies. This was studied by measuring EPSCs in the presence of systematically varying external Ca²⁺ concentrations. Application of an external solution containing 4 mM Ca²⁺ and 4 mM Mg²⁺ was alternated with application of external solutions containing lower or higher Ca²⁺ concentrations and a constant Mg²⁺ concentration. EPSCs at varying Ca²⁺ concentrations were measured and normalized to the response seen with the standard external solution containing 4 mM Ca²⁺ and 4 mM Mg²⁺. No evidence for a difference in Ca²⁺ dependency of release between wild type (n=6-32) and Munc13-1 deficient, Munc13-2 dependent synapses (n=11-43, not shown) was found. Thus the last, Ca²⁺ triggered fusion step in synaptic transmitter release is not the process that is differentially regulated by Munc13 isoforms.

### EXAMPLE 13

### Ca²⁺ Induced Augmentation is Not Caused by Altered Function of Kinases, Phosphatases or the Actin Cytoskeleton

In neuroendocrine cells and hippocampal neurons, concomitant action of PKC and Ca²⁺ can increase the size of the readily releasable vesicle pool (Gillis, 1996; Stevens, 1998; von Ruden, 1993). Pharmacological tools were used to examine whether Ca²⁺ mediates augmentation in Munc13-2 dependent synapses via Ca²⁺ dependent kinases or phosphatases or through modifications of the actin cytoskeleton (FIG. 8A). Application of the PKCγ blocker Gö-6973 (100 nM) or of the (at this concentration) more unspecific kinase blocker staurosporine (100 nM) failed to suppress augmentation. Likewise, a cocktail of phosphatase inhibitors containing cypermethrin (1 nM), calyculin A (10 μM), and cyclosporine A (1 μM), or okadaic acid (1 μM) alone did not alter augmentation. Similar results were obtained with forskoline treatment (1 μM) which leads to increased intracellular CAMP levels. Moreover, the membrane permeable blocker of actin polymerization, latrunculin B (10 μM), also failed to affect augmentation. Munc13-1 contains a diacylglycerol/β-phorbol ester binding C1 domain that is involved in the upregulation of synaptic transmission in Xenopus neuromuscular junctions (Betz, 1998) and is responsible for the fast β-phorbol ester induced synaptic potentiation in hippocampal neurons as well as for the maintenance of synaptic responses during trains of action potentials. In view of the data presented in the present study, observations on the functional importance of Munc13 C1 domains raise the possibility that functional differences between Munc13-1 and Munc13-2 with respect to their role in short term plasticity are caused by differences in a C1 domain induced signal cascade. The involvement of the C1 domain as tested by inhibiting the production of the ligand to the C1 domain, diacylglycerol, using the specific PLC inhibitor U73223. Preincubation of U73223 at 3 μM for 150 seconds led to an almost complete inhibition of augmentation, while leaving basal release essentially unaffected (FIG. 15A). The inactive analogue U73445 (3 μM) however failed to modify augmentation. Together, these data indicate that the augmentation observed in Munc13-2 dependent synapses following high frequency stimulation is not due to Ca²⁺ dependent changes in the function of the actin cytoskeleton or in phosphorylation or dephosphorylation reactions that may modify proteins relevant for vesicle priming or release. Rather, phospholipase C activity and the presence of diacylglycerol is required for induction of augmentation, presumably acting on the C1 domain of Munc13s.

### EXAMPLE 14

### Munc13 Isoform Specific Differential Potentiation of Transmitter Release by β-Phorbol Esters Correlates with Differential Augmentation

A stable analogue of Diacylglycerol, the β-phorbol esters are known to activate protein kinase C and to bind to the C1 domain of Munc13-1 (Betz, 1998). Given the above requirement of diacylglycerol for induction of augmentation suggests that augmentation is linked to the activation of the C1 domain, it was examined whether the β-phorbol ester sensitivity of Munc13-1 and Munc13-2 dependent synapses differ in a similar manner as do their facilitation/augmentation properties. It was found that β-phorbol ester induced EPSC potentiation in Munc13-1 deficient, Munc13-2 dependent cells was much larger than that seen in Munc13-1 dominated wild type synapses. Synaptic potentiation upon application of PDBU (100-1000 nM) was 570±118% (n=12) in Munc13-2 dependent synapses and 199±25% (n=7) in Munc13-1 dominated wild type synapses (FIG. 15B). Furthermore, β-phobol ester induced potentiation and train induced augmentation were not additive (FIG. 15C). Frequency-augmentation profiles from Munc13-2 dependent cells in the absence or presence of 1 μM PDBU showed that at frequencies at or above 20 Hz, untreated Munc13-2 dependent synapses and PDBU treated synapses reached similar degrees of augmentation. β-Phorbol ester induced potentiation of IPSCs was much less pronounced and differences between Munc13-1 and Munc13-2 dominated cells were very small (FIG. 15B). Thus, β-phorbol ester induced potentiation of transmitter release in Munc13-2 dependent synapses parallels train induced augmentation.

To examine whether the pronounced β-phorbol ester induced EPSC increase in Munc13-2 dependent cells is due to an underlying increase in readily releasable vesicle pool, changes in pool size in response to PDBU were examind (see also Stevens and Sullivan, 1998). The readily releasable vesicle pool increased by 30±15% (n=5) in Munc13-2 dependent synapses and by 11±5% in Munc13-1 dominated wild type synapses (n=4; FIG. 15B). Thus, the strongly potentiating effects of β-phorbol esters on evoked transmitter release from Munc13-2 dependent cells is not caused by a corresponding increase in the readily relesable vesicle pool size. This is in striking contrast to augmentation induced by high frequency action potential trains, where pool size and evoked EPSCs are increased in parallel. Although the potentiating effects of β-phorbol esters on EPSC amplitudes and the train induced augmentation were not additive in Munc13-2 dependent cells, these findings suggest that β-phorbol esters exert their effects through a mechanism that is only partially redundant with the mechanism of Ca²⁺ induced augmentation.

To confirm that the different degree of β-phorbol ester induced potentiation is strictly dependent on the respective Munc13 isoform expressed, Semliki Forest Virus rescue experiments were carried out in Munc13-1/Munc13-2 double deficient neurons. For this purpose, the effects of PDBU on evoked EPSCs were examined in Munc13-1/Munc13-2 double deficient neurons that had been rescued by virus mediated overexpression of either Munc13-1 or ubMunc13-2. In neurons rescued with ubMunc13-2, PDBU application caused an EPSC potentiation of 510±83% (n=11). In contrast, EPSCs in neurons rescued with Munc13-1 were potentiated by only 247±22% (n=12, FIG. 15D). These findings clearly demonstrate that the degree to which β-phorbol esters potentiate transmitter release is indeed dependent on the respective Munc13 isoform expressed, as is the case with train induced augmentation.

In general, synaptic transmission is highly dependent on the recent history of presynaptic activity. During or following periods of stimulation, particularly at higher frequencies, most synapses show depression due to readily releasable vesicle pool depletion and, in many cases, due to a decrease of the vesicular release probability. However, forms of enhanced synaptic transmission following previous activation also exist. They often depend on the pattern of preceding activity and vary mainly in the time course of the enhancement. Depending on their time course and duration, these forms of synaptic enhancement are termed facilitation, augmentation, or potentiation (Zucker, 1989).

The analysis of use dependent synaptic enhancement is complicated because facilitation and augmentation are often masked by simultaneous depression (Magleby, 1987; Zucker, 1999). Enhancement of synaptic transmission can be determined by multi parameter fitting (Clements, 2000; Varela, 1997), or after artificial reduction of the release probability, e.g. by lowering the external Ca²⁺ concentration (Stevens and Wesseling, 1999), by replacing Ca²⁺ with Sr⁺ or Ba²⁺ (Magleby, 1987), by activating presynaptic inhibitory neurotransmitter receptors (Clements and Silver, 2000; Kreitzer and Regehr, 2000), or by other pharmacological manipulations (Fisher, 1997).

In the present study, a large and usually dominant population of depressing synapses was silenced by genetically abolishing the expression of Munc13-1. This genetic manipulation unmasked a Munc13-2 dependent synapse population whose release properties at low frequency stimulation are indistinguishable from wild type, but in which one form of short term plasticity, i.e. augmentation, dominates over other forms of plasticity. As observed in other systems, this augmentation develops within 300-1000 ms, is characterized by an up to 10 fold enhancement of synaptic transmission, decays with a time constant of approximately 8 s (FIG. 9), and is strictly dependent on Ca²⁺ influx (FIG. 14) (Zucker, 1989). Assuming that this type of Munc13-2 dependent synapse is not only present in our culture system but also relevant in situ, it was used herein as a model for augmentation and determined its characteristics.

The functional analysis provided of Munc13-2 dependent synapses shows that augmentation is a consequence of both, an increase of the readily releasable vesicle pool size and of the vesicular release probability (FIG. 12). Most likely, the underlying molecular mechanism of augmentation only involves a strong enhancement of Munc13-2 mediated priming activity in response to Ca²⁺, leading to the observed increase of the readily releasable vesicle pool. The increase in vesicular release probability, on the other hand, can simply be explained by the facilitating action of elevated background Ca²⁺ concentrations on the release trigger. In contrast, previous studies in hippocampal cultures (Stevens and Wesseling, 1999) and other systems (Zucker, 1999) identified only increased vesicular release probabilities or pool refilling rates but no changes in the releasable vesicle pool size as causes for activity dependent changes in synaptic efficacy. The dramatic increase of the readily releasable vesicle pool in Munc13-2 dependent synapses (250%) is an unprecedented type of synaptic enhancement in central neurons which likely was overseen due to the small size of this synapse population. An interesting parallel is provided by chromaffin cells in which priming occurs in the presence of only very low Munc13-1 levels (Ashery, 2000) and the steady state releasable vesicle pool size is tightly regulated by Ca²⁺ in conjunction with PKC (Heinemann, 1993; Smith, 1998; Stevens, 1998; von Rüden, 1993).

As mentioned above, a rise in the intracellular Ca²⁺ concentration is thought to be an important contributing factor in use dependent synaptic enhancement. However, the synaptic target of Ca²⁺ action in all studied forms of short term plasticity have remained enigmatic. Here, it is demonstratd that both, action potential trains at high frequency (FIG. 11) and K⁺ induced depolarization (FIG. 14) lead to strong augmentation in Munc13-2 dependent synapses, suggesting that influx of Ca²⁺ is the triggering signal. This is supported by the effects of EGTA-AM on augmentation in these cells (FIG. 14D).

Because no evidence for a difference in Ca²⁺ dependency of release was found between Munc13-1 dependent and Munc13-2 dependent synapses, components of the Ca²⁺ triggered release machinery are unlikely to involved in the augmentation seen in Munc13-2 dependent cells. In view of the fact that application of high concentrations of inhibitors of kinases and phosphatases do not influence augmentation (FIG. 15A), enzymes involved in protein phosphorylation and dephosphorylation are unlikely mediators of the Ca²⁺ induced augmentation which was observed. Likewise, processes involving the regulation of vesicle mobility through modifications of cytoskeleton structure or function (Krucker 2000; Ryan, 1999) do not seem to contribute to the Ca²⁺ induced augmentation observed herein (FIG. 15A). Rather, the finding that the typical depressing/Munc13-1 vs. augmenting/Munc13-2 synapse phenotype can be reconstituted shortly after virus mediated rescue of Munc13-1/Munc13-2 double deficient neurons with the respective isoforms (FIGS. 11C and 11D) suggests that the differences between the two synapse populations are caused by their differential equipment with Munc13 isoforms. Munc13-2 itself would then be the target of Ca²⁺ that responds to increases of intraterminal Ca²⁺ concentrations with a strongly enhanced priming rate and thereby causes the observed augmentation in Munc13-2 dependent synapses.

How could Munc13-1 and Munc13-2 act as Ca²⁺ sensors that differentially affect presynaptic short term plasticity? Both proteins contain a central C2 domain that exhibits all essential structural prerequisites for Ca²⁺ binding (Brose, 1995). However, Ca²⁺ binding to recombinant Munc13-1 or Munc13-2 C2 domains was never detected biochemically ((Brose, 1995); A. Betz and N. B., unpublished results). Nevertheless, direct binding of Ca²⁺ by Munc13-1 or Munc13-2 in vivo may affect their priming activity. Alternatively, Ca²⁺ may regulate Munc13 isoforms indirectly. One such mechanism may involve Ca²⁺/calmodulin binding to Munc13s. However, the calmodulin binding site that was initially discovered in Drosophila Unc-13 (Xu, 1998) is only conserved in Munc13-1 and absent in b/ubMunc 13-2 (H. Junge, A. Betz and N. B., unpublished results). A second, more likely mechanism may involve diacylglycerol mediated activation of Munc13s. All Unc-13/Munc13 like proteins contain a conserved C1 domain that binds diacylglycerol and its β-phorbol ester analogues. The present study shows that β-phorbol ester dependent potentiation of EPSCs mirrored the degree of train induced augmentation (compare FIGS. 11 and 15). It is furthermore demonstrated that inhibition of phospholipase C prevents the induction of augmentation (FIG. 15). In addition, experiments on mouse mutants in which the β-phorbol ester binding site of Munc13-1 was disabled by homologous recombination (Munc13-1^{H567K}/Munc13-1^{H567K}) support the view of a role of Munc13 C1 domains in activity dependent refilling of readily releasable vesicle pools (Rhee, 2001). On the basis of these findings, the Ca²⁺ dependency of the enhanced pool refilling kinetics that underlies the augmentation in Munc13-2 dependent synapses would then be the result of increased synaptic diacylglycerol levels following a Ca²⁺ dependent activation of phospholipase C. Similar to its role in PKC, the Munc13 C1 domains would thus be ideally suited to respond to membrane proximal and activity induced increases of presynaptic Ca²⁺/diacylglycerol concentrations with enhancement of its priming activity. The differences in short term plasticity of distinct synapses formed by a single excitatory axon are not due to generally different release mechanisms. Rather, the differences arise from the selective expression of Munc13 isoforms which themselves differ with respect to Ca²⁺/diacylglycerol dependent priming activity.

An increasing number of studies on principle functions of synapses appreciates the heterogeneity of synaptic function (Craig and Boudin, 2001). Synapses are not stereotypic translators of action potentials into neurotransmitter release. Rather, they vary with respect to size, postsynaptic sensitivity, equipment with modulatory signal transduction cascades, release probability, and dynamics of release over a wide range of presynaptic action potential frequencies.

Here it is shown that the differential localization of Munc13s controls release dynamics of individual synapses. In vivo, such differences between synapses could be relevant in several different physiological contexts. First, different types of synapses formed by the same axon may allow a given nerve cell to transmit information of different quality in a target cell dependent manner. In this scenario, different types of target cells (e.g principal and interneurons within a brain nucleus) would provide different targeting signals for Munc13 isoforms. Second, a balanced mix of facilitating and depressing synapses formed between two neurons by a single axon will broaden the bandwidth within which action potentials can be faithfully transduced into transmitter release. Finally, transforming depressing synapses into augmenting ones or vice versa by simply exchanging the Munc13 isoform represents an important mechanism to induce lasting changes in synaptic efficacy, regardless of whether they follow developmental, learning or even pathophysiological processes.

### EXAMPLE 15

### Basic Characterization of Mice with a H567K Point Mutation in the Munc13-1 Gene

Mice with a H567K point mutation in the Munc13-1 gene, which abolishes β-PE binding by Munc13-1, were generated as described in Experimental Procedures (FIG. 2). Like Munc13-1 deletion mutants and homozygous Munc13-1^{H567K}/Neo (m_{Neo}/m_{Neo}) mice, homozygous mutant/Cre-recombined Munc13-1^{H567K}/Munc13-1^{H567K} mice (m/m) were not viable. However, they expressed Munc13-1^{H567K} at levels comparable to those of Munc13-1 in wild type littermates (FIG. 2E). This demonstrates that intronic insertion of the remaining loxP site does not interfere with mRNA synthesis, splicing or stability, and that the H567K mutation does not lead to major misfolding or destabilization of Munc13-1 protein (see also Betz, 1998). Moreover, the ratio of soluble to membrane bound Munc13-1 was not altered in Munc13-1^{H567K}/Munc13-1^{H567K} mutants (FIG. 2E), indicating that diacylglycerol binding by Munc13-1 is not responsible for its tight association with the presynatic active zone.

Immediately after birth, Munc13-1^{H567K}/Munc13-1^{H567K} mutants are indistinguishable from wildtype control animals. They breathe, react to tactile stimuli, and exhibit pain avoidance reflexes. However, like Munc13-1 deletion mutants homozygous Munc13-1^{H567K}/Munc13-1^{H567K} mutant mice stop breathing and die within 2-3 hours after birth, suggesting that a general brain stem malfunction causes death. The overall structure and cytoarchitecture of brains and the distribution of the synaptic marker synaptophysin in Munc13-1^{H567K}/Munc13-1^{H567K} mutants were found to be indistinguishable from wild type littermates (not shown). Heterozygous Munc13-1^{H567K} mutants were essentially identical to wild type controls in these and all following analyses (not shown), demonstrating that the Munc13-1^{H567K} protein has no dominant negative effect.

### EXAMPLE 16

### Spontaneous and Evoked Synaptic Transmission in Munc13-1^{H567K}/Munc13-1^{H567K} Mutants

To examine how destruction of the diacylglycerol/□-PE binding site in Munc13-1^{H567K}/Munc13-1^{H567K} mutants affects synaptic transmission and ultimately results in the lethal phenotype, we performed electrophysiological analyses of individual hippocampal neurons in microisland cultures, which form recurrent ‘autaptic’ synapses onto themselves. In this culture preparation, spontaneous transmitter release, release of the readily releasable vesicle pool induced by hypertonic solutions, and action potential evoked release arise from the same synapse population and can be measured readily with patch clamp techniques. The analysis concentrated on glutamatergic neurons because Munc13-1 function is essential for these but not for GABAergic cells in hippocampal primary cultures (Augustin, 1999b).

Routinely, cells from Munc13-1^{H567K}/Munc13-1^{H567K} mutants were analyzed 12-20 day after plating and compared to wild type control cells obtained from littermates. In some experiments we employed Munc13-1/Munc13-2 double deficient neurons as model cells to study the characteristics of the Munc13-1^{H567K} variant in comparison to wild type Munc13-1 following overexpression using the Semliki-Forest-Virus system. In contrast to Munc13-1 deficient glutamatergic hippocampal neurons, which still show measurable glutamate release (10% of wild type levels; Augustin, 1999b), double homozygous Munc13-1/Munc13-2 deletion mutant neurons are characterized by a complete lack of spontaneous and evoked transmitter release but can be rescued by reintroduction of Munc13 proteins. This rescue approach was persued in Munc13-1/Munc13-2 double mutant neurons instead of breeding the Munc13-1^{H567K}/Munc 3-1^{H567K} mutation into the Munc13-2 deletion mutant background because it was much faster and allowed the direct comparison of additional mutant (Betz, 2001) and wild type Munc13-1 variants in the same cultures. Autaptic responses were obtained after brief (1-2 ms) somatic depolarization. This induces an unclamped action potential that is followed by a postsynaptic response with a delay of 2-4 ms. Excitatory, glutamatergic synaptic currents were identified by their characteristic pharmacological and kinetic properties.

Although the perinatally lethal phenotype of Munc13-1^{H567K}/Munc13-1^{H567K} mice is similar to that of Munc13-1 deletion mutants, their electrophysiological characteristics were found to be strikingly different. In contrast to Munc13-1-deficient neurons, Munc13-1^{H567K}/Munc13-1^{H567K} cells produced robust evoked EPSC amplitudes that were only slightly smaller than but not statistically different from wild type values (4.87±0.45 nA, n=91, Munc13-1^{H567K}/Munc13-1^{H567K} mutant; 5.8±0.7 nA, n=83, wild type) (FIG. 16A). Likewise, mean frequency (3.7±1.3 Hz, n=10, Munc13-1^{H567K}/Munc13-1^{H567K} mutant; 4.2±1.9 Hz, n=12, wild type), amplitude (23.2±1.4 pA, n=10, Munc13-1^{H567K}/Munc13-1^{H567K} mutant; 24.5±1.3 pA, n=12, wild type) and charge (92±7 pC, n=10, Munc13-1^{H567K}/Munc13-1^{H567K} mutant; 96±4 fC, n=12, wild type) of spontaneous responses were not different between Munc13-1^{H567K}/Munc13-1^{H567K} and wild type neurons.

In a separate set of experiments, it was found that evoked EPSC amplitudes from Munc13-2 deficient cells that express Munc13-1 as the only Munc13 isoform (1.32±0.27 nA, n=17) were indistinguishable from responses measured in Munc13-1/Munc13-2 double deficient neurons overexpressing Munc13-1^{H567K} (1.51±0.3 nA, n=21) (FIG. 16B). These data demonstrate that inactivation of the diacylglycerol/β-PE binding site in Munc13-1 leaves the postsynaptic responsiveness of neurons unaffected and does not abolish priming of synaptic vesicles or inhibit their evoked release (see also Ashery, 2000).

### EXAMPLE 17

### Effects of β-PEs on Munc13-1^{H567K}/Munc13-1^{H567K} Neurons

Given that spontaneous and evoked responses in Munc13-1^{H567K}/Munc13-1^{H567K} mutants were normal, consequences of the Munc13-1^{H567K} mutation for β-PE mediated effects on synaptic transmission was next examined. Application of 1 μM β-PE dibutyrat (PDBU) to wildtype neurons for one minute led to a rapid (8 s time constant) and robust enhancement of evoked EPSC amplitudes (191±17% of baseline values, n=30). Following removal of PDBU, the synaptic amplitude returned to baseline within 3-4 minutes (27 s time constant; FIG. 16C). In contrast, Munc13-1^{H567K}/Munc13-1^{H567K} neurons showed a strongly reduced sensitivity towards β-PE with application of 1 μM (PDBU) resulting in a slower (16 s time constant) and very moderate increase in evoked EPSC amplitudes (131±7% of baseline values, n=34, p<0.001 compared to wild type control cells; FIG. 16D). Control experiments showed that α-PEs, inactive analogues of the respective β forms that do not bind to C₁ domains, had no effect on evoked EPSCs in both, wild type (n=9) and Munc13-1^{H567K}/Munc13-1^{H567K} neurons (n=12). In addition to evoked responses, the effect of 1 μM PDBU on mEPSC frequency in control and Munc13-1^{H567K}/Munc13-1^{H567K} neurons was also examined because numerous reports showed β-PE dependent increases in mEPSC frequency. It was found that in wild type neurons, the mEPSC frequency increased to 239±34% (n=8) of control levels while in Munc13-1^{H567K}/Munc13-1^{H567K} cells a much weaker increase was observed (127±16% of control levels, n=7). These data provide the first direct evidence suggesting that Munc13-1, and not PKCs, is the main presynaptic target for β-PE mediated enhancement of synaptic transmitter release.

Although dramatically reduced in comparison to the wild type situation, β-PE mediated enhancement of spontaneous and evoked synaptic transmitter release was still readily detectable in Munc13-1^{H567K}/Munc13-1^{H567K} neurons (FIG. 16D). To test whether this is due to the presence of Munc13-2, the only Munc13 isoform coexpressed with Munc13-1 in glutamatergic hippocampal nerve cells (Augustin, 1999a; 2001), wild type Munc13-1 or Munc13-1^{H567K} was overexpressed in Munc13-1/Munc13-2 double deficient neurons (Brose, 2001) using the Semliki-Forest-Virus system and tested their sensitivity to β-PEs. Similar to wild type neurons, Munc13-1/Munc13-2 double deficient neurons overexpressing wild type Munc13-1 responded to application of 1 μM PDBU with a rapid and robust increase in evoked EPSCs (246±22% of control levels, n=12; FIGS. 16E and 16F). In contrast, Munc13-1/Munc13-2 double deficient cells overexpressing Munc13-1^{H567K} were completely insensitive to 1 μM PDBU (108±5% of control levels, n=17; FIGS. 16E and 16F) although evoked EPSC amplitudes where reconstituted to control levels (FIG. 16B). Together, these data demonstrate that β-PE dependent enhancement of presynaptic transmitter release from hippocampal neurons is entirely dependent on the presence of Munc13-1 and Munc13-2.

All results shown herein are in clear conflict with the widely held view that PKCs are the main β-PE targets in the presynapse. Interestingly, published conclusions about the role of PKCs in the regulation of transmitter release are almost exclusively based on indirect pharmacological evidence. Data from the genetic approach presented here, however, suggest that Munc13s and not PKCs are responsible for the β-PE mediated enhancement of neurotransmitter release. This view was further supported in a more detailed pharmacological analysis of wild type hippocampal neurons. Here, it was surprisingly found that the specific PKC inhibitor GÖ 6983, a bisindolylmaleimide derivative that is thought to specifically interact with ATP binding sites of several PKCs, had no effect on the β-PE induced increase in evoked EPSCs (160±9% of baseline values, n=10, with 1 μM PDBU alone; 150±6% of baseline values, n=10, after preincubation for 2 min and in the presence of 3 μM GÖ 6983). This demonstrates that GÖ 6983 sensitive PKCs are not involved in the β-PE effects observed here. In addition, the effects of bisindolylmaleimide I (GÖ 6850), a widely used but somewhat less specific inhibitor of PKCs were examined. For that purpose, wild type neurons were first treated for 1 min with 1 μM PDBU and the induced increase of evoked EPSC amplitudes (90±19% increase over baseline value of 3.06±0.56 nA, n=6; FIG. 17A) was measured. Following washout of PDBU, application of bisindolylmaleimide I (3 μM) led to a partial and irreversible rundown of evoked EPSC amplitudes, with a 41±11% reduction of responses (n=6) over a four minute application period (FIGS. 17B and 17C). However, the resulting reduced evoked EPSC responses were still potentiated by application of 1 μM PDBU (FIG. 17B), and the degree of this potentiation was similar to the one observed with PDBU alone (73±8.3%, n=6; FIG. 17D). Munc13-1^{H567K}/Munc13-1^{H567K} neurons showed a very similar bisindolylmaleimide 1 dependent reduction of evoked EPSC amplitudes (not shown), demonstrating that this rundown is not directly related to the function of the Munc13-1 C₁ domain. Bisindolylmaleimide 1 acts upon evoked EPSC responses through an unknown, possibly toxic mechanism but does not prevent the β-PE induced potentiation of transmitter release, again strongly supporting the result that PKCs are not involved.

Most published PKC studies depended entirely on pharmacological tools whose specificity is still poorly characterized. Indeed, it was found here that bisindolylmaleimide I/GÖ 6850, one of the most frequently used ‘PKC-specific’ drugs, inhibits neurotransmission even in the absence of β-PEs (FIG. 15), suggesting that the drug has a nonspecific toxic effect on nerve cells that compromises its use as a pharmacological tool. Moreover, an alternative ‘specific’ PKC inhibitor, GÖ 6983, had no effect on the β-PE induced increase in transmitter secretion studied here (not shown). Thus, published studies using pharmacological tools to examine the role of PKCs in the regulation of transmitter release have to be interpreted with great care. Interestingly, the only case where an alternative approach was taken (genetic deletion of PKCγ in mice; Goda, 1996) gave no evidence for an involvement of PKCs in the regulation of transmitter release. Even if possible problems with the specificity of pharmacological tools in the analysis of PKCs are put asite, several published studies indicated the presence of alternative functional β-PE receptors in the presynapse because effects of the drug were not completely reversed by ‘specific’ PKC inhibitors (Stevens and Sullivan, 1998; Honda, 2000; Hori, 1999; Redman, 1997; Iwasaki, 2000; Waters, 2000). In the most striking example (Iwasaki, 2000), β-PE mediated effects in PC12 cells on protein phosphorylation by PKCs and secretion were shown to be completely distinct with respect to time course, dependence on β-PE concentration, and sensitivity to PKC inhibitors and less specific kinase blockers. So far, the present data shown herein above and below allow firm conclusions about the relative importance of Munc13s and PKCs in the control of neurotransmitter release. However, at least ubMunc13-2 is also expressed in nonneuronal tissues (Song, 1998; Betz, 2001) and may be involved in physiological responses of kidney cells to hyperglycemia (Song, 1998). In view of the present study, the problems with PKC ‘specific’ pharmacological tools mentioned above, and the ubiquitous expression of certain Munc13 isoforms, a critical reevaluation of allegedly PKC mediated effects in nonneuronal tissues is in place.

### EXAMPLE 18

### Expression and Function of PKCs in Munc13-1^{H567K}/Munc13-1^{H567K} Neurons

Although highly suggestive of a PKC independent function of Munc13s in mediating presynaptic β-PE effects, the data presented so far are equally compatible with the possibility that the H567K point mutation in Munc13-1 influences levels or function of PKCs. Thus, the Munc13-1^{H567K} variant could compromise expression or stability of PKCs, resulting in an overall reduction of cellular β-PE responses. Alternatively, Munc13s could have a role as essential regulators or substrates of PKCs that depends on an intact β-PE binding site in Munc13s.

To distinguish between these possibilities, the protein expression levels of PKCs α, β, γ, δ, ε, λ, and τ was first examined by densitometric analysis of ECL signals in Western blots obtained with brain homogenates from wild type and Munc13-1^{H567K}/Munc13-1^{H567K} mice. It was found that the protein levels of all tested PKCs were unaffected by the Munc13-1^{H567K}/Munc13-1^{H567K} mutation (FIGS. 18A and 18B). In a second set of experiments, we examined β-PE dependent PKC activity in cultures from wild type and Munc13-1^{H567K}/Munc13-1^{H567K} mutant brains. For that purpose, cortical/hippocampal primary cultures were loaded with ³²P-orthophosphate and stimulated with 1 μM PDBU. Labeled proteins were separated by 2D-electrophoresis and visualized autoradiographically. Autoradiographs of wild type and Munc13-1^{H567K}/Munc13-1^{H567K} mutant cultures showed identical patterns of proteins that were phosphorylated in a β-PE dependent manner (FIG. 18C), demonstrating that overall β-PE dependent PKC activity is not significantly affected by the Munc13-1^{H567K}/Munc13-1^{H567K} mutation. A third series of experiments concentrated on the β-PE dependent phosphorylation of two identified presynaptic targets of PKCs, SNAP-25 (Shimazaki, 1996) and GAP-43/B-50 (de Graan, 1994). Cultures from wild type and Munc13-1^{H567K}/Munc13-1^{H567K} mutant brains were prepared which were then stimulated with 1 μM PDBU, harvested and analyzed by SDS-PAGE and Western blotting using phosphopeptide specific antibodies (Iwasaki, 2000; Kawakami, 2000). Like in the 2D-electrophoresis experiments described above (FIG. 18C), no differences between the tested genotypes with respect to PKC activity were detected. SNAP-25 and GAP-43 were strongly phosphorylated in a β-PE dependent manner in both, wild type and Munc13-1^{H567K}/Munc13-1^{H567K} mutant cultures (FIG. 18D). This demonstrates that PKC dependent phosphorylation of synaptic targets is normal in Munc13-1^{H567K}/Munc13-1^{H567K} mutants. In a final set of experiments, it was examined whether Munc13-1 acts as a substrate for PKCs, and whether PKC dependent phosphorylation of Munc13-1 is affected by the H567K mutation. To do this, again cortical/hippocampal primary cultures from wild type and Munc13-1^{H567K}/Munc13-1^{H567K} mutant brains were used, which were loaded with ³²P-orthophosphate and stimulated with 1 μM PDBU. Proteins were extracted with detergent and Munc13-1 was immunoprecipitated using a specific polyclonal antibody (Betz, 1997). Phosphorylated and immunoprecipitated material was separated by SDS-PAGE and analyzed by autoradiography. In parallel, aliquots of the same samples were analyzed by Western blotting and densitometric analysis of Munc13-1 specific ECL signals. It was found that Munc13-1 was weakly phosphorylated in a constitutive, β-PE independent manner in all genotypes tested (FIG. 18E). However, neither in wild type nor in Munc13-1^{H567K}/Munc13-1^{H567K} mutant brains a β-PE dependent phosphorylation of Munc13-1 was observed. The ratio between immunoprecipitated and phosphorylated Munc13-1 was very similar between all genotypes and treatment groups (FIG. 18E).

Together with the genetic and electrophysiological evidence described so far, this biochemical examination of PKCs in Munc13-1^{H567K}/Munc13-1^{H567K} neurons provide very strong support for the notion that presynaptic effects of β-PEs and, by deduction, of diacylglycerol on neurotransmitter release are mainly if not exclusively mediated by Munc13s and not by PKCs.

The most striking conclusion of the present study is that Munc13s, and not PKCs, are the only receptors involved in the acute regulation of presynaptic transmitter release by β-PEs or diacylglycerol. This view is based on three key findings: (1) Deletion of the β-PE binding site in Munc13-1 by homologous recombination in embryonic stem cells leads to mutant neurons (Munc13-1^{H567K}/Munc13-1^{H567K}) which exhibit a dramatic reduction in. β-PE sensitivty of transmitter release (FIGS. 16C and 16D). This reduction is complete when the contribution of wild type Munc13-2 in mutant cells is abolished (FIGS. 16E and 16F). (2) Expression and β-E dependent activity of PKCs are normal in Munc13-1^{H567K}/Munc13-1^{H567K} mutant neurons (FIG. 18). Therefore, Munc13-1 is not a regulator of PKCs and the consequences of the Munc13-1^{H567K} mutation are not due to an indirect effect on PKCs. (3) Munc13-1 is not a PKC substrate (FIG. 18) and therefore unlikely to act as a downstream effector of PKCs. Therefore, the consequences of the Munc13-1^{H567K} mutation are not due to the disruption of a hypothetical signaling cascade that is initiated by PKCs and propagated by Munc13s.

The results provided herein are, therefore, in clear and direct conflict with the published view that PKC functions in the control of neurotransmitter release.

### EXAMPLE 19

### Functional Consequences of the Munc13-1^{H567K} Mutation for Synaptic Transmission

In view of the fact that evoked EPSCs in Munc13-1^{H567K}/Munc13-1^{H567K} neurons remained unchanged (FIGS. 16A and 16B) despite the dramatic changes in β-PE sensitivity, the search for functional alterations induced by the H567K mutation was expanded. In order to identify deficits in Munc13-1^{H567K}/Munc13-1^{H567K} neurons that might contribute to the lethal mutant phenotype, it was next examined releasable vesicle pool sizes and release dynamics in Munc13-1^{H567K}/Munc13-1^{H567K} and wild type control cells. In a first set of experiments, the size of the readily releasable vesicle pool as defined was examined by those quanta that are released during the transient burst of exocytotic activity following application of hypertonic sucrose solution (Stevens and Tsujimoto, 1995; Rosenmund and Stevens, 1996). It was found that readily releasable vesicle pools were significantly smaller in Munc13-1^{H567K}/Munc13-1^{H567K} neurons (0.58±0.11 nC, n=81) than those measured in wild type control cells (1.04±0.11 nC, n=72, p<0.001; FIG. 19A). Normalization of these values to mEPSC response sizes in the same cells gave estimates of 6373±660 and 10947±1157 fusion competent vesicles in Munc13-1^{H567K}/Munc13-1^{H567K} and wild type neurons, respectively. However, employing a paired pulse stimulation paradigm with consecutive hypertonic sucrose pulses at variable interpulse intervals, it was determined that the rate by which the readily releasable vesicle pools were refilled after complete depletion was unaffected by the Munc13-1^{H567K}/Munc13-1^{H567K} mutation (FIG. 19B).

Unaltered evoked EPSC amplitudes (FIG. 16A) together with significantly reduced readily releasable vesicle pools (FIG. 19A) indicate an increase in the average vesicular release probability Pᵥᵣ in Munc13-1^{H567K}/Munc13-1^{H567K} mutant cells. Indeed, when Pᵥᵣ was determined by dividing the quantal content of the average evoked EPSC by the pool size, we obtained values of 10.5±0.8% (n=81) for Munc13-1^{H567K}/Munc13-1^{H567K} and 6.9±0.6% (n=72) for wild type cells, respectively (p<0.001, FIG. 19C). Interestingly, similar increases in Pᵥᵣ over wild type control cells were observed in Munc13-1/Munc13-2 double deficient neurons overexpressing Munc13-1^{H567K} (FIG. 19C), suggesting that increased Pᵥᵣ is a unique consequence of the Munc13-1^{H567K} mutation. To investigate how this Pᵥᵣ increase in Munc13-1^{H567K}/Munc13-1^{H567K} cells affects the synaptic release probability Pᵣ, Pᵣ was estimated by analyzing the use-dependent, progressive block of NMDA-EPSCs in the presence of MK-801, which reflects the release probability across all synapses of a given neuron (Rosenmund, 1993, Hessler, 1993). Munc13-1^{H567K}/Munc13-1^{H567K} and control cells showed a similar initial fast decay of EPSC amplitudes as a function of stimulus number while the slower component of the EPSC decay appeared to be somewhat, but not significantly, smaller in Munc13-1^{H567K}/Munc13-1^{H567K} cells (FIG. 19D).

These data are best compatible with the interpretation that individual synapses in primary hippocampal neurons contain two pools of releasable vesicles with different Pᵥᵣ, as is the case in adrenal chromaffin cells (Voets, 2001) and the calyx of Held synapse (Neher and Sakaba, 2001; Sakaba and Neher, 2001a and 2001b). The size of the high Pᵥᵣ vesicle pool appears to be similar in Munc13-1^{H567K}/Munc13-1^{H567K} and wild type cells. In contrast, the pool size of low Pᵥᵣ vesicles or their efficacy of release induced by hypertonic solutions seem to be selectively reduced in Munc13-11^{H567K}/Munc13-1^{H567K} mutant cells. Assuming that evoked EPSCs at low frequency are mainly generated by release from the high Pᵥᵣ vesicle pool, this model would explain why evoked EPSCs and EPSC decay during MK-801 are largely unaffected in Munc13-1^{H567K}/Munc13-1^{H567K} mutant cells although their responses to hypertonic sucrose solutions are significantly reduced.

Regardless of whether the above model of a selective reduction in the pool size or release efficacy of low Pᵥᵣ vesicles is accurate, the significant Pᵥᵣ and non significant Pᵣ the shift observed in Munc13-1^{H567K}/Munc13-1^{H567K} mutant neurons should have profound consequences for synaptic short term plasticity and transmission during high frequency stimulation. The stability of evoked EPSCs at action potential frequencies of 1-10 Hz was therefore monitored. Compared to wild type responses, evoked EPSC amplitudes of Munc13-1^{H567K}/Munc13-1^{H567K} neurons showed pronounced depression (steady state vs. initial amplitude of the train) at all frequencies tested (FIGS. 20A-20C). This aberrantly strong synaptic depression during high frequency stimulation, which was already detectable with the second stimulus, was even more pronounced when tested the characteristics of Munc13-1^{H567K} after Semliki-Forest-Virus mediated overexpression on the Munc13-1/Munc13-2 double deletion mutant background. Synaptic depression at 10 Hz stimulation frequency in Munc13-1/Munc13-2 double mutant cells overexpressing wild type Munc13-1 was comparable to that seen in wild type cells (compare wild type and Munc13-1 rescue traces in FIGS. 20B and 20D, respectively). In contrast, Munc13-1/Munc13-2 double mutant neurons overexpressing Munc13-1^{H567K} showed a degree of synaptic depression that even exceeded that of Munc13-1^{H567K}/Munc13-1^{H567K} mutant neurons (FIG. 20D), most likely because the contribution of wild type Munc13-2 is lacking in these cells.

Taken together, our data on Munc13-1^{H567K}/Munc13-1^{H567K} neurons obtained with high frequency stimulation paradigms demonstrate that an intact C, domain in Munc13-1 is essential for the maintenance of high levels of transmitter release during high frequency action potential trains. Because the principal function of Munc13-1 is to prime synaptic vesicles to fusion competence (Augustin, 1999b), it is likely that the C₁ domain in Munc13-1 contributes to the activity dependent regulation of the readily releasable synaptic vesicle supply by acting as a ligand dependent positive regulator of Munc13-1 function. It was shown that the refilling rate of the readily releasable synaptic vesicle pool as determined by responses to paired hypertonic sucrose pulses (i.e. in the absence of activity) is normal in Munc13-1^{H567K}/Munc13-1^{H567K} neurons (FIG. 19B). A possible role for the Munc13-1 C₁ domain in activity dependent refilling of readily releasable vesicle pools was directly examined by continuously stimulating neurons at 10 Hz and intermittently depleting the readily releasable vesicle pool with a hypertonic sucrose pulse (4 s; FIG. 21A). Following return to normal osmolarity, the EPSC amplitude returned to a steady-state amplitude within seconds. The time course of this recovery was first analyzed. Both Munc13-1^{H567K}/Munc13-1^{H567K} neurons (τ=0.88±0.09 s, n=36, p=0.015) and Munc13-1/Munc13-2 double deficient neurons overexpressing Munc13-1^{H567K} (τ=0.93±0.10 s, n=14, p=0.015) showed a significantly slower time constant compared to wild type neurons (τ=0.61±0.047 s, n=29; FIG. 21B). The recovery from complete depletion of readily releasable vesicle pools in these experiments is not only dependent on the refilling rate of vesicles into the releasable pool but also on the rate of vesicle depletion during the ongoing high frequency stimulation. Thus, these measurements underestimated the actual reduction of the activity dependent refilling kinetics in Munc13-1^{H567K}/Munc13-1^{H567K} neurons and Munc13-1/Munc13-2 double deficient neurons overexpressing Munc13-1^{H567K}, because both types of mutant neurons show much higher depression/depletion rates than wild type cells (FIGS. 20A-20D). A more direct measure of the activity dependent refilling kinetics in these experiments is the initial slope of recovery following return to normal osmolarity, as it represents the rate by which new vesicles arrive. To allow direct comparison of pool refilling between cells, this rate in pool units/s was expressed by normalizing EPSC responses during recovery to the initial EPSC amplitude at the beginning of the train (defined as 1 pool unit). Both Munc13-1^{H567K}/Munc13-1^{H567K} neurons (0.13±0.01 pool units/s, n=36, p<0.001) and Munc13-1/Munc13-2 double deficient neurons overexpressing Munc13-1^{H567K} (0.059±0.01 pool unit/s, n=14, p<0.001) had a strongly reduced refilling kinetics compared to wildtype (0.29±0.027 pool unit/s, n=29, FIG. 21C). These observations indicate that Munc13-1 indeed participates in the activity dependent refilling of readily releasable vesicle pools, with its C₁ domain acting as an activity sensor.

Analyses of release and pool dynamics revealed a surprising insight into the C₁ domain function. It was found that readily releasable vesicle pools are reduced in Munc13-1^{H567K}/Munc13-1^{H567K} neurons under resting conditions (FIG. 19A). However, activity independent refilling of these pools, as measured in a paired sucrose pulse paradigm, was normal (FIG. 19B). This indicates that the Munc13-1^{H567K}/Munc13-1^{H567K} mutation does not simply cause a retardation of vesicle pool refilling rates in the resting state but rather an apparent loss of a subset of releasable vesicles, with the remaining pool being refilled at normal rates. In addition, we observed that evoked EPSC amplitudes are only slightly reduced in Munc13-1^{H567K}/Munc13-1^{H567K} neurons (FIG. 16A) despite an underlying significant reduction in the size of readily releasable vesicle pools (FIG. 19A). This suggests that the remaining vesicle pool has an intrinsically higher vesicular release probability Pᵥᵣ (FIG. 19A) than the average release probability in neurons with an intact Munc13-1 C₁ domain. Consistent with this conclusion is the increased paired pulse depression and stronger depression rate during trains of action potentials seen in Munc13-1^{H567K}/Munc13-1^{H567K} neurons (FIGS. 20A and 20B; compare ratios of second to first response at 1 s and 100 ms intervals).

Without being bound by theory, the best explanation for this consistent but unexpected data set is that hippocampal primary neurons, like chromaffin cells (Voets, 2001) and the calyx of held synapse (Neher, 2001; Sakaba, 2001a and 2001b), form two functionally distinct pools of vesicles that can be released by hypertonic solutions in wild type neurons. (1) One pool is reluctantly releasable (low Pᵥᵣ) but quickly replenished and contributes only weakly to action potential induced release at low stimulation frequencies. Processes such as ongoing synaptic activity or direct activation of the Munc13-1 C₁ domain facilitate release from this pool (see below). In contrast, the Munc13-1^{H567K}/Munc13-1^{H567K} mutation abolishes release from this reluctantly releasable pool and makes it insensitive to hypertonic solutions. (2) The other pool is readily releasable (high Pᵥᵣ) but slowly replenished, the main contributor to evoked release at low stimulation frequencies, unaffected in Munc13-1^{H567K}/Munc13-1^{H567K} cells, and therefore independent of Munc13-1 C₁ domain function. Essentially these two releasable vesicle pools may represent two parallel modes of Munc13-1 action. Assuming that Munc13-1 promotes core complex formation by unfolding or ‘activating’ Syntaxin (Brose, 2000), both vesicle pools would emerge from a common reserve pool by Munc13-1 mediated priming, i.e. trans core complex assembly. Although the exact molecular determinants of the two releasable pools in hippocampal neurons remains unknown, it is likely that they differ with respect to the presence of additional facilitatory components of the release machinery that may only be present in limited amounts and restricted to the high Pᵥᵣ release sites.

The effects of the Munc13-1^{H567K}/Munc13-1^{H567K} mutation on the two different releasable vesicle pools has striking consequences for the activity dependent regulation of transmitter release in mutant neurons. Munc13-1^{H567K}/Munc13-1^{H567K} mutant neurons showed markedly increased synaptic depression rates and reduced steady state responses when continuously stimulated at or above 1 Hz (FIGS. 20A-20D), and refilling of readily releasable vesicle pools during ongoing high frequency stimulation was slowed down (FIGS. 21A-21C). These findings indicate that Munc13-1, in addition to mediating basal synaptic vesicle priming (Augustin, 1999b), participates in the maintenance of releasable vesicle pools during high action potential frequencies (see Weis, 1999, for a model of Ca⁺⁺-dependent vesicle pool dynamics and short term synaptic depression). This adaptation mechanism is dependent on an intact C₁ domain, suggesting that high frequency stimulation causes a cascade of successive events involving Ca⁺⁺ influx, activation of phospholipase C, and transient increases in synaptic diacylglycerol levels, which in turn boost Munc13-1 priming activity. The fact that Munc13-1^{H567K}/Munc13-1^{H567K} mice die immediately after birth suggests that the C₁ domain dependent stimulation of Munc13-1 activity and the concomitant adaptation to high activity levels is important for neurons involved in essential body functions such as rhythmically active nerve cells in the respiratory system. Like its role under resting conditions, this activity dependent function of the Munc13-1 C₁ domain can be explained by the differential effects of the Munc13-1 C₁ domain on the low and high Pᵥᵣ vesicle pools described above. In fact, the observed increases in paired pulse depression, synaptic depression rate at 1-10 Hz stimulation frequencies, and lower steady state amplitude during trains of stimuli are predicted consequences of the increased apparent Pᵥᵣ seen in Munc13-1^{H667K}/Munc13-1^{H567K} neurons (FIGS. 19A and 19C). The assumption that high action potential frequencies or direct activation by β-PEs lead to the C, domain dependent ‘activation’ of low Pᵥᵣ vesicles for secretion would explain the reduction in activity dependent refilling rates in Munc13-1^{H567K}/Munc13-1^{H567K} neurons (FIGS. 20A-20C). Essentially, the Munc13-1 C₁ domain serves as an activity sensor that responds to increases in diacylglycerol levels or exogenous β-PEs by making an otherwise reluctantly releasable vesicle pool available for evoked secretion and thereby allowing a neuron to adapt to ongoing high activity levels.

Binding of Syntaxin to Munc13-1 is not affected by β-PEs. However, diacylglycerol/β-PE binding by Munc13-1 is known to modulate its interaction with the synaptic vesicle protein DOC2α (Orita, 1997) and β-PE effects on Munc13-1 are partly mediated by the Munc13-1/DOC2α interaction (Hori, 1999). Thus, the ‘activated’ low Pᵥᵣ vesicles that are recruited for evoked secretion during ongoing synaptic activity or following β-PE treatment could be a synaptic vesicle population that, in addition to core complexes in the trans state, is attached to Munc13-1 via DOC2α in a diacylglycerol/β-PE dependent manner. The phenotype of DOC2α deletion mutant mice would be compatible with this view as it is characterized by enhanced synaptic depression at 5 Hz stimulation frequency, indicating an increased apparent release probability (e.g. due to a reduction in a low Pᵥᵣ vesicle pool) (Sakaguchi, 1999).

The present examples show that regulation of Munc13-1 function by diacylglycerol is important for the maintenance of readily releasable vesicle pools during high action potential frequencies. This regulatory mechanism appears to be of crucial importance for one or more neuronal networks involved in the control of essential body functions because Munc13-1^{H567K}/Munc13-1^{H567K} mice die immediately after birth.

The upregulation of Munc13-1 activity by diacylglycerol does also occur in single autaptic glutamatergic neurons. One obvious mechanism underlying this regulatory process would be presynaptic positive autofeedback regulation of phospholipase C via metabotropic glutamate receptors. However, such positive autofeedback has not been demonstrated yet. Alternatively, it is possible that the C₁ domain dependent upregulation of Munc13-1 in autaptic neurons is due to the increased Ca²⁺ concentrations that accumulate during the high frequency stimulation and directly activate phospholipase C.

### EXAMPLE 20

### Functional Calmodulin-Binding Sites in Unc13-Members

Members of the UNC-13/Munc13 family of synaptic vesicle priming proteins are regulated by the second messenger DAG or DAG-analogues via the C₁ domain, that is present in all known family members. The C₁ domain of UNC-13/Munc13 proteins therefore defines a site that can be targeted by molecules to modify the efficacy of the secretion process.

To identify additional sites in members of this protein family that can be targeted to modify UNC/Munc13 function, a region within these proteins that was reported to bind Calmodulin in drosophila UNC13 (Xu et al.) was analyzed.

Calmodulin is an abundant protein in all eukaryotic cells, which regulates the function of its targets in response to changes in the intracellular calcium concentration. Given the here identified and described calcium- and activity-dependent regulation of UNC13 family proteins on secretion, the possibility that modulation of secretion may also occur via Calmodulin mediated interaction of UNC13/Munc13 was examined. So far the precise site and type of interaction between UNC13s and calmodulin are not characterized, nor was an evolutionarily conservation of interaction shown. Furthermore, no information about the function of this interaction is available.

Calmodulin is an ubiquitous intracellular signaling molecule, which interacts with a large number of proteins in calcium-dependent or -independent manners. While some of the calmodulin binding sites on target proteins can be identified by computer-assisted profile search, this method was unsuccessful for the prediction of the binding site at Drosophila DUNC-13 (REF) and Munc13-1/ubMunc13-2. An alternative screening method was developed to identify the exact calmodulin binding site based on surface distribution of hydrophobic and hydrophilic residues along alpha-helical regions of the N-terminus of Munc13-1 and ub-Munc13-2. Using this search an evolutionarily conserved Calmodulin binding motif, which is present in some but not all members of the protein family was identified. Munc13-1, ubMunc13-2, DUNC-13 and C. elegans UNC-13 contain this motif (see FIG. 22). Based on the identification of residues predicted to be essential for Calmodulin binding to UNC13 homologues, we identified a point mutation in Munc13-1 and ubMunc13-2 that should abolish Calmodulin binding. This mutation was used in biochemical essays to confirm the loss of calmodulin binding activity (FIG. 23, 24). Finally, to test whether this calmodulin binding has functional consequences, virus mediated expression of mutated Munc13-1 and ubMunc13-2 proteins in neurons that lack endogenous Munc13 proteins were used (FIG. 25). By comparison of the rescue characteristics of wildtype and mutant proteins we assessed the function of the Calmodulin binding site. The results show that both the identified DAG/phorbol ester binding C1 region, as well as the calmodulin binding site are essential for activity-dependent short-term plasticity. However, only the C1 binding site is essential for phorbol ester/DAG induced enhancements of synaptic responses. With this additional regulatory site in the N-terminus present in some UNC13 members, further development of isoform specific- and function dependent regulation of secretory events by pharmacological means are possible. It will be possible to regulate specifically the activity dependence of synaptic transmission (by interfering with the calmodulin binding site) or the overall gain of a synapse (by interaction with the C1 domain).

Calmodulin binding motifs can be defined by the presence of hydrophobic residues that are interspersed in a certain pattern into amphipathic helices (Rhoads et al.). An alignment of the UNC/Munc13 proteins of several species indicates that a Calmodulin recognition motif may be present in the N-terminus of a number of isoforms of the protein family. Those residues that are characteristic for a Calmodulin binding motif are conserved whereas other residues indispensable for the putative calmodulin interaction are not (see FIG. 22).

To determine whether the putative calmodulin binding motif is functional, peptides based on the putative binding site were synthesized and their properties in biochemical binding experiments were examined. The peptides used were synthesized and purified according to standard procedures. The following peptides were used:

| | | |
|---|---|---|
| Munc13-1: | crakanwlrafnkvrmqlqear | (SEQ ID NO: 11) |
| ubMunc13-2: | cqarahwfravtkvrlqlqeis | (SEQ ID NO: 12) |

The tryptophane fluorescence of the peptides was used as a reporter for Calmodulin binding in an experimental setup that was essentially identical to the experiments described elsewhere. (O'Neil et al.). Excitation of 10 μM solutions of the peptides in 0.25 M NH₄Acetate pH 8 and 1 mM EGTA at 290 nm (bandwidth 1 nm) produced a maximum in the emission (bandwidth 8 nm) of both peptides at 355 nm. Upon addition of equimolar amounts of Apocalmodulin (Calcium-free Calmodulin) or Ca²⁺/Calmodulin the emission spectra of both peptides were again determined (FIG. 23). Addition of Apocalmodulin to the peptide reflecting the Calmodulin binding site of Munc13-1 leads to a pronounced blue shift and slight intensity increase of the emission spectrum, indicating that Calmodulin binds to the peptide and makes contact to the tryptophane residue, thereby changing its spectral properties. Upon addition of excess calcium, the intensity of the 325 nm maximum was further increased, indicating tighter binding of Calmodulin to the Munc13-1 derived peptide in the presence of calcium. The same experiment was performed with the peptide derived of ubMunc13-2. This peptide also bound Ca²⁺/Calmodulin as shown in FIG. 23, however, binding for this peptide was absolutely dependent on the presence of calcium. Together these results indicate that Munc13-1 and ubMunc13-2 contain a functional Calmodulin binding motif_. In view of the evolutionarily conservation of the motif, other members of the protein family are likely to be Calmodulin targets, too. This data (FIG. 23) show that Calmodulin interacts with some Unc13 derivatives also in a Calcium-independent manner (such as Munc13-1) as well as in a strict Calcium-dependent manner (ubMunc13-2).

### EXAMPLE 21

### Analysis of the Function of the Calmodulin Binding Site

To analyze the function of the Calmodulin binding site in members of the UNC13/Munc13 family the Calmodulin binding properties of specific Munc13 proteins was modified in a way that does not interfere with other Calmodulin target interactions. Such a tool is a point mutation in a Munc13 protein that abolishes Calmodulin binding. On basis of the evolutionarily conservation of the tryptophane residues (FIG. 22), and since these hydrophobic residues make contact to Calmodulin, we created expression constructs that contain W464R mutation in case of Munc13-1 and a W387R mutation in case of ubMunc13-2. The effect of these mutations on Calmodulin binding was determined in cosedimentation experiments (FIG. 24). GST-tagged fragments of Munc13-1 (aa 445-567) or ubMunc13-2 (aa 372494) were expressed in bacteria and purified according to standard protocols. 15 μg of each expressed fusion protein were used to test for Calmodulin binding from 2 ml of a rat brain synaptosome extract. Rat brain synaptosomes were extracted with Igepal (1%) at a protein concentration of 2 mg/ml in a buffer containing Tris 50 mM pH 8, NaCl 150 mM, EGTA 1 mM and a cocktail of protease inhibitors. The cosedimentation experiment was performed as described prebviously (Betz, Neuron 30 (2002) 183-196).

Calmodulin from brain extract robustly bound to GSTMunc13-1 (aa 445-567) and GSTubMunc13-2 (aa 372-494) in the presence of 3 mM Ca (FIG. 24) This binding is not attributable to the GST tag since GST alone does not bind Calmodulin. GST-fusion proteins that contain the tryptophane mutations express equally well but show no Calmodulin binding within the sensitivity of this assay. Hence Calmodulin binding is virtually abolished by mutation of evolutionarily conserved tryptophanes within the Calmodulin binding motif of members of the UNC/Munc13 family. Specific mutations analyzed herein comprised a TRYPTOPHAN (W, TRP) to Arginine (R, ARG)-mutation (substitution) in position W464 of SEQ ID NO: 9 (Munc13-1 of rat), W387 of SEQ ID NO: 10 (Munc13-2 of rat), W376 of SEQ ID NO: 11 (Munc13-2 of human), W498 of SEQ ID NO: 12 (DUNC13 of Drosophila) and W593 of SEQ ID NO: 13 (Unc13 of C. elegans).

Functional consequences of Calmodulin binding deficient Unc13s.

The activity-dependent maintenance of synaptic transmission as well as the phorbol ester responsiveness of Calmodulin binding mutants was tested. The Munc13-1/Munc13-2 double deficient mutant mice were used to obtain neuronal cultures void of any endogenous Munc13 priming factor. Synaptic transmission was reconstituted/rescued by semliki forest virus mediated overexpression of Munc13 isoforms and their mutant derivatives. For this purpose, the ubMunc13-2 isoform was used for rescue experiments, because of its more robust regulation of synaptic transmission compared to the Munc13-1 isoform. First, the modulation of synaptic amplitudes during 10 Hz action potential trains was examined. UbMunc13-2 driven synapses show robust augmentation of synaptic responses. However, as also shown for the C1-DAG binding deficient mutants, mutation at of the Calmodulin binding site (W387R) converted the augmentation into strong depression of synaptic responses (FIG. 25). This indicates that Unc13 function is strongly modulated by both the interaction with Calmodulin as well as diacylglycerol. Surprisingly, we found that the degree of phorbol ester induced potentiation, a measure of the regulation of synaptic responses by the C1-domain, was not majorly affected by the calmodulin binding mutant. Compared to the phorbol ester induced potentiation of wildtype ubMunc13-2 (PDBU at 1 μM for 1 minute: 3.9±1.1 fold, n=20), the phorbol ester response was very robust in the Calmodulin binding deficient mutant ubMunc13-2(W387R) (2.9±0.4 fold, n=9) This is in contrast to the C1-DAG binding mutant ubMunc13-2(H491K) that had no detectable potentiation of evoked responses by phorbol esters (1.04±0.03; n=21). These data suggest that the maintenance of synaptic responses during trains of action potentials requires both the activation of the Calmodulin binding site as well as interaction of the C1 domain with DAG in the plasma membrane. However, the requirement of modulating synaptic responses can be directly influenced by ligand acting at the C1 domain, therefore bypassing the requirement of Calmodulin interaction at the N-terminus. This finding is furthermore supported by measurements of synaptic properties from Munc13-1/Munc13-2 double deficient neurons that overexpress ubMunc13-2 deletion constructs that lack the first 400 amino acids. Neurons rescued with this construct also showed 43±3% (n=18) depression of synaptic responses during 10 Hz action potential trains, while the phobol ester response was intact (4.5±1.3 fold increase; n=18).

Accordingly, a second interaction site on Unc13-isoforms (FIG. 24) was identified, that can be used to differentially modulate both the activity dependence (via the calmodulin site) as well as the overall gain of synaptic transmission (via the C1 site).

### REFERENCES

Ashery, 2000, EMBO J., 19, 3586-96
Asztely, 2000, Eur. J. Neurosci., 12, 662-9
Augustin, 1999, Biochem. J., 337, 363-71
Augustin, 2001, J Neurosci, 21, 10-7.
Augustin, 1999, Nature, 400, 457-61
Bennett, 2000, Prog Neurobiol, 60, 109-37.
Betz, 1998, Neuron, 21, 123-36
Betz, 2001, Neuron, 30, 183-96.
Brose, 1995, J. Biol. Chem., 270, 25273-80
Clements, 2000, Trends Neurosci., 23, 105-13
Cochilla, 1999, Annu Rev Neurosci, 22, 1-10
Craig, 2001, Nat Neurosci, 4, 569-78.
Davis, 1994, Trends Neurosci., 17, 9-13
Delaney, 1994, J. Neurosci., 14, 5885-902
Fabbri, 1994, J Biol Chem, 269, 26848-57.
Fisher, 1997, Trends Neurosci., 20, 170-7
Gillis, 1996, Neuron, 16, 1209-20
Goda, 1996, Learn Mem, 3, 182-7.
Gorg, 1988, Electrophoresis, 9, 531-46.
Harris, 1989, J. Neurosci., 9, 2982-97
Hawkins, 1993, Annu Rev Neurosci, 16, 625-65
Heinemann, 1993, Pflügers Arch., 424, 105-12
Helmchen, 1996, Biophys. J., 70, 1069-81
Hens, 1995, J Neurochem, 64, 1127-36.
Hessler, 1993, Nature, 366, 569-72
Hoffman, 1998, J Neurosci, 18, 3521-8.
Honda, 2000, J Physiol, 529 Pt 3, 763-76.
Hori, 1999, J Neurosci, 19, 7262-7.
Huang, 1997, J. Neurophysiol., 78, 2870-80
Iwasaki, 2000, J Biochem (Tokyo), 128, 407-14.
Kawakami, 2000, Biochem Biophys Res Commun, 279, 293-7.
Kazanietz, 2000, Biochem Pharmacol, 60, 1417-24.
Kreitzer, 2000, J. Neurosci., 20, 1348-57
Krucker, 2000, Proc. Natl. Acad. Sci. USA, 97, 6856-61
Lackner, 1999, Neuron, 24, 335-46.
Laemmli, 1970, Nature, 227, 680-5.
Lakso, 1996, Proc Natl Acad Sci USA, 93, 5860-5.
Ma, 1999, Nat. Neurosci., 2, 24-30
Magleby, 1976, J. Physiol. (London), 257, 449-70
Majewski, 1998, Prog Neurobiol, 55, 463-75.
Malenka, 1999, Science, 285, 1870-4.
Malgaroli, 1999, Nat. Neurosci., 2, 3-5
Miller, 1999, Neuron, 24, 323-33.
Minami, 1998, J Physiol, 512, 219-25.
Murthy, 1997, Neuron, 18, 599-612
Nayak, 1999, Adv Neurol, 79, 645-58
Neher, 2001, J. Neurosci., 21, 444-61
Nurrish, 1999, Neuron, 24, 231-42.
Nussinovitch, 1988, J. Physiol. (London), 396, 435-55
Oleskevich, 2000, J. Physiol. (London), 524, 513-23
Oleskevich, 2000, J Physiol, 526 Pt 2, 349-57.
Orita, 1997, J Biol Chem, 272, 16081-4.
Parfitt, 1993, J Physiol, 471, 245-68.
Pouzat, 1997, J. Neurosci., 17, 9104-12
Redman, 1997, J Physiol, 501, 41-8.
Reyes, 1998, Nat. Neurosci., 1, 279-85
Reyes, 1999, J. Neurosci., 19, 3827-35
Rosenmund, 1993, Science, 262, 754-7
Rosenmund, 1996, Neuron, 16, 1197-207
Ryan, 1999, J. Neurosci., 19, 1317-23
Sakaba, 2001, Proc Natl Acad Sci USA, 98, 331-6
Sakaguchi, 1999, Eur J Neurosci, 11, 4262-8.
Scanziani, 1998, Proc. Natl. Acad. Sci. USA, 95, 12004-9
Schikorski, 1997, J. Neurosci., 17, 5858-67
Shimazaki, 1996, J Biol Chem, 271, 14548-53.
Smith, 1998, Neuron, 20, 1243-53
Soderling, 2000, Trends Neurosci, 23, 75-80.
Song, 1998, Kidney Int, 53, 1689-95.
Stevens, 1998, Neuron, 21, 885-93
Stevens, 1995, Proc. Natl. Acad. Sci. USA, 92, 846-9
Stevens, 1999, Neuron, 22, 139-46
Sudhof, 1995, Nature, 375, 645-53
Thomson, 1997, J. Physiol. (London), 502, 131-47
Towbin, 1992, Biotechnology, 24, 145-9
Varela, 1997, J. Neurosci., 17, 7926-40
Voets, 2001, Neuron, 28, 537-45
von Rüden, 1993, Science, 262, 1061-5
Walmsley, 1998, Trends Neurosci., 21, 81-8
Wang, 1998, Nature, 394, 384-8
Waters, 2000, J. Neurosci., 20, 7863-70
Weis, 1999, Biophys J, 77, 2418-29.
Xu, 1998, J Biol Chem, 273, 31297-307.
Yawo, 1999, J Physiol, 515, 169-80.
Yawo, 1999, J Physiol, 516, 461-70.
Zucker, 1973, J. Physiol. (London), 229, 787-810
Zucker, 1989, Annu. Rev. Neurosci., 12, 13-31
Zucker, 1996, Neuron, 17, 1049-55
Zucker, 1999, Curr. Opin. Neurobiol., 9, 305-13

1. A method for identifying and/or obtaining a molecule which is capable of modifying secretion processes comprising the steps of
a) contacting an unc-13 molecule or (a) part(s) or (a) fragment(s) thereof with a candidate molecule;
b) measuring and/or detecting a response; and
c) comparing said response to a standard response as measured in the absence of said candidate molecule.

2. A method for identifying and/or obtaining a isoform-specific modulator of Munc-13-1 activity comprising the steps of:
a) contacting a Munc-13-1 molecule or (a) part(s) or (a) fragment(s) thereof with a candidate molecule;
b) measuring and/or detecting a response; and
c) comparing said response to a standard response as measured in the absence of said candidate molecule;
d) contacting the identified and/or obtained candidate molecule of step c) with Munc13-2 or (a) part(s) or (a) fragment(s) thereof or Munc13-3 or (a) part(s) or (a) fragment(s) thereof;
e) measuring and/or detecting whether said candidate molecule interacts with said Munc13-2 or (a) part(s) or (a) fragment(s) thereof or said Munc13-3 or (a) part(s) or (a) fragment(s) thereof; and
f) selecting a candidate molecule which is not capable of interacting with Munc13-2 or (a) part(s) or (a) fragment(s) thereof or with Munc13-3 or (a) part(s) or (a) fragment(s).

3. A method for identifying and/or obtaining a isoform-specific modulator of Munc-13-2 activity comprising the steps of:
a) contacting a Munc-13-2 molecule or (a) part(s) or (a) fragment(s) thereof with a candidate molecule;
b) measuring and/or detecting a response; and
c) comparing said response to a standard response as measured in the absence of said candidate molecule;
d) contacting the identified and/or obtained candidate molecule of step c) with Munc13-1 or (a) part(s) or (a) fragment(s) or Munc13-3 or (a) part(s) or (a) fragment(s) thereof;
e) measuring and/or detecting whether said candidate molecule interacts with said Munc13-1 or (a) part(s) or (a) fragment(s) thereof or said Munc13-3 or (a) part(s) or (a) fragment(s) thereof; and
f) selecting a candidate molecule which is not capable of interacting with Munc13-1 or (a) part(s) or (a) fragment(s) thereof or said Munc13-3 or (a) part(s) or (a) fragment(s).

4. A method for identifying and/or obtaining a isoform-specific modulator of Munc-13-3 activity comprising the steps of:
a) contacting a Munc-13-3 molecule or (a) part(s) or (a) fragment(s) thereof with a candidate molecule;
b) measuring and/or detecting a response; and
c) comparing said response to a standard response as measured in the absence of said candidate molecule;
d) contacting the identified and/or obtained candidate molecule of step c) with Munc13-1 or (a) part(s) or (a) fragment(s) thereof or Munc13-2 or (a) part(s) or (a) fragment(s) thereof;
e) measuring and/or detecting whether said candidate molecule interacts with said Munc13-1 or (a) part(s) or (a) fragment(s) thereof or said Munc13-2 or (a) part(s) or (a) fragment(s) thereof; and
f) selecting a candidate molecule which is not capable of interacting with Munc13-1 or (a) part(s) or (a) fragment(s) thereof or with Munc13-2 or (a) part(s) or (a) fragment(s).

5. The method of any one of claims 1 to 4, further comprising the steps of
a) contacting the identified and/or obtained candidate molecule with (a) protein kinase(s) C or (a) part(s) or (a) fragment(s) thereof;
b) measuring and/or detecting whether said candidate molecule interacts with said (a) protein kinase(s) C or (a) part(s) or (a) fragment(s) thereof, and
c) selecting a candidate molecule which is not capable of interacting with (a) protein kinase(s) C or (a) part(s) or (a) fragment(s) thereof.

6. The method of any one of claims 1 to 5, further comprising the steps of
a) contacting the identified and/or obtained candidate molecule with a calmodulin-binding site;
b) measuring and/or detecting whether said candidate molecule interacts with said calmodulin-binding site; and
c) selecting the candidate molecule which is capable of interacting with said calmodulin-binding site.

7. The method of claim 1, whereby said unc-13 molecule is selected from group consisting of Xenopus Unc-13, Drosphila dUnc-13, C. elegans Unc13, mouse Munc-13, rat or human Munc13.

8. The method of claim 7, whereby said mouse, rat or human Munc-13 is Munc13-1, Munc-13-2 or Munc13-3.

9. The method of any one of claims 4 to 8, whereby said Munc13-1 is encoded by a nucleic acid molecule encoding a polypeptide as shown in SEQ ID NO: 1, 2 or 3.

10. The method of any one of claims 4 to 8, whereby said Munc13-2 is encoded by a nucleic acid molecule encoding a polypeptide as shown in SEQ ID NO.: 4, 5, 6 or 7.

11. The method of any one of claims 4 to 8, whereby said Munc13-3 is encoded by a nucleic acid molecule encoding a polypeptide as shown in SEQ ID NO.: 8.

12. The method of claim 1 or 7, wherein said part or fragment of unc-13 comprises the C1-region of unc13.

13. The method of anyone of claim 2 to 11, wherein said part or fragment of Munc13-1, Munc13-2 or Munc13-3 comprises the C1-region of Munc13-1, Munc13-2 or Munc13-3.

14. The method of anyone of claims 1 to 13, wherein said molecule which is capable of modifying secretion processes or said isoform-specific modulator is an antagonist or an agonist.

15. A composition comprising
a) Munc13 polypeptide or a fragment thereof or a polynucleotide encoding a Munc13 polypeptide or a fragment thereof, or
b) comprising an antibody specifically detecting a Munc 13 polypeptide or
c) comprising an antagonist or an agonist as identified and/or obtained by the method of claim 14.

16. The composition of claim 15 which is a pharmaceutical composition.

17. The composition of claim 15 which is a diagnostic composition.

18. Use of an antagonist or an agonist of unc13 or of a Munc13-isoform as identified and/or obtained by the method of claim 14 for the preparation of a pharmaceutical composition for the treatment of a neurological or a secretarial disorder or disease.

19. Use of a Munc13 polypeptide or a fragment thereof or a polynucleotide encoding a Munc13 polypeptide or a fragment thereof for the preparation of a pharmaceutical composition for the treatment of a neurological or a secretarial disorder or disease.

20. Use of a polynucleotide encoding a Munc-13 polypeptide or a fragment thereof for the preparation of a diagnostic composition for detecting a neurological or a secretarial disorder or disease.

21. Use of an antibody or a fragment or a derivative thereof, an receptor or an aptamer specifically detecting or binding to a Munc-13 molecule for the preparation of a diagnostic composition for detecting a neurological or a secretarial disorder or disease.

22. Use of an antibody or a fragment or a derivative thereof, an receptor or an aptamer specifically detecting or binding to a Munc-13 molecule for the preparation of a pharmaceutical composition for treating, preventing or ameliorating neurological or a secretarial disorder or disease.

23. A method for diagnosing a disese or disorder associated with a modified secretion process comprising the steps of
a) measuring the expression level or activity of Unc13 or a Munc13-isoform in a tissue or cell sample; and
b) correlating the measured expression level or activity with a reference sample representing a tissue- or cell sample not affected by said disease or disorder or with a reference sample representing a tissue- or cell sample affected by said disease or disorder.

24. The method of claim 23, wherein said disease or disorder associated with a modified secretion process is a neurological disease or disorder or is a secretarial disease or disorder.

25. The use of any one of claims 18 to 24, wherein said neurological disease or disorder is selected form the group consisting of schizophrenia, epilepsy, Parkinson's disease, Alzheimer's disease, Huntington's disease, and (ischemic) stroke.

26. The use of any one of claims 18 to 25, wherein said secretorial disease or disorder is selected from the group consisting of diabetes mellitus, Morbus Addison, hypothryodism, hyperthryodism, Morbus Cushing, hypertonus and diabetic nephropathy

27. A method for the preparation of a pharmaceutical composition comprising the steps of the method of anyone of claims 1 to 14 and, additionally, formulating the identified and/or obtained molecule in a pharmaceutically acceptable form.

28. A method for preventing, ameliorating and/or treating a neurological or a secretarial disorder or disease comprising the administration of Munc13 polypeptide or a fragment thereof or of a polynucleotide encoding a Munc13 polypeptide or a fragment thereof or comprising the administration of a molecule as identified by the method of any one of claims 1 to 14.

29. Use of a transgenic, non-human animal for identifying and/or obtaining a molecule which is capable of modifying secretion processes and/or which is an isoform-specific modulator.

30. The use of claim 29, wherein said transgenic, non-human animal comprises in its somatic and/or germ cells at least one gene encoding Unc13 (or a part or a fragment thereof), Munc13-1 (or a part or a fragment thereof), Munc13-2 (or a part or a fragment thereof) or Munc13-3 (or a part or a fragment thereof).

31. The use of claim 29, wherein said transgenic, non-human animal does not express a functional Unc13 (or a part or a fragment thereof), a functional Munc13-1 (or a part or a fragment thereof), a functional Munc13-2 (or a part or a fragment thereof) or a functional Munc13-3 (or a part or a fragment thereof).

32. The use of claim 30, wherein said transgenic, non-human animal is a Munc13-1 “knock-in” animal.

33. The use of claim 31, wherein said transgenic, non-human animal is a Munc13-2 “knock-out” animal.


SEQUENCE LISTING (12 sequences)

SEQ ID NO: 1 - protein, 282 aa, Mus musculus
MSLLCVGEKFNTYVTLKVQNVKSTTIAVRGSQPSWEQDFMFEINRLDLGLTVEVWNKGLIWDKMVGTVWIPLRTIRQSNEEGPGEWLTLDSQAIMADSEICGTKDPTFHRILLDAHFELPLDIPEEEARYWAKKLEQLNAMRDQDEVSHLQYSFQDQQDKPLPVPSSQCCNWNYFGWGEQNDDPDSAVDDRDSDYRSDTSNSIPPPYYTIRSPTPLCTSTPCDRPLWGPGSPTVTPCTAMRSSLSRGRSGGCGAGECAGMHGLISQNISIYILKLHLFCLGA

SEQ ID NO: 2 - protein, 282 aa, Homo sapiens; identical to SEQ ID NO: 1

SEQ ID NO: 3 - protein, 1735 aa, Rattusnorvegicus
MSLLCVGVKKAKFDGAQEKFNTYVTLKVQNVKSTTIAVRGSQPSWEQDFMFEINRLDLGLTVEVWNKGLIWDTMVGTVWIPLRTIRQSNEEGPGEWLTLDSQAIMADSEICGTKDPTFHRILLDAHFELPLDIPEEEARYWAKKLEQLNAMRDQDEYSFQDQQDKPLPVPSSQCCNWNYFGWGEQNDDPDSAVDDRDSDYRSETSNSIPPPYYTTSQPNASVHQYSVRPPPLGSRESYSDSMHSYEEFSEPRALSPTGSSRYASSGELSQGSSQLSEDFDPDEHSLQGSELDDERDRDSYHSCHSSVSYHKDSPRWDQDEEDLEDLEDLEDEELPEEEELEEEELEEEEELEEEELELEEEEEVPDDLASYTQQEDTTVAEPKEFKRISFPTAAPQKEDKVSAVPIEAPDVSKGIPKAATPEEKAAAECAQEAEPPKSEESFRSREAEEGQEGQDAMSRAKANWLRAFNKVRMQLQEARGEGEMSKSLWFKGGPGGGLIIIDSMPDIRKRKPIPLVSDLAMSLVQSRKAGITSALASSTLNNEELKNHVYKKTLQALIYPISCTTPHNFEVWTATTPTYCYECEGLLWGIARQGMRCTECGVKCHEKCQDLLNADCLQRAAEKSSKHGAEDRTQNIIMVLKDRMKIRERNKPEIFELIQEVFAVTKSAHTQQMKAVKQSVLDGTSKWSAKISITVVCAQGLQAKDKTGSSDPYVTVQVGKTKKRTKTIYGNLNPVWEENFHFECHNSSDRIKVRVLDEDDDIKSRVKQRFKRESDDFLGQTIIEVRTLSGEMDVWYNLDKRTDKSAVSGAIRLHISVEIKGEEKVAPYHVQYTCLHENLFHFVTDVQNNGVVKIPDAKGDDAWKVYYDETAQEIVDEFAMRYGVESIYQAMTHFACLSSKYMCPGVPAVMSTLLANINAYYAHTTASTNVSASDRFAASNFGKERFVKLLDQLHNSLRIDLSMYRNNFPASSPERLQDLKSTVDLLTSITFFRMKVQELQSPPRASQVVKDCVKACLNSTYEYIFNNCHELYGREYQTDPAKKGEVPPEEQGPSIKNLDFWSKLITLIVSIIEEDKNSYTPCLNQFPQELNVGKISAEVMWSLFAQDMKYAMEEHDKHRLCKSADYMNLHFKVKWLYNEYVAELPTFKDRVPEYPAWFEPFVIQWLDENEEVSRDFLHGALERDKKDGFQQTSEHALFSCSVVDVFSQLNQSFEIIKKLECPDPQIVGHYMRRFAKTISNVLLQYADIVSKDFASYCSKEKEKVPCILMNNTQQLRVQLEKMFEAMGGKELDAEASGTLKELQVKLNNVLDELSHVFATSFQPHIEECVRQMGDILSQVKGTGNVPASACSSVAQDADNVLQPIMDLLDSNLTLFAKICEKTVLKRVLKELWKLVMNTMERTIVLPPLTDQTMIGTLLRKHGKGLEKGRVKLPSHSDGTQMIFNAAKELGQLSKLKDHMVREEAKSLTPKQCAVVELALDTIKQYFHAGGVGLKKTFLEKSPDLQSLRYALSLYTQATDLLIKTFVQTQSAQVHGGKGTRFTLSEDVCPEMGSGVEDPVGEVSVHVELFTHPGTGEQKVTVKVVAANDLKWQTSGIFRPFIEVNIVGPQLSDKKRKFATKSKNNSWAPKYNESFQFSLSADAGPECYELQVCVKDYCFAREDRTVELAVLQLRELAQRGSAACWLPLGRRIHMDDTGLTVLRILSQRSNDEVAKEFVKLKSDTRSAEEGGAAPAP

SEQ ID NO: 4 - protein, 1591 aa, Mus musculus
MSLLCVRVKRAKFQGSPDKFNTYVTLKVQNEKSTTVAVRGDQPSWEQDFMFEISRLDLGLSVEVWNKGLIWDTMVGTVWIALKTIRQSDEEGPGEWSTLEAETLMKDDEICGTKNPTPHKILLDTRFELPFDIPEEEARYWTYKLEQINALADDNEYSSQEESQRKPLPTAAAQCSFEDPDSAVDDRDSDYRSETDNSAPPPYHTTTQPNASVHQFPVPVRWPQQLFLQGSSHDSCNDSMQSYDLDYPEHRALSPTSSSRYGSSCNVSQGSSLLSELDQYHEQDDDGRERDSIHSSHSYGSLSKDGQAGLGEQEKALEVTCESEKEKTGESKEMRDDATIHPPSDLVLHKDHVLGPQESLPEETASSPFTQARAHWFRAVTKVRLQLQEISDDGDPSLPQWLPEGPAGGLYGIDSMPDLRRKKPLPLVSDLSLVQSRKAGITSAMATRTSLKDEELKSHVYKKTLQALIYPISCTTPHNFEVWSATTPTYCYECEGLLWAVARQGMRCSECGVKCHEKCQDLLNADCLQRAAEKSSKHGAEDRTQNIIMAMKDRMKIRERNKPEIFRIIRDVFTVSKVAHVQQMKTVKQSVLDGTSKWSAKITITVVCAQGLQAKDKTGSSDPYVTVQVGKTKKRTKTIFGNLNPVWEEKFHFECHNSSDRIKVRVWDEDDDIKSRVKQRLKRESDDFLGQTIIEVRTLSGEMDVWYNLEKRTDKSAVSGAIRLQISVEIKGEEKVAPYHVQYTCLHENLFHYLTDIQGSGGVWIPEARGDDAWKVYFDETAQEIVDEFAMAYGIESIYQAMTHFACLSSKYMCPGVPAVMSTLMANINAYYAHTTASTNVSASDRFAASNFGKERFVKLLDQLHNSLRIDLSTYRNNFPAGSPERLQDLKSTVDLLTSITFFRMKVQELQSPPRASQVVKDCVKACLNSTYEYIFNNCHDLYSHQYQLQEQPLEEPGPSIRNLDFWPKLITLIVSIIEEDKNSYTPVLSQFPQELNVGKVSAEVMWHLFAQDMKYALEEHEKDRLCKSADYMNLHFKVKWLHNEYVRDLPALQGQVPEYPGAWFEQFVLQWLDENEDVSLEFLRGALERDKKDGFQQTSEHALFSCSVVDVFTQLNQSFEIIRKLECPDPNILAHYMRRFAKTIGKVLMQYADILSKNFPAYCTKERLPCILMNNMQQLRVQLEKMFEAMGGKELDSEAADSLKELQVKLNTVLDELSMVFGNSFQVRIDECVRQMADILGQVRGTGNASPNARASVAQDADSVLRGVMDFLDGNLTLFATVCEKTVLKRVLKELWRVVMNTMERVIVLPPLTDQTGTQLILTAAKELSQLSKLKDHMVREETRNLTPKQCAVLDLALDTIKQYFHAGGNGLKKTFLEKSPDLQSLRYALSLYTQTTDTLIKTFVGSQTAQGAGVDDPVGEVSIQVDLFTHPGTGEHKVTVKVVAANDLKWQTAGMFRPFVEVTMVGPHQSDKKRKFTTRSKSNNWTPKYNETFHFLLGNEEGPEAYELQICVKDYCFAREDRVIGLAVMPLRDVAAKGSCACWCPLGRKIHMDETGMTILRILSQRSNDEVAREFVKLKSESRSTEEGS

SEQ ID NO: 5 - protein, 1591 aa, Homo sapiens [sequence omitted]

SEQ ID NO: 6 - protein, 1622 aa, Rattus norvegicus
MSLLCVRVKRAKFQGSPDKFNTYVTLKVQNVKSTTVAVRGDQPSWEQDFMFEISRLDLGLSVEVWNKGLIWDTMVGTVWIALKTIRQSDEEGPGEWSTLEAETLMKNDEICGTKNPTPHKILLGTRFELPFDIPEEEARYWTYKLEQINALADDNEYSSQEESQRKLLPTAAAQCRHWTYLGWGEHQTFEDPDSAVDDRDSDYRSETSNSAPPPYHTTTQPNASAHQFPMPVPLPQQLFLQGSSRDSCNDSMQSYDLDYPERRALSPTSSSRYGSSCNVSRGSSLLSELDQYHEQDDDGRERDSIHSSHSYGSLSRDGQAGLGEQEKALEVACESQKEKTGESKERDDATVCPPSDLMLHKDLTLGPQESFPEEKASSPFTQARAHWFRAVTKVRLQLQEISDDGDPSLPQWLPEGPAGGLYGIDSMPDLRRKKPLPLVSDLAMSLVQSRKAGITSAMATRTSLKDEDLKSHVYKKTLQALIYPISCTTPHNFEVWSATTPTYCYECEGLLWGLARQGMRCSECGVKCHEKCQDLLNADCLQRAAEKSSKHGAEDRTQNIIMAMKDRMKIRERNKPEIFEVIRDVFTVSKVAHVQQMKTVKQSVLDGTSKWSAKITITVVCAQGLQAKDKTGSSDPYVTVQVGKTKKRTKTIFGNLNPVWEEKFHFECHNSSDRIKVRVWDEDDDIKSRVKQRLKRESDDFLGQTIIEVRTLSGEMDVWYNLEKRTDKSAVSGAIRLQINVEIKGEEKVAPYHVQYTCLHENLFHYLTDIQGSGGVWIPDARGDDAWKVYFDETAQEIVDEFAMRYGIESIYQAMTHFACLSSKYMCPGVPAVMSTLLANINAYYAHTTASTNVSASDRFAASNFGKERFVKLLDQLHNSLRIDLSTYRNNFPAGSPERLQDLKSTVDLLTSITFFRMKVQELQSPPRASQVVKDCVKACLNSTYEYIFNNCHDLYSHQYQLQEQPLEEPGPSIRNLDFWPKLITLIVSIIEEDKNSYTPVLNQFPQELNVGKVSAEVMWHLFAQDMKYALEEHEKDRLCKSADYMNLHFKVKWLHNEYVRELPALQGQVPEYPAWFEQFVLQWLDENEDVSLEFLRGGLERDKRDGFQQTSEHALFSCSVVDVFTQLNQSFEIIRKLECPDPSILAHYMRRFAKTIGKVLIQYADILSKNFPAYCTKERLPCILMNNMQQLRVQLEKMFEAMGGKELDSEAADSLKELQVKLNTVLDELSMVFGNSFQVRIDECVRQMADILGQVRGTGNASPNARASVAQDADSVLRPLMDFLDGNLTLFATVCEKTVLKRVLKELWRVVMNTMERVIVLPPLIDQTGTQLILTAAKELSQLSKLKDHMVREETRNLTPKQCAVLDLALDTVKQYFHAGGNGLKKTFLEKSPDLQSLRYALSLYTQTTDTLIKTFVRSQTAQVHDGKGIRFTANEDIRPEKGAGVDDPVGEVSIQVDLFTHPGTGEHKVTVKVVAANDLKWQTAGMFRPFVEVTMVGPHQSDKKRKFTTKSKSNSWTPKYNETFHFLLGNEEGPEAYELQICVKDYCFAREDRVIGLAVMPLRDVAAKGSCACWCPLGRKIHMDETGMTILRILSQRSNDEVAREFVKLKSESRSIEEGS

SEQ ID NO: 7 - protein, 1985 aa, Rattus norvegicus
MKRLLRESEEEIMLTLGPSSSLSPDQVRTETVCIVKGKSTGPTGSLPEDNFPPPCCESADSTTSGERDRNLAQLGSFEQQASSQPSLACTACASGSDSRELSPASITSCSEPSERNKARPIFPRGPGQRCRHEHQEPLGDVVEYIIRELQGISRLQSEIAELQQHLNQVRGSVDEVSSCVDSVLSEIEGLHVGSSSLGKVRHGEKAQELHVERSREEAILYLYGLPEHDGESTVELVDNFLAKHLCVNGMQCNRYVREAYRAGTAPAPRPTVVKLVHPEHRDLILQKSILLQSVGVRVATREEPVWPEGCKNPPKESLSCLQQFQDHSRNHQGKPALQLETGNRRQMSGPHQMRTQNQHRELQASEHQGLSFLPKDGSAKQSDVSKLQDEVKGTSGAPQVISDPCGELSLLHQLEGSSPVLIPKEEDCGKLQIFKQDSQEHKACNVTKLQSDCNNAIKASSCLSLSGPLKAEKVNAEDRMLGGEDGLDILSPKQLEDLLADKSRRFATLNPDSAVEEVIIGPETFSNMVHIDLNEEETCTAQVLKNVFDKSSCVLGGSQEDEDVEIKFHTTKLSRAIHHFRLALQGVFQKLENNGSISPEDLESNESGSQSENSDRLLWTVSSGGAHDCSVESPASQGSESLLSVVSGGVGISVQGDQTPQAPSNFSLASNNSPLTNSLLSFPLAPGLGNETCSRPDSPNQGKLSLEQVCAETIYLNKCINNFKNVLREKRLRQKKLLQELVQTASHLSVEDIPSEGKREALQISDDGDPSLPQWLPEGPAGGLYGIDSMPDLRRKKPLPLVSDLAMSLVQSRKAGITSAMATRTSLKDEDLKSHVYKKTLQALIYPISCTTPHNFEVWSATTPTYCYECEGLLWGLARQGMRCSECGVKCHEKCQDLLNADCLQRAAEKSSKHGAEDRTQNIIMAMKDRMKIRERNKPEIFEVIRDVFTVSKVAHVQQMKTVKQSVLDGTSKWSAKITITVVCAQGLQAKDKTGSSDPYVTVQVGKTKKRTKTIFGNLNPVWEEKFHFECHNSSDRIKVRVWDEDDDIKSRVKQRLKRESDDFLGQTIIEVRTLSGEMDVWYNLEKRTDKSAVSGAIRLQINVEIKGEEKVAPYHVQYTCLHENLFHYLTDIQGSGGVWIPDARGDDAWKVYFDETAQEIVDEFAMRYGIESIYQAMTHFACLSSKYMCPGVPAVMSTLLANINAYYAHTTASTNVSASDRFAASNFGKERFVKLLDQLHNSLRIDLSTYRNNFPAGSPERLQDLKSTVDLLTSITFFRMKVQELQSPPRASQVVKDCVKACLNSTYEYIFNNCHDLYSHQYQLQEQPLEEPGPSIRNLDFWPKLITLIVSIIEEDKNSYTPVLNQFPQELNVGKVSAEVMWHLFAQDMKYALEEHEKDRLCKSADYMNLHFKVKWLHNEYVRELPALQGQVPEYPAWFEQFVLQWLDENEDVSLEFLRGGLERDKRDGFQQTSEHALFSCSVVDVFTQLNQSFEIIRKLECPDPSILAHYMRRFAKTIGKVLIQYADILSKNFPAYCTKERLPCILMNNMQQLRVQLEKMFEAMGGKELDSEAADSLKELQVKLNTVLDELSMVFGNSFQVRIDECVRQMADILGQVRGTGNASPNARASVAQDADSVLRPLMDFLDGNLTLFATVCEKTVLKRVLKELWRVVMNTMERVIVLPPLIDQTGTQLILTAAKELSQLSKLKDHMVREETRNLTPKQCAVLDLALDTVKQYFHAGGNGLKKTFLEKSPDLQSLRYALSLYTQTTDTLIKTFVRSQTAQVHDGKGIRFTANEDIRPEKGAGVDDPVGEVSIQVDLFTHPGTGEHKVTVKVVAANDLKWQTAGMFRPFVEVTMVGPHQSDKKRKFTTKSKSNSWTPKYNETFHFLLGNEEGPEAYELQICVKDYCFAREDRVIGLAVMPLRDVAAKGSCACWCPLGRKIHMDETGMTILRILSQRSNDEVAREFVKLKSESRSIEEGS

SEQ ID NO: 8 - protein, 2204 aa, Rattus norvegicus
MVASLFKSLILAYIHKLCKGMFTKKLGNTTKKKENRQQNKDQDFPTAGHTKPPKLSNALKSTVKKIAKCSSTRNFSVEDEEGHKDFSLSPTFSYRVAIANGLQTAVTNSDEDLLQELSSIESSYSESFNELRSSTENQVQSTHTMPVRRNRKSSSSLAPSEGSSDGERTLHTLKLGALRKLRKWKKSQECVSSDSELSTVKKTWGIRSKSLDRTARNPKTNVLEPGFSSSGCISQTHDVMEMIFKELQGISQIETELSELRGHVNALKYSIDEISSSVEVVQSEIEQLRTGFVQARRETRDIHDYIKHLGHMGSKVSLRFLNVPEERHEYVESVVYQILVDKMGFSDVPNAIKIEFAQRIGQQRDCPNAKPRPILVYFETPQQRDSVLKKSYKLKGTGIAISTDILTYDIRERKEKGVLPSSQTYESMDMKLSTPEPKAKKNAWLSPNDSDRELESDLSRSSYADSPAKGSSSKSSSKSHSARSKNKAANSRTSQKSDYNKRPSKPPASEKPTPHYVEATPLWHSQSDFFTPKLSRSESDFSKLCQSYSEDFSESQFFCRTNGSSLLSSSDRELWQRKQEGMTALYHSPQDQGLDGNIPTVPGQGEIENTETVDSGMSNSVVCASGDRSNYSGSQLSLHEDLSPWKEWNQAGHGTDGLDSSTQEPFDYDTNSLSDQQLDMSSKDLDDLGKCHSDLQDDSESYDLTQDDNSSPCPGLDNEPQGQWVGQYDSYQKTNSNDLYPNQSHPSMMYRSQSELQSDDSEAAQPKSWHSRLSIDLSDKTFKFPKFGSTLQRAKSALEVVWNKSTQSLSGCEDSGSSLMGRFRTLSQSTANESSTTLDSDIYTEPYYYKAEEEEDYCEPVADSETDYVEVMEQVLAKLENRTSVTEVDEHIKEYDHPSYETPYETPQDEGYDGQADDIISEGELETLNEPAVEMELVEDESQNLPVEPPEVMKPKRIRPSFKEAALRAYKKQMAELEEKILAGDSSSVDEKARIVSGNDLDASKFSALQVFGGAGRGLYGIDSMPDLRRKKTLPIVRDVAMTLAARKSGLSLAMVIRTSLNNEELKMHVFRKTLQALIYPISSTTPHNFEVWTATTPTYCYECEGLLWGIARQGMKCLECGVKCHEKCQDLLNADCLQRAAEKSSKHGAEDKTQTIITAMKERMKIRERNRPEVFEVIQEMFQISKEDFVQYTKAAKQSVLDGTSKWSAKITITVVSAQGLQAKDKTGSSDPYVTVQVGKNKRRTKTIFGNLNPVWDEKFYFECHNSTDRIKVRVWDEDDDIKSRVKQHFKKESDDFLGQTIVEVRTLSGEMDVWYNLEKRTDKSAVSGAIRLKINVEIKGEEKVAPYHIQYTCLHENLFHYLTEVKSNGSVKIPEVKGDEAWKVFFDDASQEIVDEFAMRYGVESIYQAMTHFSCLSSKYMCPGVPAVMSALLANINAFYAHTTVSTNVQVSASDRFAATNFGREKFIKLLDQLHNSLRIDLSKYRENFPASNSERLQDLKSTVDLLTSITFFRMKVLELQSPPKASAVVKDCVRACLDSTYKYIFDNCHELYSQLIDPSKKQDVPREEQGPTTKNLDFWPQLITLMVTIIDEDKTAYTPVLNQFPQELNMGKISAEIMWSLFALDMKYALEEHEKQRLCKSTDYMNLHFKVKWFYNEYVRELPAFKDAVPEYSLWFEPFVMQWLDENEDVSMEFLHGALGRDKKDGFQQTSDHALFSCSVVDVFAQLNQSFEIIKKLECPNPEALSHLMRRFAKTINKVLVQYAAIVSSDFSSYCDKETVPCILMNNIQQLRVQLEKMFESMGGKELDPEASTILKELQIKLNGVLDALSVTYGESFQLVIEECIKQMGAELNQMRANGNSAANKNNAAMDAEIVLRPLMDFLDKILSLSAKICEKTVLKRVLKELWKLVLNKIEKQIVLPPLTDQTGPQMIFIAAKELGQLSKLKEHMIRDDAKGLTPRQCAIMEVVLATIKQYFHAGGNGLKKNFLEKSPDLHSLRYALSLYTQTTDALIKKFIETQGSQSRSSKDAVGQISVHVDVTTTPGTGEHKVTVKVIAINDLNWQTTTMFRPFVEVCMLGPSLGDKKRKQGTKTKSNTWSPKYNETFQFILGNENRPGAYELHLSVKDYCFAREDRIIGMTVIQLQNIAEKGSYGAWYPLLKNLSMDETGLTILRILSQRTSDDVAKEFVRLKSETRSIDESA

SEQ ID NO: 9 - protein, 1752 aa, Drosophila melanogaster
MILTNEMVNIGMLDSESEFGLIENSYSYQVPDNEQIDSVNSYNNKTQNVTNNGIEKANTKNKPVPLHDPPTKKASTVGMFGSILGKAAAAVQSATQAVNQSASSVASVVAQKPTIVPRTNNVLLLASVCSPNEIKRNSSSVEFDSEYGYQMPDVESLSSHYANTGGDYDNSNMKIHEFGTYARYRPYADYHTNGNQSQFKEEAVIPGEPEVINTNILPIGPQATGKKLPTVNGKSALLIKQMPTEVYDDESDTDELDVSPSTGKVPSYSIYSVQEDYYMDLQQTTPSIQPNGFYEQVNNGYDYREDYFNEEDEYKYLEQQREQEEHNQPKNKKYLKQAKISKIQPPSLDFIDVGQDDDFIYDNYHSEDDSGNYLEGSSSGSVGPIEGSIIKVDSNIEASFASLNKKSDSFTPTNDSLQKHDTVIGESTTKLTRLRTEKMCPDVDEEDENLSDHVSDLTDLSKLISQKKKTLLRGETEEVVGGHMQVLRQTEITARQRWHWAYNKIIMQLNNGGGPGEVGLRTNGHPGDNPFYSNIDSMPDIRPRRKSIPLVSELTMAATKRNAGLTSCLFHVQRLNDEDLKMHVYKKALQALIYPISSTTPHNFLLWTATSPTYCYECEGLLWGIARQGVRCTECGVKCHEKCKDLLNADCLQRAAEKSSKHGAEDKANSIITAMKDRMKQREREKPEIFELIRMTFGVDPDTHIDSLEQAEHAPVEGTSKWSCKLTITVICAQGLIAKDKSGTSDPYVTVQVSKVKKRTRTMPQELNPVWNEKFHFECHNSSDRIKVRVWDEDNDLKSKLRQKLTRESDDFLGQTIIEVRTLSGEMDVWYNLEKRTDKSAVSGAIRLHISVEIKGEEKVAPYHVQYTCLHENLFHYLCEENTGMVKLPTQKGDDAWKLYFDEIPEEIVDEYSMRYGIENNYQAMTHFHCLSAKYMCPGVPAVMSTLLANINAYYAHTTASSAVSASDRFAASNFGKEKFVKLLDQLHNSLRIDLSMYRNNFPASSPEKLMDLKSTVDLLTSITFFRMKVQELSSPPRASTVVKDCVKACLRSTYQFLFENCYELYNREFQVDPNEAKRAPDDHEPKLDSADFWHKLIALIVSVIDEDKNSYGTGLNQFPQELNIGQLSASSMGHLFAVDMKYALREEHEAHRLCKSAAYMNLHFRVKWLYSNYVKEVPPYKGAVPDYPAWFEPFVMQWLNENDDVSLEYLHGAFKRDKKDGFQKSSEHALFSNSVVDVFTQLTQCFDVVSKLECPDPEIWKRYMRRFAKTIVKVLIAYADIGKLEFPEHMKDERIACILMNNIQQLRVQLEKMFESMGGGELEEDAAHILKELQQNLTFALDDLTSQFRYQFGAPNNFIQCENFGNMLLFIKRRQWNPCCGNLAAQRNAVAVEADEVLRPLMDLLDGSLTLYAQSCEKTVLKRLLKELWKIVMRILEKTIVLPPMTDKTMMFKHLTDNAKNLASNAKIEDMGRLFKSHMAGKQDVKSALSGVMDISKEVEKNLSPKQCAVLDVALDTIKQYFHAGGNGLKKTFLEKSPELQMLRYALSLYTQMTDTLIKTFISSQVHEVDLENSEESVGEISVQIDLFSHPGTGEHKVNVKVVAANDLKWQIPSGMFRPFVDINLIGPHLQEKKRKFATKSKSNNWSPKYNESFSFTIGNEEQLDFFELHICVKDYCFARDDRLVGVAVIPLKDISEKGSVACWLPLMRRIEMDETGWTILRILSQRNNDEVAKEFVKLKSEIRQEPTMGT

SEQ ID NO: 10 - protein, 1813 aa, C. elegans
MDDVGDYNDDQLSDGMSMRLLCITIKKARLQGAVDEFNSYVTVKLQTVKSTTVAVRGNLPCWEQEFIFETNRPDDGMVLELWAKGVLWDKLIGVHYMPLSEIRYSNAAGSGQWLQMDHELETRNGQTVGTRGPTGHNLLTDVRFELPFDVQGHDEDIQSRLLALNGLIEHDQLGPNNHHRAPFNHSGLSEDSDYTSDVSVPVNHHQLHPNSSAHQYESHLHPHRTRQLLHTREGAASYEDEEDAYHARHQPESDDYNHQDTYDQHSSYYNDEYAPSGSSSQYQRQGYQDQDQQHQNIYEDTVTPVREFGESAVPPAASSSRRQFDQVYGYASSSEERYDTPMSSGRLPRDEPILEHSEPEYVYDQNGYPEEDNYGINPTYSEDHFEGQTNDYSTTHQEPNDFRNDYNSSYQREYWNESEPLSYNSRPPNGHIRTGANTWREPSTSSRPTSSQAWNYQDDTHQYDEVDRGSRVSFTRTPSVDRTDRPSESGGGFYDEMSESGRPGRPDSHHNWRYDSIQEEDNEKDNWKQHVEGYEEGQEEKQKDNQKPNDHSAASPQDHYHRSDSTAQQDFGNNIVRQTIQEEEEKRNYQELWHNAYKRVCADLGIKSTVLDGNGSSAANAFYKSIDAAPNMNVARTKTSIPLVSELTMATKRAQAGLANAARTTFSDTELKTHVYKKTLQALIYPISATTPHNFATTTFQTPTFCYECEGLLWGLARQGLRCTQCQVKVHDKCRELLSADCLQRAAEKSTKHGEADRTQSLVNVIRDRMKIQEQNKPEVFQMIRTVFDVDENIQKETLKTVKASILEGSSKWSAKITLTVLCAQGLIAKDKTGKSDPYVTAQVGKTKRRTRTIHQELNPVWNEKFHFECHNSTDRIKVRVWDEDNDLKSKLRQKLTRESDDFLGQTVIEVRTLSGEMDVWYNLEKRTDKSAVSGAIRLHINVEIKGEEKLAPYHVQYTCLHEHLFAAHCVDEEVKLPKVRGEDSWKVCFQETGQEIAEEFAMRYGIESIYQAMTHFACLCTRYMCAGVPAVLSTLLANINAYYAHTTATSAVSAPDRFAASNFGKERFVKLLDQLHNSLRIDLSAYRNHFPSSSPAKLQDLKSTVDLLTSITFFRMKVLELASPPRASTVVRECAKACMQQTYQLMFESCAEQFPILDTSVQFWYEFIDYIMRVIEEDQKNYTPALNQFPQELNVGNLSAETLWSMYKNDLKMALEEHAQKKRCKTPEYMNLYFKVKGFYFKYVADLSTYKSSIPEFPAWFIPFVMDWLNENDEHSMDILRNAYNVDKADNFPQTSEHTKFSNSVVDVFTQLNAALKLLKQMDCPNPEVAADMMKRFSKTLNKVLLAYADMVQKDFPKFAHDEKLACILMNNVQQLRVQLEKIYETMGGAELDEHIGQVLTVLQKKLNSVLDRLSAEFVTTLEPHIHEQTIKLGMLLVKIKGPQLQKTQVQPEADAVLEPLMDLLEGSLRRYADQCEKTVLKYILKELWKITIVNMEKRVVLPPLSDKALLKQLPNAKIGDVTKLMSTNIQSIKGMNSVKDMMDMARESEKSLTPRQCTVLDCALDAIKDSFHASGKGLKKSFFEKSPELQSLKYALSLYTQTTEQLIKTFITSQRQQDLPSQEQPVGEVSVQVDLFSHPGTGEQKVTVKILAANDLRWQTSSAFKPFVEVHLVGPHLSDKKRKWSTKTKAGNWAPKFNETFHFFLGNEGEPEHYELMFQVKDYCFARDDRVVGVGVLQLSSVVDQAGSCAMWVQLGTRLHIDETGLILLRILSQRQTDEVAKDFVRLKTECRYETETVMAASASSQNINRT

SEQ ID NO: 11 - protein, 22 aa, Artificial sequence
  Description of Artificial Sequence Munc13-1 peptide
CRAKANWLRAFNKVRMQLQEAR

SEQ ID NO: 12 - protein, 22 aa, Artificial sequence
  Description of Artificial Sequence ubMunc13-2 peptide
CQARAHWFRAVTKVRLQLQEIS